(19) United States
(12) Patent Application Publication
(10) Pub. No.: US 2026/0335361 A1
(43) Pub. Date: Oct. 29, 2026

(54) Methods and Treatment of Traumatic Brain Injury Accompanied by Hemorrhagic Shock

(71) Applicant: Hemanext Inc., Lexington, MA (US)

(72) Inventors: Andrew DUNHAM, Tower Lakes, IL (US); Laurel OMERT, Lexington, MA (US); Tatsuro YOSHIDA, West Newton, MA (US)

(73) Assignee: Hemanext Inc., Lexington, MA (US)

(21) Appl. No.: 19/113,421

(22) PCT Filed: Sep. 20, 2023
(86) PCT No.: PCT/US2023/074641

§ 371 Date: Mar. 19, 2025

Related U.S. Application Data
(60) Provisional application No. 63/408,460, filed on Sep. 20, 2022.
(60) Provisional application No. 63/583,607, filed on Sep. 19, 2023.

(51) Int. Cl. A61K 35/14; A61P 7/04; A61P 9/00; A61P 25/00

(52) U.S. Cl. CPC A61K 35/14; A61P 7/04; A61P 9/00; A61P 25/00

(57) ABSTRACT

The present disclosure relates to methods for treating or stabilizing a traumatic brain injury (TBI) accompanied by hemorrhagic shock in a patient in need thereof by administration of oxygen reduced blood to the patient.

### CROSS-REFERENCE TO RELATED APPLICATIONS

This application claims the benefit of U.S. Provisional Application No. 63/408,460, filed Sep. 20, 2022, and U.S. Provisional Application No. 63/583,607, filed Sep. 19, 2023, both of which are incorporated by reference in their entireties herein.

### FIELD OF THE INVENTION

The present disclosure relates to treatment of traumatic brain injury accompanied by hemorrhagic shock.

### BACKGROUND OF THE INVENTION

Death from hemorrhage and hemorrhagic shock represents a substantial health care problem globally, with more than 60,000 deaths per year in the United States and an estimated 1.9 million deaths per year worldwide, the vast majority of which involve hemorrhagic shock resulting from physical trauma. See Lozano et al., “Global and regional mortality from 235 causes of death for 20 age groups in 1990 and 2010: a systematic analysis for the Global Burden of Disease,” Lancet, 15; 380(9859):2095-128 (2012). The causes of hemorrhagic shock vary widely and include trauma, maternal hemorrhage, gastrointestinal hemorrhage, perioperative hemorrhage (e.g., surgery-related hemorrhage), and rupture of an aneurysm. See Mannucci et al., “Prevention and treatment of major blood loss,” New England Journal of Medicine, 356:2301-11 (2007) (“Mannucci”); Hunt et al., “Bleeding and coagulopathies in critical care,” New England Journal of Medicine, 370:847-859 (2014) (“Hunt”); Halmin et al., “Epidemiology of massive transfusion: a binational study from Sweden and Denmark,” Critical Care Medicine, 44:468-477 (2016) (“Halmin”); and Ruseckaite et al., “Descriptive characteristics and in-hospital mortality of critically bleeding patients requiring massive transfusion: results from the Australian and New Zealand Massive Transfusion Registry,” Vox sanguinis 112:240-248 (2017). If hemorrhagic shock is not reversed in time, severe blood loss can result in inadequate oxygen delivery at the cellular level and may quickly lead to death.

Of the different types of trauma that can lead to hemorrhagic shock, traumatic brain injury (TBI) is one of the most significant in terms of its associated rates of morbidity and mortality in the United States. According to the Centers for Disease Control and Prevention, more than 3 million traumatic brain injuries occur annually in the U.S., resulting in about 2.5 million emergency department visits, more than 280,000 hospitalizations, and over 52,000 deaths. See Centers for Disease Control and Prevention, “Surveillance Report of Traumatic Brain Injury-related Emergency Department Visits, Hospitalizations, and Deaths,” Surveillance Report (2014). Overall, TBI has an economic impact of around 76.5 billion dollars per year in direct and indirect medical expenses. See Taylor et al., “Traumatic brain injury—related emergency department visits, hospitalizations, and deaths—United States,” MMWR Surveillance Summaries, 6(9):1-16 (2017).

Despite advances in trauma resuscitation over the past several decades, TBI and hemorrhagic shock remain leading causes of death in civilian and military populations. See Mannucci; Hunt; and Halmin. The combination of TBI and hemorrhagic shock is associated with worsened coagulopathy prior to resuscitation and increased rates of morbidity and mortality compared to hemorrhagic shock alone. See Galvagno et al., “Outcomes after concomitant traumatic brain injury and hemorrhagic shock: A secondary analysis from the Pragmatic, Randomized Optimal Platelets and Plasma Ratios Trial,” Journal of Trauma and Acute Care Surgery, 83(4):668-674 (2017). Moreover, the optimal resuscitation strategy for a traumatic brain injury accompanied by hemorrhagic shock is unclear. While immediate blood transfusion generally constitutes the best course of action to treat hemorrhaging trauma patients by restoring perfusion to ischemic tissues and preventing hypoxic and ischemic damage induced by the hemorrhagic shock, treatment of TBI accompanied by hemorrhagic shock is complicated by the fact that blood transfusion may worsen TBI-related brain pathology, leading to cerebral edema and diffuse swelling in the brain, as well as loss of local and systemic autoregulatory mechanisms that can limit the recovery of hemodynamics after resuscitation. See Falk, “Fluid resuscitation in brain-injured patients,” Critical Care Medicine, 23(1):4-6 (1995). Cerebral edema is one of the most prominent pathophysiological factors associated with death and unfavorable outcomes after a TBI. See Feldmann et al., “The prognostic value of intracranial pressure monitoring after severe head injuries,” Acta neurochirurgica Supplementum, 28(1):74-7 (1979); and Saul et al., “Effect of intracranial pressure monitoring and aggressive treatment on mortality in severe head injury,” Journal of Neurosurgery, 56(4):498-503 (1982). Other adverse outcomes of TBI include damage to the central nervous system (CNS) through various mechanisms including synaptic dysfunction, protein aggregation, mitochondrial dysfunction, oxidative stress, neuroinflammation, and reduced oxygen cerebral metabolic rate due to ischemia, inadequate oxygen delivery, and/or depressed metabolism. Oxygen deprivation in the brain can quickly lead to neuronal death. Transfusion of fresh red blood cells (RBCs) to a subject suffering from a TBI has been shown to support microcirculatory perfusion and to improve tissue oxygenation, mitochondrial function, and cerebral blood flow. See Bramlett et al., “Long-Term Consequences of Traumatic Brain Injury: Current Status of Potential Mechanisms of Injury and Neurological Outcomes,” Journal of Neurotrauma, 32(23):1834-48 (2015); and Hakiminia et al., “Oxidative stress and mitochondrial dysfunction following traumatic brain injury: From mechanistic view to targeted therapeutic opportunities,” Fundamental & Clinical Pharmacology, 36(4):612-662 (2022). For at least the reasons discussed above, the use of high quality blood to treat traumatic brain injuries accompanied by hemorrhagic shock is of paramount importance.

However, the supplies of liquid blood and blood components are currently limited by storage systems used in conventional blood storage practices. When stored conventionally, stored blood undergoes a steady deterioration which is associated with various storage lesions including, among others, hemolysis, hemoglobin degradation, and reduced ATP and 2,3-DPG concentrations. When transfused into a patient, the effects of the steady deterioration during storage manifest, for example, as a reduction in the 24-hour in vivo recovery. The rapid decrease in the hematocrit that results from reduced 24-hour recovery, when severe, can result in delayed hemolytic transfusion reaction (DHTR). Other complications, for example systemic inflammatory response syndrome (SIRS), transfusion related acute lung injury (TRALI), and transfusion related immunomodulation (TRIM) are associated with transfusion of stored blood, though identification of the underlying causes has remained unclear.

Even when transfused within the current 6-week limit, stored blood tends to exhibit lower quality (e.g. increased fraction of red blood cells removed; compromised oxygen exchange capacity; reduced deformability) and increased toxicity, often manifested as the clinical sequelae of transfusion therapy. A large number of articles in the literature supports this view. See e.g., Zimring, “Established and theoretical factors to consider in assessing the red cell storage lesion,” Blood, 125:2185-2190 (2015); Zhu et al., “Impaired adenosine-5′-triphosphate release from red blood cells promotes their adhesion to endothelial cells: a mechanism of hypoxemia after transfusion,” Critical care medicine, 39:2478-2486 (2011); Weinberg et al., “Red blood cell age and potentiation of transfusion-related pathology in trauma patients,” Transfusion, 51:867-873 (2011); Spinella et al., “Does the storage duration of blood products affect outcomes in critically ill patients?” Transfusion 51:1644-1650 (2011); Roback et al., “Insufficient nitric oxide bioavailability: a hypothesis to explain adverse effects of red blood cell transfusion,” Transfusion, 51:859-866 (2011); Reynolds et al., “The transfusion problem: role of aberrant S-nitrosylation,” Transfusion, 51:852-858 (2011); Kim-Shapiro et al., “Storage lesion: role of red blood cell breakdown,” Transfusion, 51:844-851 (2011); Jy et al., “Microparticles in stored red blood cells as potential mediators of transfusion complications,” Transfusion, 51:886-893 (2011); Hod et al., “Transfusion of human volunteers with older, stored red blood cells produces extravascular hemolysis and circulating non-transferrin-bound iron,” Blood, 118:6675-6682 (2011); Flegel et al., “Does prolonged storage of red blood cells cause harm?” British journal of haematology 165:3-16 (2014); Redlin et al., “Red blood cell storage duration is associated with various clinical outcomes in pediatric cardiac surgery,” Transfusion medicine and hemotherapy: offizielles Organ der Deutschen Gesellschaft fur Transfusionsmedizin und Immunhamatologie 41:146-151 (2014); Rogers et al., “Storage duration of red blood cell transfusion and Clostridium difficile infection: a within person comparison,” PLOS One 9:e89332 (2014); Spinella et al., “Properties of stored red blood cells: understanding immune and vascular reactivity,” Transfusion 51:894-900 (2011); Brown et al., “Length of red cell unit storage and risk for delirium after cardiac surgery,” Anesth Analg, 119:242-250 (2014); Wang et al., “Transfusion of older stored blood worsens outcomes in canines depending on the presence and severity of pneumonia,” Transfusion, 54:1712-1724 (2014); Liu et al., “Mechanism of faster NO scavenging by older stored red blood cells,” Redox biology, 2:211-9 (2014); Prestia et al., “Transfusion of stored blood impairs host defenses against Gram-negative pathogens in mice,” Transfusion 54:2842-2851 (2014); D'Alessandro et al., “An update on red blood cell storage lesions, as gleaned through biochemistry and omics technologies,” Transfusion, 55:205-219 (2015) (hereby incorporated by reference in their entireties). An extensive body of in vitro studies unequivocally shows the degradation of red blood cells (storage lesions) during conventional storage. A body of emerging metabolomic studies show the development of storage lesions at the molecular level. See Roback et al., “Metabolomics of AS-1 RBCs Storage,” Transfusion medicine reviews (2014); D'Alessandro et al., “Metabolomics of AS-5 RBCs supernatants following routine storage,” Vox sanguinis (2014); D'Alessandro et al., “Routine storage of red blood cell (RBC) units in additive solution-3: a comprehensive investigation of the RBC metabolome,” Transfusion 55:1155-1168 (2015); D'Alessandro et al., “Red blood cell storage in additive solution-7 preserves energy and redox metabolism: a metabolomics approach,” Transfusion (2015); Wither et al., “Hemoglobin oxidation at functional amino acid residues during routine storage of red blood cells,” Transfusion (2015); D'Alessandro et al., “Citrate metabolism in red blood cells stored in additive solution-3,” Transfusion (2016); D'Alessandro et al., “Omics markers of the red cell storage lesion and metabolic linkage,” Blood Transfus, 15:137-144 (2017); Yoshida et al., “Red blood cell storage lesion: causes and potential clinical consequences,” Blood Transfusion, 17(1):27-52 (2019); and Reilly et al., “Potential Consequences of the Red Blood Cell Storage Lesion on Cardiac Electrophysiology,” Journal of the American Heart Association, 9(21):e017748 (2020) (hereby incorporated by reference in their entireties). There is a need for reducing or preventing this degradation to increase the efficacy of transfusions (more O₂ delivery to peripheral tissues immediately after transfusion) and to reduce mortality due to TBI accompanied by hemorrhagic shock.

Oxidative damage initiates many red blood cell (RBC) storage lesions in conventionally stored blood and their downstream consequences; thus, methods to reduce the extent of oxidative stress are required to reduce the RBC storage lesions. A number of approaches have been developed aimed at minimizing storage lesions and improving transfusion outcomes. Approaches include additive solutions (for example, U.S. Pat. No. 4,769,318, to Hamasaki et al.; U.S. Pat. No. 4,880,786, to Sasakawa et al.; and U.S. Pat. No. 6,447,987, to Hess et al.) and frozen storage (for example, U.S. Pat. No. 6,413,713, to Serebrennikov; Chaplin et al., “Blood Cells for Transfusion,” Blood, 59:1118-20 (1982); and Valeri et al., “The survival, function, and hemolysis of human RBCs stored at 4 degrees C in additive solution (AS-1, AS-3, or AS-5) for 42 days and then biochemically modified, frozen, thawed, washed, and stored at 4 degrees C in sodium chloride and glucose solution for 24 hours,” Transfusion, 40:1341-5 (2000)) (hereby incorporated by reference in their entireties).

One approach that has proven successful in improving blood quality and extending its utility is through the depletion of oxygen and storage under anaerobic conditions. Among the benefits of storing blood under oxygen depleted conditions are improved levels of ATP and 2,3-DPG, and reduced hemolysis. See U.S. Pat. No. 5,624,794, to Bitensky et al.; U.S. Pat. No. 6,162,396, to Bitensky et al.; and U.S. Pat. No. 5,476,764, to Bitensky (hereby incorporated by reference in their entireties). U.S. Pat. No. 5,789,151, to Bitensky et al. is directed to blood storage additive solutions (hereby incorporated by reference in its entirety). U.S. Pat. No. 6,162,396 (the '396 patent) discloses anaerobic storage bags for blood storage that comprise an oxygen impermeable outer layer, a red blood cell (RBC) compatible inner layer that is permeable to oxygen, and having an oxygen scrubber placed between the inner and outer layers.

Storing blood under oxygen depleted conditions can also result in reduced microparticle levels, reductions in the loss of deformability, reduced lipid and protein oxidation, and higher post transfusion survival when compared to blood stored under conventional conditions. See Yoshida et al., “The effects of additive solution pH and metabolic rejuvenation on anaerobic storage of red cells,” Transfusion 48:2096-2105 (2008); and Yoshida, T., et al. “Reduction of microparticle generation during anaerobic storage of red blood cells,” Transfusion, 52:83A (2012) (hereby incorporated by reference in their entireties). Anaerobically stored RBCs further provide higher 24-hour in vivo recovery after autologous transfusion, higher 2,3-DPG and ATP levels, lower hemolysis, and beneficial remodeling of metabolic pathway. See Reisz et al. “Oxidative modifications of glyceraldehyde 3-phosphate dehydrogenase regulate metabolic reprogramming of stored red blood cells,” Blood, 128:e32-e42 (2016); and Yoshida et al., “Extended storage of red blood cells under anaerobic conditions,” Vox sanguinis 92:22-31 (2007) (hereby incorporated by reference in their entireties).

In the present disclosure, we demonstrate that oxygen reduced blood provides for surprising improvements in clinical outcomes when transfused to treat traumatic brain injury accompanied by hemorrhagic shock. For example, using a rat model of resuscitation after traumatic brain injury accompanied by hemorrhagic shock, we show that oxygen reduced blood provides for reduced organ damage compared to non-oxygen reduced conventionally stored blood. In addition, oxygen reduced blood, when transfused to treat TBI accompanied by hemorrhagic shock, more effectively stabilizes hemodynamics compared to non-oxygen reduced conventionally stored blood.

Oxygen reduced blood provides for improved methods for treatment of traumatic brain injury accompanied by hemorrhagic shock to reverse hemorrhagic shock, thereby reducing mortality and morbidity compared to non-oxygen reduced conventionally stored blood. The improved quality of oxygen reduced blood provides for reduced organ failure, including reductions in levels of markers of lung damage, liver damage, kidney damage, and systemic inflammation compared to non-oxygen reduced conventionally stored blood.

### SUMMARY

The present specification addresses the need to develop effective treatments for traumatic brain injury and traumatic brain injury accompanied by hemorrhagic shock. In an aspect, the present specification provides, and includes, methods of reversing hemorrhagic shock in patients suffering from a traumatic brain injury through transfusion of oxygen-reduced blood.

The present disclosure provides for, and includes, a method of treating a traumatic brain injury (TBI) accompanied by hemorrhagic shock in a patient in need thereof comprising administering oxygen reduced blood having an oxygen saturation (SO₂) of 20% or less prior to and during storage to the patient, where the patient exhibits a Glasgow Coma Scale (GCS) score of 12 or less and has a shock index (SI) of greater than 0.9 prior to the administering, and wherein the patient exhibits a GCS score of at least 13 after the administering.

The present disclosure provides for, and includes, a method of improving hepatic function in a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein the patient has a bilirubin level that is reduced by at least 20% after the administering, compared to a TBI patient undergoing hemorrhagic shock having been administered the same amount of non-oxygen reduced conventionally stored blood.

The present disclosure provides for, and includes, a method of improving splenic function in a traumatic brain injury patient in need thereof, comprising administering oxygen reduced blood having an oxygen saturation (SO₂) of 20% or less prior to and during storage to a patient having a traumatic brain injury accompanied by hemorrhagic shock, wherein a monocyte chemoattractant protein-1 (MCP1) level in the patient is reduced by at least 20% after the administering, compared to a TBI patient undergoing hemorrhagic shock having been administered the same amount of non-oxygen reduced conventionally stored blood.

The present disclosure provides for, and includes, a method of improving cardiac function in a traumatic brain injury patient in need thereof, comprising administering oxygen reduced blood having an oxygen saturation (SO₂) of 20% or less prior to and during storage to a patient having a traumatic brain injury accompanied by hemorrhagic shock, wherein a troponin level in the patient is reduced by at least 5% after the administering compared to a TBI patient undergoing hemorrhagic shock having been administered the same amount of non-oxygen reduced conventionally stored blood.

The present disclosure provides for, and includes, a method of improving pulmonary function in a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein a myeloperoxidase (MPO) level in the patient is reduced after the administering compared to a TBI patient undergoing hemorrhagic shock having been administered the same amount of non-oxygen reduced conventionally stored blood.

The present disclosure provides for, and includes, a method of improving renal function in a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, and wherein a urine creatinine level in the patient is reduced by at least 5% after the administering compared to a TBI patient undergoing hemorrhagic shock having been administered the same amount of non-oxygen reduced conventionally stored blood.

The present disclosure provides for, and includes, a method of increasing a plasma serum cytokine level in a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein the plasma serum cytokine is selected from the group consisting of interleukin 6 (IL-6), CXC motif chemokine ligand 1 (CXCL1), interleukin 10 (IL-10), and any combination thereof.

The present disclosure provides for, and includes, a method of modulating a stress level marker in the plasma of a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein the stress level marker is selected from the group consisting of epinephrine, norepinephrine, cortisol, and any combination thereof.

The present disclosure provides for, and includes, a method of increasing tissue oxygenation in one or more vital organs of a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein the one or more vital organs are selected from the group consisting of the kidney, the liver, the lungs, the spleen, the heart, and any combination thereof.

### BRIEF DESCRIPTION OF THE DRAWINGS

The present disclosure is provided with reference to the accompanying drawings, wherein:

FIG. 1A displays changes in oxygen saturation (SO₂) of blood over time in an oxygen depletion device.

FIG. 1B displays changes in partial pressure of oxygen (pO₂) of blood over time in an oxygen depletion device.

FIG. 1C displays changes in partial pressure of carbon dioxide (pCO₂) of blood over time in an oxygen depletion device.

FIG. 1D displays changes in pH of blood over time in an oxygen depletion device.

FIG. 1E displays changes in viable red blood cells over time from fresh red blood cells (FRBCs), conventionally stored red blood cells (CRBCs), and hypoxically stored red blood cells (HRBCs) at 5 minutes and 24 hours after being intravenously administered in rats.

FIG. 1F displays changes in percentage of viable red blood cells from each group of blood (FRBCs, CRBCs, and HRBCs) at 5 minutes, 30 minutes, 1 hour, 2 hours, 4 hours, and 24 hours after intravenous administration in rats. (Significance: †=p<0.05 compared to 5 minute timepoint; *=p<0.05; **=p<0.01).

FIG. 2A displays differences in pH of blood between groups (FRBCs, CRBCs, and HRBCs) prior to administration to rats. (Significance: ****=<0.0001).

FIG. 2B displays differences in partial pressure of carbon dioxide (pCO₂) of blood between groups (FRBCs, CRBCs, and HRBCs) prior to administration to rats. (Significance: *=p<0.05; **=p<0.01; ***=p<0.001; ****=<0.0001).

FIG. 2C displays differences in total hemoglobin of blood between groups (FRBCs, CRBCs, and HRBCs) prior to administration to rats. A circle represents a data point that is an outlier. (Significance: *=p<0.05; **=p<0.01; ***=p<0.001; ****=<0.0001).

FIG. 2D displays differences in hematocrit of blood between groups (FRBCs, CRBCs, and HRBCs) prior to administration to rats. A circle represents a data point that is an outlier. (Significance: *=p<0.05; **=p<0.01; ***=p<0.001; ****=<0.0001).

FIG. 2E displays differences in partial pressure of oxygen (pO₂) of blood between groups (FRBCs, CRBCs, and HRBCs) prior to administration to rats. (Significance: *=p<0.05; **=p<0.01; ***=p<0.001; ****=<0.0001).

FIG. 2F displays differences in oxygen saturation (SO₂) of blood between groups (FRBCs, CRBCs, and HRBCs) prior to administration to rats. (Significance: *=p<0.05; **=p<0.01; ***=p<0.001; ****=<0.0001).

FIG. 2G displays differences in plasma hemoglobin of blood between groups (FRBCs, CRBCs, and HRBCs) prior to administration to rats. (Significance: *=p<0.05; **=p<0.01; ***=p<0.001; ****=<0.0001).

FIG. 2H displays differences in percentage of hemolysis in blood between groups (FRBCs, CRBCs, and HRBCs) prior to administration to rats. (Significance: *=p<0.05; **=p<0.01; ***=p<0.001; ****=<0.0001).

FIG. 3A displays changes in systolic blood pressure (SBP) in animals administered with different groups of blood (FRBCs, CRBCs, and HRBCs) at baseline, hemorrhagic shock, and after resuscitation. (Significance: *=p<0.05).

FIG. 3B displays changes in diastolic blood pressure (DBP) in animals administered with different groups of blood (FRBCs, CRBCs, and HRBCs) at baseline, hemorrhagic shock, and after resuscitation. (Significance: *=p<0.05).

FIG. 3C displays changes in mean arterial pressure (MAP) in animals administered with different groups of blood (FRBCs, CRBCs, and HRBCs) at baseline, hemorrhagic shock, and after resuscitation. (Significance: *=p<0.05).

FIG. 3D displays changes in heart rate (HR) in animals administered with different groups of blood (FRBCs, CRBCs, and HRBCs) at baseline, hemorrhagic shock, and after resuscitation.

FIG. 4A displays differences in levels of CXC motif chemokine ligand 1 (CXCL1) in the lungs of animals having a traumatic brain injury accompanied by hemorrhagic shock (TBI+HS) administered with different groups of blood (FRBCs, CRBCs, and HRBCs), compared to the CXCL1 level in the lungs of non-TBI+HS animals at baseline (Bl). (Significance: *=p<0.05).

FIG. 4B displays differences in levels of myeloperoxidase (MPO) in the lungs of TBI+HS animals administered with different groups of blood (FRBCs, CRBCs, and HRBCs), compared to the MPO level in the lungs of non-TBI+HS animals at baseline (BI). (Significance: *=p<0.05).

FIG. 4C displays differences in CD45+neutrophil content in the lungs of TBI+HS animals administered with different groups of blood (FRBCs, CRBCs, and HRBCs), compared to the neutrophil content in the lungs of non-TBI+HS animals at baseline (Bl). (Significance: *=p<0.05).

FIG. 5A displays differences in levels of CXC motif chemokine ligand 1 (CXCL1) in the livers of TBI+HS animals administered with different groups of blood (FRBCs, CRBCs, and HRBCs), compared to the CXCL1 level in the liver of non-TBI+HS animals at baseline (Bl). (Significance: *=p<0.05).

FIG. 5B displays differences in levels of aspartate aminotransferase (AST) in the livers of TBI+HS animals administered with different groups of blood (FRBCs, CRBCs, and HRBCs), compared to the AST level in the liver of non-TBI+HS animals at baseline (Bl). (Significance: *=p<0.05).

FIG. 5C displays differences in levels of alanine aminotransferase (ALT) in the livers of TBI+HS animals administered with different groups of blood (FRBCs, CRBCs, and HRBCs), compared to the ALT level in the liver of non-TBI+HS animals at baseline (Bl).

FIG. 6A displays differences in levels of interleukin 6 (IL-6) in the hearts of TBI+HS animals administered with different groups of blood (FRBCs, CRBCs, and HRBCs), compared to the IL-6 level in the heart of non-TBI+HS animals at baseline (Bl). (Significance: *=p<0.05).

FIG. 6B displays differences in levels of tumor necrosis factor alpha (TNF-α) in the hearts of TBI+HS animals administered with different groups of blood (FRBCs, CRBCs, and HRBCs), compared to the TNF-α level in the heart of non-TBI+HS animals at baseline (Bl). (Significance: *=p<0.05).

FIG. 6C displays differences in levels of monocyte chemoattractant protein-1 (MCP-1) in the hearts of TBI+HS animals administered with different groups of blood (FRBCs, CRBCs, and HRBCs), compared to the MCP1 level in the heart of non-TBI+HS animals at baseline (Bl). (Significance: *=p<0.05).

FIG. 6D displays differences in levels of troponin in the hearts of TBI+HS animals administered with different groups of blood (FRBCs, CRBCs, and HRBCs), compared to the troponin level in the heart of non-TBI+HS animals at baseline (Bl). (Significance: *=p<0.05).

FIG. 6E displays differences in levels of plasma C reactive protein (CRP) in the hearts of TBI+HS animals administered with different groups of blood (FRBCs, CRBCs, and HRBCs), compared to the plasma CRP level in the heart of non-TBI+HS animals at baseline (Bl). (Significance: *=p<0.05).

FIG. 6F displays differences in levels of plasma atrial natriuretic peptide (ANP) in the hearts of TBI+HS animals administered with different groups of blood (FRBCs, CRBCs, and HRBCs), compared to the plasma ANP level in the heart of non-TBI+HS animals at baseline (Bl). (Significance: *=p<0.05).

FIG. 7A displays differences in levels of superoxide dismutase (SOD) in tissue homogenates and plasma harvested from TBI+HS animals administered with different groups of blood (FRBCs, CRBCs, and HRBCs), compared to the SOD level in tissue homogenates and plasma harvested from non-TBI+HS animals at baseline (Bl). (Significance: *=p<0.05).

FIG. 7B displays differences in levels of catalase in tissue homogenates and plasma harvested from TBI+HS animals administered with different groups of blood (FRBCs, CRBCs, and HRBCs), compared to the catalase level in tissue homogenates and plasma harvested from non-TBI+HS animals at baseline (Bl). (Significance: *=p<0.05).

FIG. 7C displays differences in levels of thiobarbituric acid reactive substances (TBARS) in tissue homogenates and plasma harvested from TBI+HS animals administered with different groups of blood (FRBCs, CRBCs, and HRBCs), compared to the TBARS level in tissue homogenates and plasma harvested from non-TBI+HS animals at baseline (Bl). (Significance: *=p<0.05).

FIG. 7D displays differences in levels of 8-hydroxy-2′-deoxyguanosine (8—OHdG) in tissue homogenates and plasma harvested from TBI+HS animals administered with different groups of blood (FRBCs, CRBCs, and HRBCs), compared to the 8—OHdG level in tissue homogenates and plasma harvested from non-TBI+HS animals at baseline (Bl). (Significance: *=p<0.05).

FIG. 7E displays differences in levels of glutathione (GSH) in tissue homogenates and plasma harvested from TBI+HS animals administered with different groups of blood (FRBCs, CRBCs, and HRBCs), compared to the GSH level in tissue homogenates and plasma harvested from non-TBI+HS animals at baseline (Bl).

FIG. 8A displays differences in hematocrit (Hct) between each group of blood (FRBCs, CRBCs, and HRBCs) at 0, 10, 30, and 60 minutes during a recirculation challenge. (Significance: *=p<0.05 at each timepoint between groups; †=p<0.05 compared to 0 minutes within the same group).

FIG. 8B displays differences in levels of plasma hemoglobin (pHb) between each group of blood (FRBCs, CRBCs, and HRBCs) at 0, 10, 30, and 60 minutes during a recirculation challenge. (Significance: *=p<0.05 at each timepoint between groups; **=p<0.01 at each timepoint between groups; †=p<0.05 compared to 0 minutes within the same group).

FIG. 8C displays differences in hemolysis percentage between each group of blood (FRBCs, CRBCs, and HRBCs) at 0, 10, 30, and 60 minutes during a recirculation challenge. (Significance: *=p<0.05 at each timepoint between groups; †=p<0.05 compared to 0 minutes within the same group).

FIG. 8D displays differences in the normalized index of hemolysis (NIH) between each group of blood (FRBCs, CRBCs, and HRBCs) at 0, 10, 30, and 60 minutes during a recirculation challenge. (Significance: *=p<0.05 at each timepoint between groups; †=p<0.05 compared to 0 minutes within the same group).

FIG. 9A displays differences in neurological score between each group of animals (sham, FRBCs, CRBCs, and HRBCs) at baseline (BL), 2, 7, 14, 21, and 28 days after all steps are completed (i.e., after induction of TBI-HS resuscitation, and a 120-minute monitoring period).

FIG. 9B displays differences in the time (in seconds) taken to walk through a transverse beam between each group of animals (sham, FRBCs, CRBCs, and HRBCs), at baseline (BL), 2, 7, 14, 21, and 28 days after all steps are completed (i.e., after induction of TBI-HS, resuscitation, and an initial 120-minute monitoring period).

### DETAILED DESCRIPTION

Methods of the present disclosure provide for, and include, administering oxygen reduced blood having an oxygen saturation (SO₂) of 20% or less prior to and during storage to a patient with a traumatic brain injury (TBI) undergoing hemorrhagic shock. Also provided herein are methods for administering oxygen reduced blood having an oxygen saturation (SO₂) of between 15% and 20% prior to and during storage to a patient with a traumatic brain injury (TBI) undergoing hemorrhagic shock. Also provided herein are methods for administering oxygen reduced blood having an oxygen saturation (SO₂) of between 10% and 15% prior to and during storage to a patient with a traumatic brain injury (TBI) undergoing hemorrhagic shock. Also provided herein are methods for administering oxygen reduced blood having an oxygen saturation (SO₂) of between 5% and 10% prior to and during storage to a patient with a traumatic brain injury (TBI) undergoing hemorrhagic shock. Also provided herein are methods for administering oxygen reduced blood having an oxygen saturation (SO₂) of between 3% and 5% prior to and during storage to a patient with a traumatic brain injury (TBI) undergoing hemorrhagic shock. In aspects, oxygen reduced blood is administered by transfusion to a patient with a TBI undergoing hemorrhagic shock.

Methods of the present disclosure provide for, and include administering oxygen reduced blood that has an oxygen saturation (SO₂) of 20% or less prior to and during storage by transfusion to a traumatic brain injury (TBI) patient undergoing hemorrhagic shock. Also provided herein are methods comprising transfusing oxygen reduced blood that has an oxygen saturation of 20% or less prior to and during storage to a TBI patient having an increased risk of hemorrhagic shock due to surgery. Also provided herein are methods of administering oxygen reduced blood having an oxygen saturation of 20% or less comprising administering oxygen reduced blood having an oxygen saturation of 10% or less. Also provided herein are methods of administering oxygen reduced blood having an oxygen saturation of 20% or less comprising administering oxygen reduced blood having an oxygen saturation of 5% or less. Also provided herein are methods of administering oxygen reduced blood having an oxygen saturation of 20% or less comprising administering oxygen reduced blood having an initial oxygen saturation of 3% or less.

Methods of the present disclosure provide for, and include, administering oxygen reduced blood for the treatment of a traumatic brain injury (TBI) accompanied by hemorrhagic shock in a patient, where the oxygen reduced blood has an oxygen saturation of 20% or less prior to and during storage for a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of 15% or less after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of 10% or less after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of 5% or less after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of 3% or less after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of between 3 and 5% after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of between 5 and 10% after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of between 10 and 15% after a storage period of at least one week, at least two weeks, at least 3 weeks, at least 4 weeks, at least 5 weeks, or at least 6 weeks. Also provided herein are methods for administering oxygen reduced blood for the treatment of a TBI accompanied by hemorrhagic shock in a patient, where the oxygen reduced blood has an oxygen saturation of between 15 and 20% after a storage period of at least one week, at least two weeks, at least 3 weeks, at least 4 weeks, at least 5 weeks, or at least 6 weeks.

Methods of the present disclosure provide for, and include, administering to a traumatic brain injury (TBI) patient undergoing hemorrhagic shock oxygen reduced blood that has an oxygen saturation of 20% or less prior to and during storage. In an aspect, a TBI patient undergoing hemorrhagic shock suffers from a head trauma, a penetrating wound, blunt force trauma, injury due to a fall, or injury due to a car accident. In an aspect, a TBI patient undergoing hemorrhagic shock is a patient with a traumatic brain injury accompanied by hemorrhagic shock. In an aspect, the TBI accompanied by hemorrhagic shock is due to brain surgery, a penetrating wound in the head, blunt force trauma to the head, an injury to the head due to a fall, an injury to the head due to a car accident, or any combination thereof.

In an aspect of the present disclosure, a TBI patient undergoing hemorrhagic shock is a subject in need of oxygen reduced blood. In an aspect, a TBI patient undergoing hemorrhagic shock is in need of one or more units of blood. In an aspect, a TBI patient undergoing hemorrhagic shock is need of two or more units of blood. In an aspect, a TBI patient undergoing hemorrhagic shock is in need of three or more units of blood.

In an aspect of the present disclosure, a patient with a traumatic brain injury (TBI) accompanied by hemorrhagic shock is a TBI patient undergoing hemorrhagic shock. In an aspect, a patient with a traumatic brain injury (TBI) accompanied by hemorrhagic shock is undergoing hemorrhagic shock due to a head trauma, a penetrating wound, blunt force trauma, injury from a fall, or injury from a car accident. In an aspect, a TBI patient undergoing hemorrhagic shock is a patient with a class I hemorrhage. In an aspect, a TBI patient undergoing hemorrhagic shock is a patient with a class II hemorrhage. In an aspect, a TBI patient undergoing hemorrhagic shock is a patient with a class III hemorrhage. In an aspect, a TBI patient undergoing hemorrhagic shock is a patient with a class IV hemorrhage. In an aspect, a TBI patient undergoing hemorrhagic shock loses up to 15% of blood volume. In an aspect, a TBI patient undergoing hemorrhagic shock loses between 15 and 30% of blood volume. In an aspect, a TBI patient undergoing hemorrhagic shock loses between 30 and 40% of blood volume. In an aspect, a TBI patient undergoing hemorrhagic shock loses greater than 40% of blood volume.

The present disclosure provides for, and includes, a patient in need of administration with oxygen reduced blood exhibiting one or more negative parameters prior to administration, where the one or more negative parameters are selected from the group consisting of a reduced arterial oxygen saturation (SO₂), a reduced venous oxygen saturation (SO₂), an increased CXC motif chemokine ligand 1 (CXCL1) level, an increased myeloperoxidase (MPO) level, an increased lung neutrophil content, an increased alanine transaminase (ALT) level, an increased aspartate transaminase (AST) level, an increased alkaline phosphatase (ALP) level, an increased bilirubin level, an increased interleukin 6 (IL-6) level, an increased monocyte chemoattractant protein-1 (MCP1) level, an increased ferritin level, an increased albumin level, an increased albumin plus globulin (total marker) level, an increased tumor necrosis factor alpha (TNF-α) level, an increased troponin level, an increased plasma C reactive protein (CRP) level, an increased plasma atrial natriuretic peptide (ANP) level, a reduced interleukin 10 (IL-10) level, an increased epinephrine level, an increased norepinephrine level, an increased cortisol level, an increased urine creatinine level, an increased level of neutrophil gelatinase-associated lipocalin, an increased serum creatinine level, an increased blood urea nitrogen (BUN) level, a reduced tissue oxygen saturation in one or more vital organs, an increased blood lactate level, an increased blood glucose level, a reduced hematocrit, and a reduced cardiac output. In an aspect, a patient in need of administration with oxygen reduced blood is a TBI patient undergoing hemorrhagic shock having an increased CXC motif chemokine ligand 1 (CXCL1) level, an increased myeloperoxidase (MPO) level, an increased lung neutrophil content, or any combination thereof. In an aspect, a patient in need of administration with oxygen reduced blood is a TBI patient undergoing hemorrhagic shock having an increased alanine transaminase (ALT) level, an increased aspartate transaminase (AST) level, an increased alkaline phosphatase (ALP) level, an increased bilirubin level, an increased interleukin 6 (IL-6) level, an increased monocyte chemoattractant protein-1 (MCP1) level, an increased ferritin level, an increased CXC motif chemokine ligand 1 (CXCL1) level, an increased albumin level, an increased albumin plus globulin (total marker) level, or any combination thereof. In an aspect, a patient in need of administration with oxygen reduced blood is a TBI patient undergoing hemorrhagic shock having an increased interleukin 6 (IL-6) level, an increased CXC motif chemokine ligand 1 (CXCL1) level, an increased monocyte chemoattractant protein-1 (MCP1) level, an increased ferritin level, or any combination thereof. In an aspect, a patient in need of administration with oxygen reduced blood is a TBI patient undergoing hemorrhagic shock having an increased interleukin 6 (IL-6) level, an increased tumor necrosis factor alpha (TNF-α) level, an increased monocyte chemoattractant protein-1 (MCP1) level, an increased troponin level, an increased plasma C reactive protein (CRP) level, an increased plasma atrial natriuretic peptide (ANP) level, an increased ferritin level, or any combination thereof. In an aspect, a patient in need of administration with oxygen reduced blood is a TBI patient undergoing hemorrhagic shock having an increased interleukin 6 (IL-6) level, an increased CXC motif chemokine ligand 1 (CXCL1) level, a reduced interleukin 10 (IL-10) level, or any combination thereof. In an aspect, a patient in need of administration with oxygen reduced blood is a TBI patient undergoing hemorrhagic shock having an increased epinephrine level, an increased norepinephrine level, an increased cortisol level, or any combination thereof. In an aspect, a patient in need of administration with oxygen reduced blood is a TBI patient undergoing hemorrhagic shock having an increased urine creatinine level, an increased level of neutrophil gelatinase-associated lipocalin, an increased serum creatinine level, an increased blood urea nitrogen (BUN) level, or any combination thereof.

The present disclosure provides for, and includes, a patient in need of administration with oxygen reduced blood, where the patient exhibits reduced arterial oxygen saturation and increased blood lactate. The present disclosure provides for, and includes, a patient in need of administration with oxygen reduced blood, where the patient exhibits increased aspartate aminotransferase (AST), increased alanine aminotransferase (ALT), and increased blood urea nitrogen. The present disclosure provides for, and includes, a patient in need of administration with oxygen reduced blood, where the patient exhibits increased aspartate aminotransferase (AST), increased alanine aminotransferase (ALT), increased serum creatinine, and increased blood urea nitrogen. The present disclosure provides for, and includes, a patient in need of administration with oxygen reduced blood, where the patient exhibits increased levels of blood lactate and blood glucose. The present disclosure provides for, and includes, a patient in need of administration with oxygen reduced blood, where the patient exhibits increased urine neutrophil gelatinase-associated lipocalin (u-NGAL), increased serum creatinine, and increased blood urea nitrogen.

In an aspect, a patient in need of administration with oxygen reduced blood is a TBI patient undergoing hemorrhagic shock having a reduced hematocrit. In an aspect, a patient in need of administering with oxygen reduced blood is a TBI patient undergoing hemorrhagic shock having an increased blood lactate level. In an aspect, a patient in need of administering with oxygen reduced blood is a TBI patient undergoing hemorrhagic shock having an increased blood glucose level. In an aspect, a TBI patient undergoing hemorrhagic shock having an increased aspartate aminotransferase (AST) level. In an aspect, a patient in need of administering with oxygen reduced blood is a TBI patient undergoing hemorrhagic shock having an increased alanine aminotransferase (ALT) level. In an aspect, a patient in need of administering with oxygen reduced blood is a TBI patient undergoing hemorrhagic shock having an increased urine neutrophil gelatinase-associated lipocalin (u-NGAL) level. In an aspect, a patient in need of administration with oxygen reduced blood is a TBI patient undergoing hemorrhagic shock having an increased serum creatinine level. In an aspect, a patient in need of administration with oxygen reduced blood is a TBI patient undergoing hemorrhagic shock having an increased blood urea nitrogen level.

In an aspect, oxygen reduced blood for use in administration to a traumatic brain injury (TBI) patient undergoing hemorrhagic shock in need thereof has an oxygen saturation of 20% or less. In an aspect, oxygen reduced blood has an oxygen saturation of 10% or less. In an aspect, oxygen reduced blood has an oxygen saturation of 5% or less. In an aspect, oxygen reduced blood has an oxygen saturation of 3% or less.

In an aspect, the oxygen reduced blood for use in administration to a traumatic brain injury (TBI) patient undergoing hemorrhagic shock in need thereof has a partial pressure of carbon dioxide (pCO₂) at 37 °C of between 10 and 40 mmHg. In an aspect, oxygen reduced blood has a pCO₂ at 37 °C of between 10 and 30 mmHg. In an aspect, oxygen reduced blood has a pCO₂ at 37 °C of between 10 and 20 mmHg. In an aspect, oxygen reduced blood has a pCO₂ at 37 °C of between 10 and 15 mmHg. In an aspect, oxygen reduced blood has a pCO₂ at 37 °C of less than 10 mmHg.

In an aspect, oxygen reduced blood for use in administration to a traumatic brain injury (TBI) patient undergoing hemorrhagic shock in need thereof has an oxygen saturation of 20% or less and is stored for less than 2 days. In an aspect, oxygen reduced blood has an oxygen saturation of 20% or less and is stored for less than 7 days. In an aspect, oxygen reduced blood has an oxygen saturation of 20% or less and is stored for less than 14 days. In an aspect, oxygen reduced blood has an oxygen saturation of 20% or less and is stored for less than 21 days. In an aspect, oxygen reduced blood for use in transfusion therapy of a trauma patient in need thereof has an oxygen saturation of 20% or less and is stored for less than 28 days. In an aspect, oxygen reduced blood has an oxygen saturation of 20% or less and is stored for less than 35 days. In an aspect, oxygen reduced blood has an oxygen saturation of 20% or less and is stored for less than 42 days. In an aspect, oxygen reduced blood has an oxygen saturation of 20% or less and is stored for less than 45 days. In an aspect, oxygen reduced blood has an oxygen saturation of 20% or less during storage.

Suitable blood for use in the methods according to the present disclosure for treating a traumatic brain injury (TBI) accompanied by hemorrhagic shock in a patient is oxygen reduced blood having an anticoagulant. In an aspect, oxygen reduced red blood cells is stored for up to 3 weeks to produce oxygen reduced blood. In an aspect, oxygen reduced blood may comprise an additive solution. Suitable additive solutions according to the present disclosure include additive solution 1 (AS-1), additive solution 3 (AS-3; Nutricel®), additive solution 5 (AS-5), saline-adenine-glucose-mannitol (SAGM), phosphate-adenine-glucose-guanosine-saline-mannitol (PAGG-SM), phosphate-adenine-glucose-guanosine-gluconate-mannitol (PAGG-GM), mannitol-adenine-phosphate (MAP), additive solution 7 (AS-7), erythro-sol 5 (ESOL-5), experimental additive solution 61 (EAS61), oxygen free additive solution 1 (OFAS1), oxygen free additive solution 3 (OFAS3), and any combinations thereof. In an aspect, the additive solution is added at the time of component separation. In an aspect, the additive solution is AS-1. In another aspect, the additive solution is AS-3. In other aspects, the additive solution is SAGM.

In an aspect of the present disclosure, a patient treated according to the methods disclosed herein has a traumatic brain injury accompanied by hemorrhagic shock due to a head trauma, a penetrating wound, blunt force trauma, injury from a fall, or injury from a car accident. In aspects of the present disclosure, a TBI patient undergoing hemorrhagic shock exhibits a Glasgow Coma Scale (GCS) score of 12 or less. In an aspect, a TBI patient undergoing hemorrhagic shock exhibits a GCS score of between 3 and 12. In an aspect, a TBI patient undergoing hemorrhagic shock exhibits a GCS score of between 9 and 12. In an aspect, a TBI patient undergoing hemorrhagic shock exhibits a GCS score of between 3 and 8.

In aspects of the present disclosure, a TBI patient undergoing hemorrhagic shock has a shock index (SI) of greater than 0.9. In an aspect, a TBI patient undergoing hemorrhagic shock has a shock index (SI) of greater than 1.0.

Methods of the present disclosure provide for, and include, increasing the arterial oxygen saturation (SO₂) in a TBI patient undergoing hemorrhagic shock, comprising administering to the patient oxygen reduced blood having an oxygen saturation (SO₂) of 20% or less prior to and during storage. In an aspect, the arterial SO₂ of the TBI patient undergoing hemorrhagic shock is increased by at least 10% after the administering. In an aspect, the arterial SO₂ of the TBI patient undergoing hemorrhagic shock is increased by at least 20% after the administering. In an aspect, the arterial SO₂ of the TBI patient undergoing hemorrhagic shock is increased by between 10% to 20% after the administering. In an aspect, the arterial SO₂ of the TBI patient undergoing hemorrhagic shock is increased by between 10% to 30% after the administering. In an aspect, the arterial SO₂ of the TBI patient undergoing hemorrhagic shock is increased by between 10% to 40% after the administering. In an aspect, the arterial SO₂ of the TBI patient undergoing hemorrhagic shock is increased by between 10% to 50% after the administering. In an aspect, the arterial SO₂ of the TBI patient undergoing hemorrhagic shock is increased by between 20% to 30% after the administering. In an aspect, the arterial SO₂ of the TBI patient undergoing hemorrhagic shock is increased by between 20% to 40% after the administering. In an aspect, the arterial SO₂ of the TBI patient undergoing hemorrhagic shock is increased by between 20% to 50% after the administering. In an aspect, the arterial SO₂ of the TBI patient undergoing hemorrhagic shock is increased by between 30% to 40% after the administering. In an aspect, the arterial SO₂ of the TBI patient undergoing hemorrhagic shock is increased by between 30% to 50% after the administering. In an aspect, the arterial SO₂ of the TBI patient undergoing hemorrhagic shock is increased by between 40% to 50% after the administering. In an aspect, the arterial SO₂ of the TBI patient undergoing hemorrhagic shock is increased by at least 10%, 20%, 30%, 40%, or 50% after the administering.

In an aspect of the present disclosure, the arterial oxygen saturation (SO₂) of a TBI patient undergoing hemorrhagic shock is increased by at least 1.5 fold after administering oxygen reduced blood having an oxygen saturation (SO₂) of 20% or less prior to and during storage to the patient. In an aspect, the arterial SO₂ of the TBI patient undergoing hemorrhagic shock is increased by at least 2 fold after the administering. In an aspect, the arterial SO₂ of the TBI patient undergoing hemorrhagic shock is increased by at least 10 mmHg, at least 20 mmHg, at least 30 mmHg, at least 40 mmHg, at least 50 mmHg, or at least 60 mmHg after the administering. In an aspect, the arterial SO₂ of the TBI patient undergoing hemorrhagic shock is increased by between 20 and 50 mmHg after the administering. In an aspect, the arterial SO₂ of the TBI patient undergoing hemorrhagic shock is increased by between 30 and 50 mmHg after the administering.

Methods of the present disclosure provide for, and include, increasing the arterial oxygen saturation (SO₂) of a TBI patient undergoing hemorrhagic shock, comprising administering oxygen reduced blood having an oxygen saturation (SO₂) of 20% or less prior to and during storage to the patient, compared to the SO₂ of a TBI patient undergoing hemorrhagic shock having been administered the same amount of non-oxygen reduced conventionally stored blood. In an aspect, the arterial SO₂ of the TBI patient undergoing hemorrhagic shock is increased by between 10% to 20% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 10% to 30% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 10% to 40% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 10% to 50% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 20% to 30% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 20% to 40% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 20% to 50% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 30% to 40% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by between 30% to 50% after the administering, compared to a TBI patient undergoing hemorrhagic shock having been administered the same amount of non-oxygen reduced conventionally stored blood. In an aspect, the arterial SO₂ of a TBI patient undergoing hemorrhagic shock is increased by between 40% to 50% after the administering, compared to a TBI patient undergoing hemorrhagic shock having been administered the same amount of non-oxygen reduced conventionally stored blood. In an aspect, the arterial SO₂ of the TBI patient undergoing hemorrhagic shock is increased by at least 10%, 20%, 30%, 40%, or 50% after the administering, compared to a TBI patient undergoing hemorrhagic shock having been administered the same amount of non-oxygen reduced conventionally stored blood.

Methods of the present disclosure provide for, and include, increasing the arterial oxygen saturation (SO₂) of a TBI patient undergoing hemorrhagic shock to between about 70 and about 110 millimeters of mercury (mmHg), comprising administering oxygen reduced blood having an oxygen saturation (SO₂) of 20% or less prior to and during storage to the patient. In an aspect, the arterial SO₂ of the TBI patient undergoing hemorrhagic shock is increased to between about 80 and about 110 mmHg after the administering. In an aspect, the arterial SO₂ of the TBI patient undergoing hemorrhagic shock is increased to between about 90 and about 110 mmHg after administering. In an aspect, the arterial SO₂ of the TBI patient undergoing hemorrhagic shock is increased to between about 75 and about 100 mmHg after administering.

Methods of the present disclosure provide for, and include, increasing the venous oxygen saturation (SO₂) in a TBI patient undergoing hemorrhagic shock, comprising administering to the TBI patient undergoing hemorrhagic shock oxygen reduced blood having an oxygen saturation (SO₂) of 20% or less prior to and during storage. In an aspect, the venous SO₂ of the TBI patient undergoing hemorrhagic shock is increased by at least 10% after the administering. In an aspect, the venous SO₂ of the TBI patient undergoing hemorrhagic shock is increased by at least 20% after the administering. In an aspect, the venous SO₂ of the TBI patient undergoing hemorrhagic shock is increased by between 10% to 20% after the administering. In an aspect, the venous SO₂ of the TBI patient undergoing hemorrhagic shock is increased by between 10% to 30% after the administering. In an aspect, the venous SO₂ of the TBI patient undergoing hemorrhagic shock is increased by between 10% to 40% after the administering. In an aspect, the venous SO₂ of the TBI patient undergoing hemorrhagic shock is increased by between 10% to 50% after the administering. In an aspect, the venous SO₂ of the TBI patient undergoing hemorrhagic shock is increased by between 20% to 30% after the administering. In an aspect, the venous SO₂ of the TBI patient undergoing hemorrhagic shock is increased by between 20% to 40% after the administering. In an aspect, the venous SO₂ of the TBI patient undergoing hemorrhagic shock is increased by between 20% to 50% after the administering. In an aspect, the venous SO₂ of the TBI patient undergoing hemorrhagic shock is increased by between 30% to 40% after the administering. In an aspect, the venous SO₂ of the TBI patient undergoing hemorrhagic shock is increased by between 30% to 50% after the administering. In an aspect, the venous SO₂ of the TBI patient undergoing hemorrhagic shock is increased by between 40% to 50% after the administering. In an aspect, the venous SO₂ of the TBI patient undergoing hemorrhagic shock is increased by at least 10%, 20%, 30%, 40%, or 50% after the administering.

In an aspect of the present disclosure, the venous oxygen saturation (SO₂) of a TBI patient undergoing hemorrhagic shock is increased by at least 1.5 fold after administering oxygen reduced blood having an oxygen saturation (SO₂) of 20% or less prior to and during storage. In an aspect, the venous SO₂ of the TBI patient undergoing hemorrhagic shock is increased by at least 2 fold after the administering. In an aspect, the venous SO₂ of the TBI patient undergoing hemorrhagic shock is increased by at least 10 mmHg, at least 20 mmHg, at least 30 mmHg, at least 40 mmHg, at least 50 mmHg, or at least 60 mmHg after the administering. In an aspect, the venous SO₂ of the TBI patient undergoing hemorrhagic shock is increased by between 20 and 50 mmHg after the administering. In a further aspect, the venous SO₂ of the TBI patient undergoing hemorrhagic shock is increased by between 30 and 50 mmHg after the administering.

Methods of the present disclosure provide for, and include, increasing the venous oxygen saturation (SO₂) of a TBI patient undergoing hemorrhagic shock, comprising administering a TBI patient undergoing hemorrhagic shock with oxygen reduced blood having an oxygen saturation (SO₂) of 20% or less prior to and during storage, compared to the venous SO₂ of a TBI patient undergoing hemorrhagic shock having been administered the same amount of non-oxygen reduced conventionally stored blood. In an aspect, the venous SO₂ of the TBI patient undergoing hemorrhagic shock is increased at least 10% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased at least 20% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 10% to 20% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 10% to 30% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 10% to 40% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 10% to 50% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 20% to 30% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 20% to 40% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 20% to 50% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 30% to 40% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 30% to 50% after the administering, compared to a TBI patient undergoing hemorrhagic shock having been administered the same amount of non-oxygen reduced conventionally stored blood. In an aspect, the venous SO₂ of the TBI patient undergoing hemorrhagic shock is increased by between 40% to 50% after the administering, compared to a TBI patient undergoing hemorrhagic shock having been administered the same amount of non-oxygen reduced conventionally stored blood. In an aspect, the venous SO₂ In an aspect, the venous SO₂ of the TBI patient undergoing hemorrhagic shock is increased by at least 10%, 20%, 30%, 40%, or 50% after the administering, compared to a TBI patient undergoing hemorrhagic shock having been administered the same amount of non-oxygen reduced conventionally stored blood.

Methods of the present disclosure provide for, and include, increasing the venous oxygen saturation (SO₂) of a TBI patient undergoing hemorrhagic shock to between about 70 and about 110 millimeters of mercury (mmHg), comprising administering a TBI patient undergoing hemorrhagic shock with oxygen reduced blood having an oxygen saturation (SO₂) of 20% or less prior to and during storage. In an aspect, the venous SO₂ of the TBI patient undergoing hemorrhagic shock is increased to between about 80 and about 110 mmHg after the administering. In an aspect, the venous SO₂ of the TBI patient undergoing hemorrhagic shock is increased to between about 90 and about 110 mmHg after administering. In an aspect, the venous SO₂ of the TBI patient undergoing hemorrhagic shock is increased to between about 75 and about 100 mmHg after administering.

The present disclosure provides for, and includes, a TBI patient undergoing hemorrhagic shock administered oxygen reduced blood having an oxygen saturation (SO₂) of 20% or less prior to and during storage who exhibits an improved parameter after the administering. In an aspect, a TBI patient undergoing hemorrhagic shock who is administered oxygen reduced blood according to the present disclosure exhibits an improved parameter after the administering, compared to a TBI patient undergoing hemorrhagic shock having been administered the same the same amount of non-oxygen reduced conventionally stored blood. In an aspect, the improved parameter is improved pulmonary function. In an aspect, the improved pulmonary function is reduced levels of CXC motif chemokine ligand 1 (CXCL1), myeloperoxidase (MPO), lung neutrophils, or any combination thereof. In an aspect, the improved parameter is improved hepatic function. In an aspect, the improved hepatic function is reduced levels of alanine transaminase (ALT), aspartate transaminase (AST), alkaline phosphatase (ALP), bilirubin, interleukin 6 (IL-6), CXC motif chemokine ligand 1 (CXCL1), monocyte chemoattractant protein-1 (MCP1), ferritin, albumin, albumin plus globulin (total protein), or any combination thereof. In an aspect, the improved parameter is improved splenic function. In an aspect, the improved splenic function is reduced levels of interleukin 6 (IL-6), CXC motif chemokine ligand 1 (CXCL1), monocyte chemoattractant protein-1 (MCP1), ferritin, or any combination thereof. In an aspect, the improved parameter is improved cardiac function. In an aspect, the improved cardiac function is reduced levels of interleukin 6 (IL-6), tumor necrosis factor alpha (TNF-α), monocyte chemoattractant protein-1 (MCP1), troponin, plasma C reactive protein (CRP), plasma atrial natriuretic peptide (ANP), ferritin, or any combination thereof. In an aspect, the improved parameter is an improved cardiac parameter. In an aspect, the improved cardiac parameter is an increased stroke volume (SV), an increased stroke work (SW), an increased cardiac output (CO), an increased contractility, an increased arterial elastance, an increased internal energy utilization (IEU), a reduced systolic volume (Ves), an increased end diastolic volume (Ved), a reduced systemic vascular resistance (SVR), or any combination thereof. In an aspect, the improved parameter is improved renal function. In an aspect, the improved renal function is reduced levels of neutrophil gelatinase-associated lipocalin (NGAL), urine creatinine, serum creatinine, blood urea nitrogen (BUN), or any combination thereof.

Methods of the present disclosure provide for, and include, reducing the level of CXC motif chemokine ligand 1 (CXCL1) in a TBI patient undergoing hemorrhagic shock, comprising administering to the TBI patient undergoing hemorrhagic shock oxygen reduced blood having an oxygen saturation (SO₂) of 20% or less prior to and during storage. In an aspect, the CXCL1 level in the TBI patient undergoing hemorrhagic shock is reduced by between 10% to 20% after the administering. In an aspect, the CXCL1 level in the TBI patient undergoing hemorrhagic shock is reduced by between 10% to 30% after the administering. In an aspect, the CXCL1 level in the TBI patient undergoing hemorrhagic shock is reduced by between 10% to 40% after the administering. In an aspect, the CXCL1 level in the TBI patient undergoing hemorrhagic shock is reduced by between 10% to 50% after the administering. In an aspect, the CXCL1 level in the TBI patient undergoing hemorrhagic shock is reduced by between 20% to 30% after the administering. In an aspect, the CXCL1 level in the TBI patient undergoing hemorrhagic shock is reduced by between 20% to 40% after the administering. In an aspect, the CXCL1 level in the TBI patient undergoing hemorrhagic shock is reduced by between 20% to 50% after the administering. In an aspect, the CXCL1 level in the TBI patient undergoing hemorrhagic shock is reduced by between 30% to 40% after the administering. In an aspect, the CXCL1 level in the TBI patient undergoing hemorrhagic shock is reduced by between 30% to 50% after the administering. In an aspect, the CXCL1 level in the TBI patient undergoing hemorrhagic shock is reduced by between 40% to 50% after the administering. In an aspect, the CXCL1 level in the TBI patient undergoing hemorrhagic shock is reduced by at least 10%, 20%, 30%, 40%, or 50% after the administering.

In an aspect, the level of CXC motif chemokine ligand 1 (CXCL1) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the CXCL1 level in the TBI patient undergoing hemorrhagic shock is reduced by between 1.5 and 3 fold after the administering. In an aspect, the CXCL1 level in the TBI patient undergoing hemorrhagic shock is reduced by between 1.5 and 5 fold after the administering. In an aspect, the CXCL1 level in the TBI patient undergoing hemorrhagic shock is reduced by between 1.5 and 10 fold after the administering. In an aspect, the CXCL1 level in the TBI patient undergoing hemorrhagic shock is reduced by between 3 and 5 fold after the administering. In an aspect, the CXCL1 level in the TBI patient undergoing hemorrhagic shock is reduced by between 3 and 10 fold after the administering. In an aspect, the CXCL1 level in the TBI patient undergoing hemorrhagic shock is reduced by between 5 and 10 fold after the administering.

Methods of the present disclosure provide for, and include, reducing the level of CXC motif chemokine ligand 1 (CXCL1) in a TBI patient undergoing hemorrhagic shock, comprising administering to the patient oxygen reduced blood having an oxygen saturation (SO₂) of 20% or less prior to and during storage, compared to the level of CXCL1 in a TBI patient undergoing hemorrhagic shock having been administered the same amount of non-oxygen reduced conventionally stored blood. In an aspect, the CXCL1 level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the CXCL1 level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the CXCL1 level in the TBI patient undergoing hemorrhagic shock is reduced by between 30% to 60% after the administering, compared to a TBI patient undergoing hemorrhagic shock having been administered the same amount of non-oxygen reduced conventionally stored blood. In an aspect, the CXCL1 level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the CXCL1 level in the TBI patient undergoing hemorrhagic shock is reduced by between 40% to 60% after the administering, compared to a TBI patient undergoing hemorrhagic shock having been administered the same amount of non-oxygen reduced conventionally stored blood. In an aspect, the CXCL1 level in the TBI patient undergoing hemorrhagic shock is reduced by between 50% to 60% after the administering, compared to a TBI patient undergoing hemorrhagic shock having been administered the same amount of non-oxygen reduced conventionally stored blood. In an aspect, the CXCL1 level in the TBI patient undergoing hemorrhagic shock is reduced by at least 30%, 40%, 50%, or 60% after the administering, compared to a TBI patient undergoing hemorrhagic shock having been administered the same amount of non-oxygen reduced conventionally stored blood.

Methods of the present disclosure provide for, and include, reducing the level of CXC motif chemokine ligand 1 (CXCL1) in a TBI patient undergoing hemorrhagic shock to between about 30 and about 90 picograms per milliliter (pg/mL) of serum, comprising administering to the patient oxygen reduced blood having an oxygen saturation (SO₂) of 20% or less prior to and during storage. In an aspect, the CXCL1 level in the TBI patient undergoing hemorrhagic shock is reduced to between about 30 and about 50 pg/mL of serum after the administering. In an aspect, the CXCL1 level in the TBI patient undergoing hemorrhagic shock is reduced to between about 30 and about 70 pg/mL of serum after the administering. In an aspect, the CXCL1 level in the TBI patient undergoing hemorrhagic shock is reduced to between about 50 and about 90 pg/mL of serum after the administering. In an aspect, the CXCL1 level in the TBI patient undergoing hemorrhagic shock is reduced to between about 70 and about 90 pg/mL of serum after the administering.

Methods of the present disclosure provide for, and include, reducing the level of myeloperoxidase (MPO) in a TBI patient undergoing hemorrhagic shock, comprising administering to the patient oxygen reduced blood having an oxygen saturation (SO₂) of 20% or less prior to and during storage. In an aspect, the MPO level in the TBI patient undergoing hemorrhagic shock is reduced by between 10% to 20% after the administering. In an aspect, the MPO level in the TBI patient undergoing hemorrhagic shock is reduced by between 10% to 30% after the administering. In an aspect, the MPO level in the TBI patient undergoing hemorrhagic shock is reduced by between 10% to 40% after the administering. In an aspect, the MPO level in the TBI patient undergoing hemorrhagic shock is reduced by between 10% to 50% after the administering. In an aspect, the MPO level in the TBI patient undergoing hemorrhagic shock is reduced by between 20% to 30% after the administering. In an aspect, the MPO level in the TBI patient undergoing hemorrhagic shock is reduced by between 20% to 40% after the administering. In an aspect, the MPO level in the TBI patient undergoing hemorrhagic shock is reduced by between 20% to 50% after the administering. In an aspect, the MPO level in the TBI patient undergoing hemorrhagic shock is reduced by between 30% to 40% after the administering. In an aspect, the MPO level in the TBI patient undergoing hemorrhagic shock is reduced by between 30% to 50% after the administering. In an aspect, the MPO level in the TBI patient undergoing hemorrhagic shock is reduced by between 40% to 50% after the administering. In an aspect, the MPO level in the TBI patient undergoing hemorrhagic shock is reduced by at least 10%, 20%, 30%, 40%, or 50% after the administering.

In an aspect of the present disclosure, the level of the level of myeloperoxidase (MPO) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the MPO level in the TBI patient undergoing hemorrhagic shock is reduced by between 1.5 and 3 fold after the administering. In an aspect, the MPO level in the TBI patient undergoing hemorrhagic shock is reduced by between 1.5 and 5 fold after the administering. In an aspect, the MPO level in the TBI patient undergoing hemorrhagic shock is reduced by between 1.5 and 10 fold after the administering. In an aspect, the MPO level in the TBI patient undergoing hemorrhagic shock is reduced by between 3 and 5 fold after the administering. In an aspect, the MPO level in the TBI patient undergoing hemorrhagic shock is reduced by between 3 and 10 fold after the administering. In an aspect, the MPO level in the TBI patient undergoing hemorrhagic shock is reduced by between 5 and 10 fold after the administering.

Methods of the present disclosure provide for, and include, reducing the level of myeloperoxidase (MPO) in a TBI patient undergoing hemorrhagic shock, comprising administering to the patient oxygen reduced blood having an oxygen saturation (SO₂) of 20% or less prior to and during storage, compared to the level of MPO in a TBI patient undergoing hemorrhagic shock having been administered the same amount of non-oxygen reduced conventionally stored blood. In an aspect, the MPO level in the TBI patient undergoing hemorrhagic shock is reduced by between 10% to 20% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 10% to 30% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 10% to 40% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 10% to 50% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 20% to 30% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 20% to 40% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 20% to 50% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 30% to 40% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 30% to 50% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by between 40% to 50% after administering, compared to a TBI patient undergoing hemorrhagic shock having been administered the same amount of non-oxygen reduced conventionally stored blood. In an aspect, the MPO level in the TBI patient undergoing hemorrhagic shock is reduced by at least 10%, 20%, 30%, 40%, or 50% after administering, compared to a TBI patient undergoing hemorrhagic shock having been administered the same amount of non-oxygen reduced conventionally stored blood.

Methods of the present disclosure provide for, and include, reducing the level of myeloperoxidase (MPO) in a TBI patient undergoing hemorrhagic shock to between about 10 and about 240 picograms per milliliter (pg/mL) of serum, comprising administering to the patient oxygen reduced blood having an oxygen saturation (SO₂) of 20% or less prior to and during storage. In an aspect, the MPO level in the TBI patient undergoing hemorrhagic shock is reduced to between about 30 and about 240 pg/mL of serum after the administering. In an aspect, the MPO level in the TBI patient undergoing hemorrhagic shock is reduced to between about 50 and about 240 pg/mL of serum after the administering. In an aspect, the MPO level in the TBI patient undergoing hemorrhagic shock is reduced to between about 100 and about 240 pg/mL of serum after the administering. In an aspect, the MPO level in the TBI patient undergoing hemorrhagic shock is reduced to between about 150 and about 240 pg/mL of serum after the administering. In an aspect, the MPO level in the TBI patient undergoing hemorrhagic shock is reduced to between about 180 and about 220 pg/mL of serum after the administering.

Methods of the present disclosure provide for, and include, reducing lung neutrophil content of a TBI patient undergoing hemorrhagic shock, comprising administering to the patient oxygen reduced blood having an oxygen saturation (SO₂) of 20% or less prior to and during storage. In an aspect, the lung neutrophil content in the TBI patient undergoing hemorrhagic shock is reduced by between 10% to 20% after the administering. In an aspect, the lung neutrophil content in the TBI patient undergoing hemorrhagic shock is reduced by between 10% to 30% after the administering. In an aspect, the lung neutrophil content in the TBI patient undergoing hemorrhagic shock is reduced by between 10% to 40% after the administering. In an aspect, the lung neutrophil content in the TBI patient undergoing hemorrhagic shock is reduced by between 10% to 50% after the administering. In an aspect, the lung neutrophil content in the TBI patient undergoing hemorrhagic shock is reduced by between 10% to 60% after the administering. In an aspect, the lung neutrophil content in the TBI patient undergoing hemorrhagic shock is reduced by between 10% to 70% after the administering. In an aspect, the lung neutrophil content in the TBI patient undergoing hemorrhagic shock is reduced by between 10% to 80% after the administering. In an aspect, the lung neutrophil content in the TBI patient undergoing hemorrhagic shock is reduced by between 20% to 30% after the administering. In an aspect, the lung neutrophil content in the TBI patient undergoing hemorrhagic shock is reduced by between 20% to 40% after the administering. In an aspect, the lung neutrophil content in the TBI patient undergoing hemorrhagic shock is reduced by between 20% to 50% after the administering. In an aspect, the lung neutrophil content in the TBI patient undergoing hemorrhagic shock is reduced by between 20% to 60% after the administering. In an aspect, the lung neutrophil content in the TBI patient undergoing hemorrhagic shock is reduced by between 20% to 70% after the administering. In an aspect, the lung neutrophil content in the TBI patient undergoing hemorrhagic shock is reduced by between 20% to 80% after the administering. In an aspect, the lung neutrophil content in the TBI patient undergoing hemorrhagic shock is reduced by between 30% to 40% after the administering. In an aspect, the lung neutrophil content in the TBI patient undergoing hemorrhagic shock is reduced by between 30% to 50% after the administering. In an aspect, the lung neutrophil content in the TBI patient undergoing hemorrhagic shock is reduced by between 30% to 60% after the administering. In an aspect, the lung neutrophil content in the TBI patient undergoing hemorrhagic shock is reduced by between 30% to 70% after the administering. In an aspect, the lung neutrophil content in the TBI patient undergoing hemorrhagic shock is reduced by between 30% to 80% after the administering. In an aspect, the lung neutrophil content in the TBI patient undergoing hemorrhagic shock is reduced by between 40% to 50% after the administering. In an aspect, the lung neutrophil content in the TBI patient undergoing hemorrhagic shock is reduced by between 40% to 60% after the administering. In an aspect, the lung neutrophil content in the TBI patient undergoing hemorrhagic shock is reduced by between 40% to 70% after the administering. In an aspect, the lung neutrophil content in the TBI patient undergoing hemorrhagic shock is reduced by between 40% to 80% after the administering. In an aspect, the lung neutrophil content in the TBI patient undergoing hemorrhagic shock is reduced by between 50% to 60% after the administering. In an aspect, the lung neutrophil content in the TBI patient undergoing hemorrhagic shock is reduced by between 50% to 70% after the administering. In an aspect, the lung neutrophil content in the TBI patient undergoing hemorrhagic shock is reduced by between 50% to 80% after the administering. In an aspect, the lung neutrophil content in the TBI patient undergoing hemorrhagic shock is reduced by between 60% to 70% after the administering. In an aspect, the lung neutrophil content in the TBI patient undergoing hemorrhagic shock is reduced by between 60% to 80% after the administering. In an aspect, the lung neutrophil content in the TBI patient undergoing hemorrhagic shock is reduced by between 70% to 80% after the administering. In an aspect, the lung neutrophil content in the TBI patient undergoing hemorrhagic shock is reduced by at least 10%, 20%, 30%, 40%, 50%, 60%, 70%, or 80% after the administering.

In an aspect, the lung neutrophil content of the TBI patient undergoing hemorrhagic shock is reduced by between 1.5 and 10 fold after the administering. In an aspect, the lung neutrophil content of the TBI patient undergoing hemorrhagic shock is reduced by between 1.5 and 3 fold after the administering. In an aspect, the lung neutrophil content of the TBI patient undergoing hemorrhagic shock is reduced by between 1.5 and 5 fold after the administering. In an aspect, the lung neutrophil content of the TBI patient undergoing hemorrhagic shock is reduced by between 1.5 and 10 fold after the administering. In an aspect, the lung neutrophil content of the TBI patient undergoing hemorrhagic shock is reduced by between 3 and 5 fold after the administering. In an aspect, the lung neutrophil content of the TBI patient undergoing hemorrhagic shock is reduced by between 3 and 10 fold after the administering. In an aspect, the lung neutrophil content of the TBI patient undergoing hemorrhagic shock is reduced by between 5 and 10 fold after the administering.

Methods of the present disclosure provide for, and include, reducing the level of lung neutrophil content of a TBI patient undergoing hemorrhagic shock, comprising administering to the patient oxygen reduced blood having an oxygen saturation (SO₂) of 20% or less prior to and during storage, compared to the level of lung neutrophil content of a TBI patient undergoing hemorrhagic shock having been administered the same amount of non-oxygen reduced conventionally stored blood. In an aspect, the lung neutrophil content of the TBI patient undergoing hemorrhagic shock is reduced by between 10% to 20% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 10% to 30% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 10% to 40% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 10% to 50% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 20% to 30% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 20% to 40% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 20% to 50% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 30% to 40% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 30% to 50% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by between 40% to 50% after administering, compared to a TBI patient undergoing hemorrhagic shock having been administered the same amount of non-oxygen reduced conventionally stored blood. In an aspect, the lung neutrophil content of the TBI patient undergoing hemorrhagic shock is reduced by at least 10%, 20%, 30%, 40%, or 50% after administering, compared to a TBI patient undergoing hemorrhagic shock having been administered the same amount of non-oxygen reduced conventionally stored blood.

Methods of the present disclosure provide for, and include, reducing lung neutrophil content of a TBI patient undergoing hemorrhagic shock to between 30% and 80%, comprising administering to the patient oxygen reduced blood having an oxygen saturation (SO₂) of 20% or less prior to and during storage. In an aspect, the lung neutrophil content of the TBI patient undergoing hemorrhagic shock is reduced to between 30% to 45% after the administering. In an aspect, the lung neutrophil content of the TBI patient undergoing hemorrhagic shock is reduced to between 30% to 60% after the administering. In an aspect, the lung neutrophil content of the TBI patient undergoing hemorrhagic shock is reduced to between 40% to 70% after the administering. In an aspect, the lung neutrophil content of the TBI patient undergoing hemorrhagic shock is reduced to between 45% to 60% after the administering. In an aspect, the lung neutrophil content of the TBI patient undergoing hemorrhagic shock is reduced to between 45% to 80% after the administering. In an aspect, the lung neutrophil content of the TBI patient undergoing hemorrhagic shock is reduced to between 60% to 80% after the administering.

Methods of the present disclosure provide for, and include, reducing the level of alkaline phosphatase (ALP) in a TBI patient undergoing hemorrhagic shock, comprising administering to the patient oxygen reduced blood having an oxygen saturation (SO₂) of 20% or less prior to and during storage. In an aspect, the ALP level in the TBI patient undergoing hemorrhagic shock is reduced by between 10% to 20% after the administering. In an aspect, the ALP level in the TBI patient undergoing hemorrhagic shock is reduced by between 10% to 30% after the administering. In an aspect, the ALP level in the TBI patient undergoing hemorrhagic shock is reduced by between 10% to 40% after the administering. In an aspect, the ALP level in the TBI patient undergoing hemorrhagic shock is reduced by between 10% to 50% after the administering. In an aspect, the ALP level in the TBI patient undergoing hemorrhagic shock is reduced by between 20% to 30% after the administering. In an aspect, the ALP level in the TBI patient undergoing hemorrhagic shock is reduced by between 20% to 40% after the administering. In an aspect, the ALP level in the TBI patient undergoing hemorrhagic shock is reduced by between 20% to 50% after the administering. In an aspect, the ALP level in the TBI patient undergoing hemorrhagic shock is reduced by between 30% to 40% after the administering. In an aspect, the ALP level in the TBI patient undergoing hemorrhagic shock is reduced by between 30% to 50% after the administering. In an aspect, the ALP level in the TBI patient undergoing hemorrhagic shock is reduced by between 40% to 50% after the administering. In an aspect, the ALP level in the TBI patient undergoing hemorrhagic shock is reduced by at least 10%, 20%, 30%, 40%, or 50% after the administering.

In an aspect of the present disclosure, the level of alkaline phosphatase (ALP) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the ALP level in the TBI patient undergoing hemorrhagic shock is reduced by between 1.5 and 3 fold after the administering. In an aspect, the ALP level in the TBI patient undergoing hemorrhagic shock is reduced by between 1.5 and 5 fold after the administering. In an aspect, the ALP level in the TBI patient undergoing hemorrhagic shock is reduced by between 1.5 and 10 fold after the administering. In an aspect, the ALP level in the TBI patient undergoing hemorrhagic shock is reduced by between 3 and 5 fold after the administering. In an aspect, the ALP level in the TBI patient undergoing hemorrhagic shock is reduced by between 3 and 10 fold after the administering. In an aspect, the ALP level in the TBI patient undergoing hemorrhagic shock is reduced by between 5 and 10 fold after the administering.

Methods of the present disclosure provide for, and include, reducing the level of alkaline phosphatase (ALP) in a TBI patient undergoing hemorrhagic shock, comprising administering to the patient oxygen reduced blood having an oxygen saturation (SO₂) of 20% or less prior to and during storage, compared to the level of ALP in a TBI patient undergoing hemorrhagic shock having been administered the same amount of non-oxygen reduced conventionally stored blood. In an aspect, the ALP level in the TBI patient undergoing hemorrhagic shock is reduced by at least 20% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by between 20% to 30% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by between 20% to 40% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by between 20% to 50% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by between 30% to 40% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by between 30% to 50% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by between 40% to 50% after administering, compared to a TBI patient undergoing hemorrhagic shock having been administered the same amount of non-oxygen reduced conventionally stored blood. In an aspect, the ALP level in the TBI patient undergoing hemorrhagic shock is reduced by at least 20%, 30%, 40%, or 50% after administering, compared to a TBI patient undergoing hemorrhagic shock having been administered the same amount of non-oxygen reduced conventionally stored blood.

Methods of the present disclosure provide for, and include, reducing the level of alkaline phosphatase (ALP) in a TBI patient undergoing hemorrhagic shock to between about 30 and about 200 international units per liter (IU/L), comprising administering to the patient oxygen reduced blood having an oxygen saturation (SO₂) of 20% or less prior to and during storage. In an aspect, the ALP level in the TBI patient undergoing hemorrhagic shock is reduced to between about 50 and about 200 IU/L after the administering. In an aspect, the ALP level in the TBI patient undergoing hemorrhagic shock is reduced to between about 100 and about 200 IU/L after the administering. In an aspect, the ALP level in the TBI patient undergoing hemorrhagic shock is reduced to between about 150 and about 200 IU/L after the administering. In an aspect, the ALP level in the TBI patient undergoing hemorrhagic shock is reduced to between about 50 and about 150 IU/L after the administering.

Methods of the present disclosure provide for, and include, reducing the level of aspartate aminotransferase (AST) in a TBI patient undergoing hemorrhagic shock, comprising administering to the patient oxygen reduced blood having an oxygen saturation (SO₂) of 20% or less prior to and during storage. In an aspect, the AST level in the TBI patient undergoing hemorrhagic shock is reduced by between 10% to 20% after the administering. In an aspect, the AST level in the TBI patient undergoing hemorrhagic shock is reduced by between 10% to 30% after the administering. In an aspect, the AST level in the TBI patient undergoing hemorrhagic shock is reduced by between 10% to 40% after the administering. In an aspect, the AST level in the TBI patient undergoing hemorrhagic shock is reduced by between 10% to 50% after the administering. In an aspect, the AST level in the TBI patient undergoing hemorrhagic shock is reduced by between 20% to 30% after the administering. In an aspect, the AST level in the TBI patient undergoing hemorrhagic shock is reduced by between 20% to 40% after the administering. In an aspect, the AST level in the TBI patient undergoing hemorrhagic shock is reduced by between 20% to 50% after the administering. In an aspect, the AST level in the TBI patient undergoing hemorrhagic shock is reduced by between 30% to 40% after the administering. In an aspect, the AST level in the TBI patient undergoing hemorrhagic shock is reduced by between 30% to 50% after the administering. In an aspect, the AST level in the TBI patient undergoing hemorrhagic shock is reduced by between 40% to 50% after the administering. In an aspect, the AST level in the TBI patient undergoing hemorrhagic shock is reduced by at least 10%, 20%, 30%, 40%, or 50% after the administering.

In an aspect of the present disclosure, the level of aspartate aminotransferase (AST) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the AST level in the TBI patient undergoing hemorrhagic shock is reduced by between 1.5 and 3 fold after the administering. In an aspect, the AST level in the TBI patient undergoing hemorrhagic shock is reduced by between 1.5 and 5 fold after the administering. In an aspect, the AST level in the TBI patient undergoing hemorrhagic shock is reduced by between 1.5 and 10 fold after the administering. In an aspect, the AST level in the TBI patient undergoing hemorrhagic shock is reduced by between 3 and 5 fold after the administering. In an aspect, the AST level in the TBI patient undergoing hemorrhagic shock is reduced by between 3 and 10 fold after the administering. In an aspect, the AST level in the TBI patient undergoing hemorrhagic shock is reduced by between 5 and 10 fold after the administering.

Methods of the present disclosure provide for, and include, reducing the level of aspartate aminotransferase (AST) in a TBI patient undergoing hemorrhagic shock, comprising administering to the patient oxygen reduced blood having an oxygen saturation (SO₂) of 20% or less prior to and during storage, compared to the level of AST in a TBI patient undergoing hemorrhagic shock having been administered the same amount of non-oxygen reduced conventionally stored blood. In an aspect, the AST level in the TBI patient undergoing hemorrhagic shock is reduced by at least 20% after administering, compared to a TBI patient undergoing hemorrhagic shock having been administered the same amount of non-oxygen reduced conventionally stored blood. In an aspect, the AST level in the TBI patient undergoing hemorrhagic shock is reduced by between 20% to 30% after administering, compared to a TBI patient undergoing hemorrhagic shock having been administered the same amount of non-oxygen reduced conventionally stored blood. In an aspect, the AST level in the TBI patient undergoing hemorrhagic shock is reduced by between 20% to 40% after administering, compared to a TBI patient undergoing hemorrhagic shock having been administered the same amount of non-oxygen reduced conventionally stored blood. In an aspect, the AST level in the TBI patient undergoing hemorrhagic shock is reduced by between 20% to 50% after administering, compared to a TBI patient undergoing hemorrhagic shock having been administered the same amount of non-oxygen reduced conventionally stored blood. In an aspect, the AST level in the TBI patient undergoing hemorrhagic shock is reduced by between 30% to 40% after administering, compared to a TBI patient undergoing hemorrhagic shock having been administered the same amount of non-oxygen reduced conventionally stored blood. In an aspect, the AST level in the TBI patient undergoing hemorrhagic shock is reduced by between 30% to 50% after administering, compared to a TBI patient undergoing hemorrhagic shock having been administered the same amount of non-oxygen reduced conventionally stored blood. In an aspect, the AST level in the TBI patient undergoing hemorrhagic shock is reduced by between 40% to 50% after administering, compared to a TBI patient undergoing hemorrhagic shock having been administered the same amount of non-oxygen reduced conventionally stored blood. In an aspect, the AST level in the TBI patient undergoing hemorrhagic shock is reduced by at least 20%, 30%, 40%, or 50% after administering, compared to a TBI patient undergoing hemorrhagic shock having been administered the same amount of non-oxygen reduced conventionally stored blood.

Methods of the present disclosure provide for, and include, reducing the level of aspartate aminotransferase (AST) in a TBI patient undergoing hemorrhagic shock to between about 5 and about 50 units per liter (U/L), comprising administering to the patient oxygen reduced blood having an oxygen saturation (SO₂) of 20% or less prior to and during storage. In an aspect, the AST level in the TBI patient undergoing hemorrhagic shock is reduced to between about 10 and about 50 U/L after the administering. In an aspect, the AST level in the TBI patient undergoing hemorrhagic shock is reduced to between about 25 and about 50 U/L after the administering. In an aspect, the AST level in the TBI patient undergoing hemorrhagic shock is reduced to between about 5 and about 40 U/L after the administering. In an aspect, the AST level in the TBI patient undergoing hemorrhagic shock is reduced to between about 30 and about 50 U/L after the administering.

Methods of the present disclosure provide for, and include, reducing the level of albumin in a TBI patient undergoing hemorrhagic shock, comprising administering to the patient oxygen reduced blood having an oxygen saturation (SO₂) of 20% or less prior to and during storage. In an aspect, the albumin level in the TBI patient undergoing hemorrhagic shock is reduced by between 10% to 20% after the administering. In an aspect, the albumin level in the TBI patient undergoing hemorrhagic shock is reduced by between 10% to 30% after the administering. In an aspect, the albumin level in the TBI patient undergoing hemorrhagic shock is reduced by between 10% to 40% after the administering. In an aspect, the albumin level in the TBI patient undergoing hemorrhagic shock is reduced by between 10% to 50% after the administering. In an aspect, the albumin level in the TBI patient undergoing hemorrhagic shock is reduced by between 20% to 30% after the administering. In an aspect, the albumin level in the TBI patient undergoing hemorrhagic shock is reduced by between 20% to 40% after the administering. In an aspect, the albumin level in the TBI patient undergoing hemorrhagic shock is reduced by between 20% to 50% after the administering. In an aspect, the albumin level in the TBI patient undergoing hemorrhagic shock is reduced by between 30% to 40% after the administering. In an aspect, the albumin level in the TBI patient undergoing hemorrhagic shock is reduced by between 30% to 50% after the administering. In an aspect, the albumin level in the TBI patient undergoing hemorrhagic shock is reduced by between 40% to 50% after the administering. In an aspect, the albumin level in the TBI patient undergoing hemorrhagic shock is reduced by at least 10%, 20%, 30%, 40%, or 50% after the administering.

In an aspect of the present disclosure, the level of albumin in a TBI patient undergoing hemorrhagic shock is reduced by between 1.5 and 10 fold after administering oxygen reduced blood having an oxygen saturation (SO₂) of 20% or less prior to and during storage to the patient. In an aspect, the albumin level in the TBI patient undergoing hemorrhagic shock is reduced by between 1.5 and 3 fold after the administering. In an aspect, the albumin level in the TBI patient undergoing hemorrhagic shock is reduced by between 1.5 and 5 fold after the administering. In an aspect, the albumin level in the TBI patient undergoing hemorrhagic shock is reduced by between 1.5 and 10 fold after the administering. In an aspect, the albumin level in the TBI patient undergoing hemorrhagic shock is reduced by between 3 and 5 fold after the administering. In an aspect, the albumin level in the TBI patient undergoing hemorrhagic shock is reduced by between 3 and 10 fold after the administering. In an aspect, the albumin level in the TBI patient undergoing hemorrhagic shock is reduced by between 5 and 10 fold after the administering.

Methods of the present disclosure provide for, and include, reducing the level of albumin in a TBI patient undergoing hemorrhagic shock, comprising administering to the patient oxygen reduced blood having an oxygen saturation (SO₂) of 20% or less prior to and during storage, compared to the level of albumin in a TBI patient undergoing hemorrhagic shock having been administered the same amount of non-oxygen reduced conventionally stored blood. In an aspect, the albumin level in the TBI patient undergoing hemorrhagic shock is reduced by at least 20% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by between 20% to 50% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the albumin level in the TBI patient undergoing hemorrhagic shock is reduced by at least 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of albumin in a TBI patient undergoing hemorrhagic shock to between about 1.5 and about 8 grams per deciliter (g/dL), comprising administering to the patient oxygen reduced blood having an oxygen saturation (SO₂) of 20% or less prior to and during storage. In an aspect, the albumin level in the TBI patient undergoing hemorrhagic shock is reduced to between about 3 and about 8 g/dL after the administering. In an aspect, the albumin level in the TBI patient undergoing hemorrhagic shock is reduced to between about 5 and about 8 g/dL after the administering. In an aspect, the albumin level in the TBI patient undergoing hemorrhagic shock is reduced to between about 3 and about 6 g/dL after the administering.

Methods of the present disclosure provide for, and include, reducing the level of albumin plus globulin (total protein) in a TBI patient undergoing hemorrhagic shock, comprising administering to patient oxygen reduced blood having an oxygen saturation (SO₂) of 20% or less prior to and during storage. In an aspect, the total protein level in the TBI patient undergoing hemorrhagic shock is reduced by between 10% to 20% after the administering. In an aspect, the total protein level in the TBI patient undergoing hemorrhagic shock is reduced by between 10% to 30% after the administering. In an aspect, the total protein level in the TBI patient undergoing hemorrhagic shock is reduced by between 10% to 40% after the administering. In an aspect, the total protein level in the TBI patient undergoing hemorrhagic shock is reduced by between 10% to 50% after the administering. In an aspect, the total protein level in the TBI patient undergoing hemorrhagic shock is reduced by between 20% to 30% after the administering. In an aspect, the total protein level in the TBI patient undergoing hemorrhagic shock is reduced by between 20% to 40% after the administering. In an aspect, the total protein level in the TBI patient undergoing hemorrhagic shock is reduced by between 20% to 50% after the administering. In an aspect, the total protein level in the TBI patient undergoing hemorrhagic shock is reduced by between 30% to 40% after the administering. In an aspect, the total protein level in the TBI patient undergoing hemorrhagic shock is reduced by between 30% to 50% after the administering. In an aspect, the total protein level in the TBI patient undergoing hemorrhagic shock is reduced by between 40% to 50% after the administering. In an aspect, the total protein level in the TBI patient undergoing hemorrhagic shock is reduced by at least 10%, 20%, 30%, 40%, or 50% after the administering.

In an aspect of the present disclosure, the level of albumin plus globulin (total protein) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the total protein level in the TBI patient undergoing hemorrhagic shock is reduced by between 1.5 and 3 fold after the administering. In an aspect, the total protein level in the TBI patient undergoing hemorrhagic shock is reduced by between 1.5 and 5 fold after the administering. In an aspect, the total protein level in the TBI patient undergoing hemorrhagic shock is reduced by between 1.5 and 10 fold after the administering. In an aspect, the total protein level in the TBI patient undergoing hemorrhagic shock is reduced by between 3 and 5 fold after the administering. In an aspect, the total protein level in the TBI patient undergoing hemorrhagic shock is reduced by between 3 and 10 fold after the administering. In an aspect, the total protein level in the TBI patient undergoing hemorrhagic shock is reduced by between 5 and 10 fold after the administering.

Methods of the present disclosure provide for, and include, reducing the level of albumin plus globulin (total protein) in a TBI patient undergoing hemorrhagic shock, comprising administering to the patient oxygen reduced blood having an oxygen saturation (SO₂) of 20% or less prior to and during storage, compared to the level of total protein in a TBI patient undergoing hemorrhagic shock having been administered the same amount of non-oxygen reduced conventionally stored blood. In an aspect, the total protein level in the TBI patient undergoing hemorrhagic shock is reduced by at least 20% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by between 20% to 50% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the total protein level in the TBI patient undergoing hemorrhagic shock is reduced by at least 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of total protein plus globulin (total protein) in a TBI patient undergoing hemorrhagic shock to between about 2 and about 12 grams per deciliter (g/dL), comprising administering to the patient oxygen reduced blood having an oxygen saturation (SO₂) of 20% or less prior to and during storage. In an aspect, the total protein level in the TBI patient undergoing hemorrhagic shock is reduced to between about 8 and about 12 g/dL after the administering.

In an aspect, the total protein level in the TBI patient undergoing hemorrhagic shock is reduced to between about 5 and about 12 g/dL after the administering. In an aspect, the total protein level in the TBI patient undergoing hemorrhagic shock is reduced to between about 2 and about 10 g/dL after the administering. In an aspect, the total protein level in the TBI patient undergoing hemorrhagic shock is reduced to between about 5 and about 10 g/dL after the administering.

Methods of the present disclosure provide for, and include, reducing the level of bilirubin in a TBI patient undergoing hemorrhagic shock, comprising administering to the patient oxygen reduced blood having an oxygen saturation (SO₂) of 20% or less prior to and during storage. In an aspect, the bilirubin level in the TBI patient undergoing hemorrhagic shock is reduced by between 10% to 20% after the administering. In an aspect, the bilirubin level in the TBI patient undergoing hemorrhagic shock is reduced by between 10% to 30% after the administering. In an aspect, the bilirubin level in the TBI patient undergoing hemorrhagic shock is reduced by between 10% to 40% after the administering. In an aspect, the bilirubin level in the TBI patient undergoing hemorrhagic shock is reduced by between 10% to 50% after the administering. In an aspect, the bilirubin level in the TBI patient undergoing hemorrhagic shock is reduced by between 20% to 30% after the administering. In an aspect, the bilirubin level in the TBI patient undergoing hemorrhagic shock is reduced by between 20% to 40% after the administering. In an aspect, the bilirubin level in the TBI patient undergoing hemorrhagic shock is reduced by between 20% to 50% after the administering. In an aspect, the bilirubin level in the TBI patient undergoing hemorrhagic shock is reduced by between 30% to 40% after the administering. In an aspect, the bilirubin level in the TBI patient undergoing hemorrhagic shock is reduced by between 30% to 50% after the administering. In an aspect, the bilirubin level in the TBI patient undergoing hemorrhagic shock is reduced by between 40% to 50% after the administering. In an aspect, the bilirubin level in the TBI patient undergoing hemorrhagic shock is reduced by at least 10%, 20%, 30%, 40%, or 50% after the administering.

In an aspect of the present disclosure, the level of bilirubin in a TBI patient undergoing hemorrhagic shock is reduced by between 1.5 and 10 fold after administering oxygen reduced blood having an oxygen saturation (SO₂) of 20% or less prior to and during storage to the patient. In an aspect, the bilirubin level in the TBI patient undergoing hemorrhagic shock is reduced by between 1.5 and 3 fold after the administering. In an aspect, the bilirubin level in the TBI patient undergoing hemorrhagic shock is reduced by between 1.5 and 5 fold after the administering. In an aspect, the bilirubin level in the TBI patient undergoing hemorrhagic shock is reduced by between 1.5 and 10 fold after the administering. In an aspect, the bilirubin level in the TBI patient undergoing hemorrhagic shock is reduced by between 3 and 5 fold after the administering. In an aspect, the bilirubin level in the TBI patient undergoing hemorrhagic shock is reduced by between 3 and 10 fold after the administering. In an aspect, the bilirubin level in the TBI patient undergoing hemorrhagic shock is reduced by between 5 and 10 fold after the administering.

Methods of the present disclosure provide for, and include, reducing the level of bilirubin in a TBI patient undergoing hemorrhagic shock, comprising administering to the patient oxygen reduced blood having an oxygen saturation (SO₂) of 20% or less prior to and during storage, compared to the level of bilirubin in a TBI patient undergoing hemorrhagic shock having been administered the same amount of non-oxygen reduced conventionally stored blood. In an aspect, the bilirubin level in the TBI patient undergoing hemorrhagic shock is reduced by at least 20% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by between 20% to 50% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the bilirubin level in the TBI patient undergoing hemorrhagic shock is reduced by at least 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of bilirubin in a TBI patient undergoing hemorrhagic shock to between about 5 and about 120 millimoles per liter (mmol/L), comprising administering to the patient oxygen reduced blood having an oxygen saturation (SO₂) of 20% or less prior to and during storage. In an aspect, the bilirubin level in the TBI patient undergoing hemorrhagic shock is reduced to between about 30 and about 120 mmol/L after the administering. In an aspect, the bilirubin level in the TBI patient undergoing hemorrhagic shock is reduced to between about 50 and about 120 mmol/L after the administering. In an aspect, the bilirubin level in the TBI patient undergoing hemorrhagic shock is reduced to between about 80 and about 120 mmol/L after the administering. In an aspect, the bilirubin level in the TBI patient undergoing hemorrhagic shock is reduced to between about 5 and about 50 mmol/L after the administering. In an aspect, the bilirubin level in the TBI patient undergoing hemorrhagic shock is reduced to between about 30 and about 100 mmol/L after the administering. In an aspect, the bilirubin level in the TBI patient undergoing hemorrhagic shock is reduced to between about 50 and about 100 mmol/L after the administering.

Methods of the present disclosure provide for, and include, reducing the level of interleukin-6 (IL-6) in a TBI patient undergoing hemorrhagic shock, comprising administering to the patient oxygen reduced blood having an oxygen saturation (SO₂) of 20% or less prior to and during storage. In an aspect, the IL-6 level in the TBI patient undergoing hemorrhagic shock is reduced by between 10% to 20% after the administering. In an aspect, the IL-6 level in the TBI patient undergoing hemorrhagic shock is reduced by between 10% to 30% after the administering. In an aspect, the IL-6 level in the TBI patient undergoing hemorrhagic shock is reduced by between 10% to 40% after the administering. In an aspect, the IL-6 level in the TBI patient undergoing hemorrhagic shock is reduced by between 10% to 50% after the administering. In an aspect, the IL-6 level in the TBI patient undergoing hemorrhagic shock is reduced by between 20% to 30% after the administering. In an aspect, the IL-6 level in the TBI patient undergoing hemorrhagic shock is reduced by between 20% to 40% after the administering. In an aspect, the IL-6 level in the TBI patient undergoing hemorrhagic shock is reduced by between 20% to 50% after the administering. In an aspect, the IL-6 level in the TBI patient undergoing hemorrhagic shock is reduced by between 30% to 40% after the administering. In an aspect, the IL-6 level in the TBI patient undergoing hemorrhagic shock is reduced by between 30% to 50% after the administering. In an aspect, the IL-6 level in the TBI patient undergoing hemorrhagic shock is reduced by between 40% to 50% after the administering. In an aspect, the IL-6 level in the TBI patient undergoing hemorrhagic shock is reduced by at least 10%, 20%, 30%, 40%, or 50% after the administering.

In an aspect of the present disclosure, the level of interleukin-6 (IL-6) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the IL-6 level in the TBI patient undergoing hemorrhagic shock is reduced by between 1.5 and 3 fold after the administering. In an aspect, the IL-6 level in the TBI patient undergoing hemorrhagic shock is reduced by between 1.5 and 5 fold after the administering. In an aspect, the IL-6 level in the TBI patient undergoing hemorrhagic shock is reduced by between 1.5 and 10 fold after the administering. In an aspect, the IL-6 level in the TBI patient undergoing hemorrhagic shock is reduced by between 3 and 5 fold after the administering. In an aspect, the IL-6 level in the TBI patient undergoing hemorrhagic shock is reduced by between 3 and 10 fold after the administering. In an aspect, the IL-6 level in the TBI patient undergoing hemorrhagic shock is reduced by between 5 and 10 fold after the administering.

Methods of the present disclosure provide for, and include, reducing the level of interleukin-6 (IL-6) in a TBI patient undergoing hemorrhagic shock, comprising administering to the patient oxygen reduced blood having an oxygen saturation (SO₂) of 20% or less prior to and during storage, compared to a TBI patient undergoing hemorrhagic shock having been administered the same amount of non-oxygen reduced conventionally stored blood. In an aspect, the IL-6 level in the TBI patient undergoing hemorrhagic shock is reduced by at least 20% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by between 20% to 50% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the IL-6 level in the TBI patient undergoing hemorrhagic shock is reduced by at least 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of interleukin-6 (IL-6) in a TBI patient undergoing hemorrhagic shock to between about 3 and about 8 picograms per milliliter (pg/mL), comprising administering to the patient oxygen reduced blood having an oxygen saturation (SO₂) of 20% or less prior to and during storage. In an aspect, the IL-6 level in the TBI patient undergoing hemorrhagic shock is reduced to between about 4 and about 8 pg/mL after the administering. In an aspect, the IL-6 level in the TBI patient undergoing hemorrhagic shock is reduced to between about 6 and about 8 pg/mL after the administering. In an aspect, the IL-6 level in the TBI patient undergoing hemorrhagic shock is reduced to between about 3 and about 6 pg/mL after the administering. In an aspect, the IL-6 level in the TBI patient undergoing hemorrhagic shock is reduced to between about 3 and about 4 pg/mL after the administering.

Methods of the present disclosure provide for, and include, reducing the level of tumor necrosis factor alpha (TNF-α) in a TBI patient undergoing hemorrhagic shock, comprising administering to the patient oxygen reduced blood having an oxygen saturation (SO₂) of 20% or less prior to and during storage. In an aspect, the TNF-α level in the TBI patient undergoing hemorrhagic shock is reduced by between 10% to 20% after the administering. In an aspect, the TNF-α level in the TBI patient undergoing hemorrhagic shock is reduced by between 10% to 30% after the administering. In an aspect, the TNF-α level in the TBI patient undergoing hemorrhagic shock is reduced by between 10% to 40% after the administering. In an aspect, the TNF-α level in the TBI patient undergoing hemorrhagic shock is reduced by between 10% to 50% after the administering. In an aspect, the TNF-α level in the TBI patient undergoing hemorrhagic shock is reduced by between 20% to 30% after the administering. In an aspect, the TNF-α level in the TBI patient undergoing hemorrhagic shock is reduced by between 20% to 40% after the administering. In an aspect, the TNF-α level in the TBI patient undergoing hemorrhagic shock is reduced by between 20% to 50% after the administering. In an aspect, the TNF-α level in the TBI patient undergoing hemorrhagic shock is reduced by between 30% to 40% after the administering. In an aspect, the TNF-α level in the TBI patient undergoing hemorrhagic shock is reduced by between 30% to 50% after the administering. In an aspect, the TNF-α level in the TBI patient undergoing hemorrhagic shock is reduced by between 40% to 50% after the administering. In an aspect, the TNF-α level in the TBI patient undergoing hemorrhagic shock is reduced by at least 10%, 20%, 30%, 40%, or 50% after the administering.

In an aspect of the present disclosure, the level of tumor necrosis factor alpha (TNF-α) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the TNF-α level in the TBI patient undergoing hemorrhagic shock is reduced by between 1.5 and 3 fold after the administering. In an aspect, the TNF-α level in the TBI patient undergoing hemorrhagic shock is reduced by between 1.5 and 5 fold after the administering. In an aspect, the TNF-α level in the TBI patient undergoing hemorrhagic shock is reduced by between 1.5 and 10 fold after the administering. In an aspect, the TNF-α level in the TBI patient undergoing hemorrhagic shock is reduced by between 3 and 5 fold after the administering. In an aspect, the TNF-α level in the TBI patient undergoing hemorrhagic shock is reduced by between 3 and 10 fold after the administering. In an aspect, the TNF-α level in the TBI patient undergoing hemorrhagic shock is reduced by between 5 and 10 fold after the administering.

Methods of the present disclosure provide for, and include, reducing the level of tumor necrosis factor alpha (TNF-α) in a TBI patient undergoing hemorrhagic shock, comprising administering to the patient oxygen reduced blood having an oxygen saturation (SO₂) of 20% or less prior to and during storage, compared to a TBI patient undergoing hemorrhagic shock having been administered the same amount of non-oxygen reduced conventionally stored blood. In an aspect, the TNF-α level in the TBI patient undergoing hemorrhagic shock is reduced by at least 20% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by between 20% to 50% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the TNF-α level in the TBI patient undergoing hemorrhagic shock is reduced by at least 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of tumor necrosis factor alpha (TNF-α) in a TBI patient undergoing hemorrhagic shock to between about 50 and about 100 picograms per milliliter (pg/mL), comprising administering to the patient oxygen reduced blood having an oxygen saturation (SO₂) of 20% or less prior to and during storage. In an aspect, the TNF-α level in the TBI patient undergoing hemorrhagic shock is reduced to between about 50 and about 70 pg/mL after the administering. In an aspect, the TNF-α level in the TBI patient undergoing hemorrhagic shock is reduced to between about 50 and about 90 pg/mL after the administering. In an aspect, the TNF-α level in the TBI patient undergoing hemorrhagic shock is reduced to between about 70 and about 90 pg/mL after the administering. In an aspect, the TNF-α level in the TBI patient undergoing hemorrhagic shock is reduced to between about 70 and about 100 pg/mL after the administering.

Methods of the present disclosure provide for, and include, reducing the level of monocyte chemoattractant protein-1 (MCP1) in a TBI patient undergoing hemorrhagic shock, comprising administering to the patient oxygen reduced blood having an oxygen saturation (SO₂) of 20% or less prior to and during storage. In an aspect, the MCP1 level in the TBI patient undergoing hemorrhagic shock is reduced by between 10% to 20% after the administering. In an aspect, the MCP1 level in the TBI patient undergoing hemorrhagic shock is reduced by between 10% to 30% after the administering. In an aspect, the MCP1 level in the TBI patient undergoing hemorrhagic shock is reduced by between 10% to 40% after the administering. In an aspect, the MCP1 level in the TBI patient undergoing hemorrhagic shock is reduced by between 10% to 50% after the administering. In an aspect, the MCP1 level in the TBI patient undergoing hemorrhagic shock is reduced by between 20% to 30% after the administering. In an aspect, the MCP1 level in the TBI patient undergoing hemorrhagic shock is reduced by between 20% to 40% after the administering. In an aspect, the MCP1 level in the TBI patient undergoing hemorrhagic shock is reduced by between 20% to 50% after the administering. In an aspect, the MCP1 level in the TBI patient undergoing hemorrhagic shock is reduced by between 30% to 40% after the administering. In an aspect, the MCP1 level in the TBI patient undergoing hemorrhagic shock is reduced by between 30% to 50% after the administering. In an aspect, the MCP1 level in the TBI patient undergoing hemorrhagic shock is reduced by between 40% to 50% after the administering. In an aspect, the MCP1 level in the TBI patient undergoing hemorrhagic shock is reduced by at least 10%, 20%, 30%, 40%, or 50% after the administering.

In an aspect of the present disclosure, the level of monocyte chemoattractant protein-1 (MCP1) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the MCP1 level in the TBI patient undergoing hemorrhagic shock is reduced by between 1.5 and 3 fold after the administering. In an aspect, the MCP1 level in the TBI patient undergoing hemorrhagic shock is reduced by between 1.5 and 5 fold after the administering. In an aspect, the MCP1 level in the TBI patient undergoing hemorrhagic shock is reduced by between 1.5 and 10 fold after the administering. In an aspect, the MCP1 level in the TBI patient undergoing hemorrhagic shock is reduced by between 3 and 5 fold after the administering. In an aspect, the MCP1 level in the TBI patient undergoing hemorrhagic shock is reduced by between 3 and 10 fold after the administering. In an aspect, the MCP1 level in the TBI patient undergoing hemorrhagic shock is reduced by between 5 and 10 fold after the administering.

Methods of the present disclosure provide for, and include, reducing the level of monocyte chemoattractant protein-1 MCP1 in a TBI patient undergoing hemorrhagic shock, comprising administering to the patient oxygen reduced blood having an oxygen saturation (SO₂) of 20% or less prior to and during storage, compared to a TBI patient undergoing hemorrhagic shock having been administered the same amount of non-oxygen reduced conventionally stored blood. In an aspect, the MCP1 level in the TBI patient undergoing hemorrhagic shock is reduced by at least 20% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by between 20% to 50% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by between 30% to 40% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by between 30% to 50% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by between 40% to 50% after the administering, compared to a TBI patient undergoing hemorrhagic shock having been administered the same amount of non-oxygen reduced conventionally stored blood. In an aspect, the MCP1 level in the TBI patient undergoing hemorrhagic shock is reduced by at least 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of monocyte chemoattractant protein-1 (MCP1) in a TBI patient undergoing hemorrhagic shock to between about 50 and about 200 picograms per milliliter (pg/mL), comprising administering to the patient oxygen reduced blood having an oxygen saturation (SO₂) of 20% or less prior to and during storage. In an aspect, the MCP1 level in the TBI patient undergoing hemorrhagic shock is reduced to between about 75 and about 200 pg/mL after the administering. In an aspect, the MCP1 level in the TBI patient undergoing hemorrhagic shock is reduced to between about 100 and about 200 pg/mL after the administering. In an aspect, the MCP1 level in the TBI patient undergoing hemorrhagic shock is reduced to between about 150 and about 200 pg/mL after the administering. In an aspect, the MCP1 level in the TBI patient undergoing hemorrhagic shock is reduced to between about 50 and about 150 pg/mL after the administering. In an aspect, the MCP1 level in the TBI patient undergoing hemorrhagic shock is reduced to between about 50 and about 100 pg/mL after the administering. In an aspect, the MCP1 level in the TBI patient undergoing hemorrhagic shock is reduced to between about 50 and about 75 pg/mL after the administering.

Methods of the present disclosure provide for, and include, reducing the level of ferritin in a TBI patient undergoing hemorrhagic shock, comprising administering to the patient oxygen reduced blood having an oxygen saturation (SO₂) of 20% or less prior to and during storage. In an aspect, the ferritin level in the TBI patient undergoing hemorrhagic shock is reduced by between 10% to 20% after the administering. In an aspect, the ferritin level in the TBI patient undergoing hemorrhagic shock is reduced by between 10% to 30% after the administering. In an aspect, the ferritin level in the TBI patient undergoing hemorrhagic shock is reduced by between 10% to 40% after the administering. In an aspect, the ferritin level in the TBI patient undergoing hemorrhagic shock is reduced by between 10% to 50% after the administering. In an aspect, the ferritin level in the TBI patient undergoing hemorrhagic shock is reduced by between 20% to 30% after the administering. In an aspect, the ferritin level in the TBI patient undergoing hemorrhagic shock is reduced by between 20% to 40% after the administering. In an aspect, the ferritin level in the TBI patient undergoing hemorrhagic shock is reduced by between 20% to 50% after the administering. In an aspect, the ferritin level in the TBI patient undergoing hemorrhagic shock is reduced by between 30% to 40% after the administering. In an aspect, the ferritin level in the TBI patient undergoing hemorrhagic shock is reduced by between 30% to 50% after the administering. In an aspect, the ferritin level in the TBI patient undergoing hemorrhagic shock is reduced by between 40% to 50% after the administering. In an aspect, the ferritin level in the TBI patient undergoing hemorrhagic shock is reduced by at least 10%, 20%, 30%, 40%, or 50% after the administering.

In an aspect of the present disclosure, the level of ferritin in a TBI patient undergoing hemorrhagic shock is reduced by between 1.5 and 10 fold after administering oxygen reduced blood having an oxygen saturation (SO₂) of 20% or less prior to and during storage to the patient. In an aspect, the ferritin level in the TBI patient undergoing hemorrhagic shock is reduced by between 1.5 and 3 fold after the administering. In an aspect, the ferritin level in the TBI patient undergoing hemorrhagic shock is reduced by between 1.5 and 5 fold after the administering. In an aspect, the ferritin level in the TBI patient undergoing hemorrhagic shock is reduced by between 1.5 and 10 fold after the administering. In an aspect, the ferritin level in the TBI patient undergoing hemorrhagic shock is reduced by between 3 and 5 fold after the administering. In an aspect, the ferritin level in the TBI patient undergoing hemorrhagic shock is reduced by between 3 and 10 fold after the administering. In an aspect, the ferritin level in the TBI patient undergoing hemorrhagic shock is reduced by between 5 and 10 fold after the administering.

Methods of the present disclosure provide for, and include, reducing the level of ferritin in a TBI patient undergoing hemorrhagic shock, comprising administering to the patient oxygen reduced blood having an oxygen saturation (SO₂) of 20% or less prior to and during storage, compared to the level of ferritin in a TBI patient undergoing hemorrhagic shock having been administered the same amount of non-oxygen reduced conventionally stored blood. In an aspect, the ferritin level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the ferritin level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the ferritin level in the TBI patient undergoing hemorrhagic shock is reduced by between 5% to 15% after the administering, compared to a TBI patient undergoing hemorrhagic shock having been administered the same amount of non-oxygen reduced conventionally stored blood. In an aspect, the ferritin level in the TBI patient undergoing hemorrhagic shock is reduced by between 10% to 15% after the administering, compared to a TBI patient undergoing hemorrhagic shock having been administered the same amount of non-oxygen reduced conventionally stored blood. In an aspect, the ferritin level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the ferritin level in the TBI patient undergoing hemorrhagic shock is reduced by between 15% to 20% after the administering, compared to a TBI patient undergoing hemorrhagic shock having been administered the same amount of non-oxygen reduced conventionally stored blood. In an aspect, the ferritin level in the TBI patient undergoing hemorrhagic shock is reduced by at least 5%, 10%, 15%, or 20% after the administering, compared to a TBI patient undergoing hemorrhagic shock having been administered the same amount of non-oxygen reduced conventionally stored blood.

Methods of the present disclosure provide for, and include, reducing the level of ferritin in a TBI patient undergoing hemorrhagic shock to between about 5 and about 260 nanograms per milliliter (ng/mL), comprising administering to the patient oxygen reduced blood having an oxygen saturation (SO₂) of 20% or less prior to and during storage. In an aspect, the ferritin level in the TBI patient undergoing hemorrhagic shock is reduced to between about 50 and about 260 ng/mL after the administering. In an aspect, the ferritin level in the TBI patient undergoing hemorrhagic shock is reduced to between about 100 and about 260 ng/mL after the administering. In an aspect, the ferritin level in the TBI patient undergoing hemorrhagic shock is reduced to between about 150 and about 260 ng/ml after the administering. In an aspect, the ferritin level in the TBI patient undergoing hemorrhagic shock is reduced to between about 200 and about 260 ng/mL after the administering.

Methods of the present disclosure provide for, and include, reducing the level of troponin in a TBI patient undergoing hemorrhagic shock, comprising administering to the patient oxygen reduced blood having an oxygen saturation (SO₂) of 20% or less prior to and during storage. In an aspect, the troponin level in the TBI patient undergoing hemorrhagic shock is reduced by between 10% to 20% after the administering. In an aspect, the troponin level in the TBI patient undergoing hemorrhagic shock is reduced by between 10% to 30% after the administering. In an aspect, the troponin level in the TBI patient undergoing hemorrhagic shock is reduced by between 10% to 40% after the administering. In an aspect, the troponin level in the TBI patient undergoing hemorrhagic shock is reduced by between 10% to 50% after the administering. In an aspect, the troponin level in the TBI patient undergoing hemorrhagic shock is reduced by between 20% to 30% after the administering. In an aspect, the troponin level in the TBI patient undergoing hemorrhagic shock is reduced by between 20% to 40% after the administering. In an aspect, the troponin level in the TBI patient undergoing hemorrhagic shock is reduced by between 20% to 50% after the administering. In an aspect, the troponin level in the TBI patient undergoing hemorrhagic shock is reduced by between 30% to 40% after the administering. In an aspect, the troponin level in the TBI patient undergoing hemorrhagic shock is reduced by between 30% to 50% after the administering. In an aspect, the troponin level in the TBI patient undergoing hemorrhagic shock is reduced by between 40% to 50% after the administering. In an aspect, the troponin level in the TBI patient undergoing hemorrhagic shock is reduced by at least 10%, 20%, 30%, 40%, or 50% after the administering.

In an aspect of the present disclosure, the level of troponin in the TBI patient undergoing hemorrhagic shock is reduced by between 1.5 and 10 fold after administering oxygen reduced blood having an oxygen saturation (SO₂) of 20% or less prior to and during storage to the patient. In an aspect, the troponin level in the TBI patient undergoing hemorrhagic shock is reduced by between 1.5 and 3 fold after the administering. In an aspect, the troponin level in the TBI patient undergoing hemorrhagic shock is reduced by between 1.5 and 5 fold after the administering. In an aspect, the troponin level in the TBI patient undergoing hemorrhagic shock is reduced by between 1.5 and 10 fold after the administering. In an aspect, the troponin level in the TBI patient undergoing hemorrhagic shock is reduced by between 3 and 5 fold after the administering. In an aspect, the troponin level in the TBI patient undergoing hemorrhagic shock is reduced by between 3 and 10 fold after the administering. In an aspect, the troponin level in the TBI patient undergoing hemorrhagic shock is reduced by between 5 and 10 fold after the administering.

Methods of the present disclosure provide for, and include, reducing the level of troponin in a TBI patient undergoing hemorrhagic shock, comprising administering to the patient oxygen reduced blood having an oxygen saturation (SO₂) of 20% or less prior to and during storage, compared to the level of troponin in a TBI patient undergoing hemorrhagic shock having been administered the same amount of non-oxygen reduced conventionally stored blood. In an aspect, the troponin level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the troponin level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of troponin in a TBI patient undergoing hemorrhagic shock to between about 0.01 and about 0.1 nanograms per milliliter (ng/mL), comprising administering to the patient oxygen reduced blood having an oxygen saturation (SO₂) of 20% or less prior to and during storage. In an aspect, the troponin level in the TBI patient undergoing hemorrhagic shock is reduced to between about 0.02 and about 0.1 ng/mL after the administering. In an aspect, the troponin level in the TBI patient undergoing hemorrhagic shock is reduced to between about 0.04 and about 0.1 ng/mL after the administering. In an aspect, the troponin level in the TBI patient undergoing hemorrhagic shock is reduced to between about 0.06 and about 0.1 ng/ml after the administering. In an aspect, the troponin level in the TBI patient undergoing hemorrhagic shock is reduced to between about 0.08 and about 0.1 ng/ml after the administering.

Methods of the present disclosure provide for, and include, reducing the level of plasma C reactive protein (CRP) in a TBI patient undergoing hemorrhagic shock, comprising administering to the patient oxygen reduced blood having an oxygen saturation (SO₂) of 20% or less prior to and during storage. In an aspect, the CRP level in the TBI patient undergoing hemorrhagic shock is reduced by between 10% to 20% after the administering. In an aspect, the CRP level in the TBI patient undergoing hemorrhagic shock is reduced by between 10% to 30% after the administering. In an aspect, the CRP level in the TBI patient undergoing hemorrhagic shock is reduced by between 10% to 40% after the administering. In an aspect, the CRP level in the TBI patient undergoing hemorrhagic shock is reduced by between 10% to 50% after the administering. In an aspect, the CRP level in the TBI patient undergoing hemorrhagic shock is reduced by between 20% to 30% after the administering. In an aspect, the CRP level in the TBI patient undergoing hemorrhagic shock is reduced by between 20% to 40% after the administering. In an aspect, the CRP level in the TBI patient undergoing hemorrhagic shock is reduced by between 20% to 50% after the administering. In an aspect, the CRP level in the TBI patient undergoing hemorrhagic shock is reduced by between 30% to 40% after the administering. In an aspect, the CRP level in the TBI patient undergoing hemorrhagic shock is reduced by between 30% to 50% after the administering. In an aspect, the CRP level in the TBI patient undergoing hemorrhagic shock is reduced by between 40% to 50% after the administering. In an aspect, the CRP level in the TBI patient undergoing hemorrhagic shock is reduced by at least 10%, 20%, 30%, 40%, or 50% after the administering.

In an aspect of the present disclosure, the level of plasma C reactive protein (CRP) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the CRP level in the TBI patient undergoing hemorrhagic shock is reduced by between 1.5 and 3 fold after the administering. In an aspect, the CRP level in the TBI patient undergoing hemorrhagic shock is reduced by between 1.5 and 5 fold after the administering. In an aspect, the CRP level in the TBI patient undergoing hemorrhagic shock is reduced by between 1.5 and 10 fold after the administering. In an aspect, the CRP level in the TBI patient undergoing hemorrhagic shock is reduced by between 3 and 5 fold after the administering. In an aspect, the CRP level in the TBI patient undergoing hemorrhagic shock is reduced by between 3 and 10 fold after the administering. In an aspect, the CRP level in the TBI patient undergoing hemorrhagic shock is reduced by between 5 and 10 fold after the administering.

Methods of the present disclosure provide for, and include, reducing the level of plasma C reactive protein (CRP) in a TBI patient undergoing hemorrhagic shock, comprising administering to the patient oxygen reduced blood having an oxygen saturation (SO₂) of 20% or less prior to and during storage, compared to the level of CRP in a TBI patient undergoing hemorrhagic shock having been administered the same amount of non-oxygen reduced conventionally stored blood. In an aspect, the CRP level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the CRP level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of plasma C reactive protein (CRP) in a TBI patient undergoing hemorrhagic shock to between about 5 and about 15 milligrams per milliliter (mg/mL), comprising administering to the patient oxygen reduced blood having an oxygen saturation (SO₂) of 20% or less prior to and during storage. In an aspect, the ferritin level in the TBI patient undergoing hemorrhagic shock is reduced to between about 5 and about 10 mg/mL after the administering. In an aspect, the ferritin level in the TBI patient undergoing hemorrhagic shock is reduced to between about 10 and about 15 mg/mL after the administering.

Methods of the present disclosure provide for, and include, reducing the level of plasma atrial natriuretic peptide (ANP) in a TBI patient undergoing hemorrhagic shock, comprising administering to the patient oxygen reduced blood having an oxygen saturation (SO₂) of 20% or less prior to and during storage. In an aspect, the ANP level in the TBI patient undergoing hemorrhagic shock is reduced by between 10% to 20% after the administering. In an aspect, the ANP level in the TBI patient undergoing hemorrhagic shock is reduced by between 10% to 30% after the administering. In an aspect, the ANP level in the TBI patient undergoing hemorrhagic shock is reduced by between 10% to 40% after the administering. In an aspect, the ANP level in the TBI patient undergoing hemorrhagic shock is reduced by between 10% to 50% after the administering. In an aspect, the ANP level in the TBI patient undergoing hemorrhagic shock is reduced by between 20% to 30% after the administering. In an aspect, the ANP level in the TBI patient undergoing hemorrhagic shock is reduced by between 20% to 40% after the administering. In an aspect, the ANP level in the TBI patient undergoing hemorrhagic shock is reduced by between 20% to 50% after the administering. In an aspect, the ANP level in the TBI patient undergoing hemorrhagic shock is reduced by between 30% to 40% after the administering. In an aspect, the ANP level in the TBI patient undergoing hemorrhagic shock is reduced by between 30% to 50% after the administering. In an aspect, the ANP level in the TBI patient undergoing hemorrhagic shock is reduced by between 40% to 50% after the administering. In an aspect, the ANP level in the TBI patient undergoing hemorrhagic shock is reduced by at least 10%, 20%, 30%, 40%, or 50% after the administering.

In an aspect of the present disclosure, the level of plasma atrial natriuretic peptide (ANP) in a TBI patient undergoing hemorrhagic shock is reduced by between 1.5 and 10 fold after administering oxygen reduced blood having an oxygen saturation (SO₂) of 20% or less prior to and during storage to the patient. In an aspect, the ANP level in the TBI patient undergoing hemorrhagic shock is reduced by between 1.5 and 3 fold after the administering. In an aspect, the ANP level in the TBI patient undergoing hemorrhagic shock is reduced by between 1.5 and 5 fold after the administering. In an aspect, the ANP level in the TBI patient undergoing hemorrhagic shock is reduced by between 1.5 and 10 fold after the administering. In an aspect, the ANP level in the TBI patient undergoing hemorrhagic shock is reduced by between 3 and 5 fold after the administering. In an aspect, the ANP level in the TBI patient undergoing hemorrhagic shock is reduced by between 3 and 10 fold after the administering. In an aspect, the ANP level in the TBI patient undergoing hemorrhagic shock is reduced by between 5 and 10 fold after the administering.

Methods of the present disclosure provide for, and include, reducing the level of plasma atrial natriuretic peptide (ANP) in a TBI patient undergoing hemorrhagic shock, comprising administering to the patient oxygen reduced blood having an oxygen saturation (SO₂) of 20% or less prior to and during storage, compared to the level of ANP in a TBI patient undergoing hemorrhagic shock having been administered the same amount of non-oxygen reduced conventionally stored blood. In an aspect, the ANP level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the ANP level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of plasma atrial natriuretic peptide (ANP) in a TBI patient undergoing hemorrhagic shock to between about 5 and about 30 picograms per milliliter (pg/mL), comprising administering to the patient oxygen reduced blood having an oxygen saturation (SO₂) of 20% or less prior to and during storage. In an aspect, the ANP level in the TBI patient undergoing hemorrhagic shock is reduced to between about 25 and about 30 pg/mL after the administering. In an aspect, the ANP level in the TBI patient undergoing hemorrhagic shock is reduced to between about 20 and about 30 pg/mL after the administering. In an aspect, the ANP level in the TBI patient undergoing hemorrhagic shock is reduced to between about 15 and about 30 pg/mL after the administering. In an aspect, the ANP level in the TBI patient undergoing hemorrhagic shock is reduced to between about 10 and about 30 pg/mL after the administering. In an aspect, the ANP level in the TBI patient undergoing hemorrhagic shock is reduced to between about 5 and about 15 pg/mL after the administering. In an aspect, the ANP level in the TBI patient undergoing hemorrhagic shock is reduced to between about 5 and about 20 pg/mL after the administering.

Methods of the present disclosure provide for, and include, increasing the level of interleukin 10 (IL-10) in a TBI patient undergoing hemorrhagic shock, comprising administering to the patient oxygen reduced blood having an oxygen saturation (SO₂) of 20% or less prior to and during storage. In an aspect, the IL-10 level in the TBI patient undergoing hemorrhagic shock is increased by between 10% to 20% after the administering. In an aspect, the IL-10 level in the TBI patient undergoing hemorrhagic shock is increased by between 10% to 30% after the administering. In an aspect, the IL-10 level in the TBI patient undergoing hemorrhagic shock is increased by between 10% to 40% after the administering. In an aspect, the IL-10 level in the TBI patient undergoing hemorrhagic shock is increased by between 10% to 50% after the administering. In an aspect, the IL-10 level in the TBI patient undergoing hemorrhagic shock is increased by between 20% to 30% after the administering. In an aspect, the IL-10 level in the TBI patient undergoing hemorrhagic shock is increased by between 20% to 40% after the administering. In an aspect, the IL-10 level in the TBI patient undergoing hemorrhagic shock is increased by between 20% to 50% after the administering. In an aspect, the IL-10 level in the TBI patient undergoing hemorrhagic shock is increased by between 30% to 40% after the administering. In an aspect, the IL-10 level in the TBI patient undergoing hemorrhagic shock is increased by between 30% to 50% after the administering. In an aspect, the IL-10 level in the TBI patient undergoing hemorrhagic shock is increased by between 40% to 50% after the administering. In an aspect, the IL-10 level in the TBI patient undergoing hemorrhagic shock is increased by at least 10%, 20%, 30%, 40%, or 50% after the administering.

In an aspect of the present disclosure, the level of interleukin 10 (IL-10) in a TBI patient undergoing hemorrhagic shock is increased by between 1.5 and 10 fold after administering oxygen reduced blood having an oxygen saturation (SO₂) of 20% or less prior to and during storage to the patient. In an aspect, the IL-10 level in the TBI patient undergoing hemorrhagic shock is increased by between 1.5 and 3 fold after the administering. In an aspect, the IL-10 level in the TBI patient undergoing hemorrhagic shock is increased by between 1.5 and 5 fold after the administering. In an aspect, the IL-10 level in the TBI patient undergoing hemorrhagic shock is increased by between 1.5 and 10 fold after the administering. In an aspect, the IL-10 level in the TBI patient undergoing hemorrhagic shock is increased by between 3 and 5 fold after the administering. In an aspect, the IL-10 level in the TBI patient undergoing hemorrhagic shock is increased by between 3 and 10 fold after the administering. In an aspect, the IL-10 level in the TBI patient undergoing hemorrhagic shock is increased by between 5 and 10 fold after the administering.

Methods of the present disclosure provide for, and include, increasing the level of interleukin 10 (IL-10) in a TBI patient undergoing hemorrhagic shock, comprising administering to the patient oxygen reduced blood having an oxygen saturation (SO₂) of 20% or less prior to and during storage, compared to the level of IL-10 in a TBI patient undergoing hemorrhagic shock having been administered the same amount of non-oxygen reduced conventionally stored blood. In an aspect, the IL-10 level in the TBI patient undergoing hemorrhagic shock is increased by at least 5% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by between 5% to 10% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by between 5% to 20% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by between 5% to 30% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by between 10% to 20% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by between 10% to 30% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by between 20% to 30% after the administering, compared to a TBI patient undergoing hemorrhagic shock having been administered the same amount of non-oxygen reduced conventionally stored blood. In an aspect, the IL-10 level in the TBI patient undergoing hemorrhagic shock is increased by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, increasing the level of interleukin 10 (IL-10) in a TBI patient undergoing hemorrhagic shock to between about 3 and about 12 picograms per milliliter (pg/mL), comprising administering to the patient oxygen reduced blood having an oxygen saturation (SO₂) of 20% or less prior to and during storage. In an aspect, the IL-10 level in the TBI patient undergoing hemorrhagic shock is increased to between about 3 and about 6 pg/mL after the administering. In an aspect, the IL-10 level in the TBI patient undergoing hemorrhagic shock is increased to between about 3 and about 9 pg/mL after the administering. In an aspect, the IL-10 level in the TBI patient undergoing hemorrhagic shock is increased to between about 6 and about 9 pg/mL after the administering. In an aspect, the IL-10 level in the TBI patient undergoing hemorrhagic shock is increased to between about 9 and about 12 pg/mL after the administering.

Methods of the present disclosure provide for, and include, reducing the level of epinephrine in a TBI patient undergoing hemorrhagic shock, comprising administering to the patient oxygen reduced blood having an oxygen saturation (SO₂) of 20% or less prior to and during storage. In an aspect, the epinephrine level in the TBI patient undergoing hemorrhagic shock is reduced by between 10% to 20% after the administering. In an aspect, the epinephrine level in the TBI patient undergoing hemorrhagic shock is reduced by between 10% to 30% after the administering. In an aspect, the epinephrine level in the TBI patient undergoing hemorrhagic shock is reduced by between 10% to 40% after the administering. In an aspect, the epinephrine level in the TBI patient undergoing hemorrhagic shock is reduced by between 10% to 50% after the administering. In an aspect, the epinephrine level in the TBI patient undergoing hemorrhagic shock is reduced by between 20% to 30% after the administering. In an aspect, the epinephrine level in the TBI patient undergoing hemorrhagic shock is reduced by between 20% to 40% after the administering. In an aspect, the epinephrine level in the TBI patient undergoing hemorrhagic shock is reduced by between 20% to 50% after the administering. In an aspect, the epinephrine level in the TBI patient undergoing hemorrhagic shock is reduced by between 30% to 40% after the administering. In an aspect, the epinephrine level in the TBI patient undergoing hemorrhagic shock is reduced by between 30% to 50% after the administering. In an aspect, the epinephrine level in the TBI patient undergoing hemorrhagic shock is reduced by between 40% to 50% after the administering. In an aspect, the epinephrine level in the TBI patient undergoing hemorrhagic shock is reduced by at least 10%, 20%, 30%, 40%, or 50% after the administering.

In an aspect of the present disclosure, the level of epinephrine in a TBI patient undergoing hemorrhagic shock is reduced by between 1.5 and 10 fold after administering oxygen reduced blood having an oxygen saturation (SO₂) of 20% or less prior to and during storage to the patient. In an aspect, the epinephrine level in the TBI patient undergoing hemorrhagic shock is reduced by between 1.5 and 3 fold after the administering. In an aspect, the epinephrine level in the TBI patient undergoing hemorrhagic shock is reduced by between 1.5 and 5 fold after the administering. In an aspect, the epinephrine level in the TBI patient undergoing hemorrhagic shock is reduced by between 1.5 and 10 fold after the administering. In an aspect, the epinephrine level in the TBI patient undergoing hemorrhagic shock is reduced by between 3 and 5 fold after the administering. In an aspect, the epinephrine level in the TBI patient undergoing hemorrhagic shock is reduced by between 3 and 10 fold after the administering. In an aspect, the epinephrine level in the TBI patient undergoing hemorrhagic shock is reduced by between 5 and 10 fold after the administering.

Methods of the present disclosure provide for, and include, reducing the level of epinephrine in a TBI patient undergoing hemorrhagic shock, comprising administering to the patient oxygen reduced blood having an oxygen saturation (SO₂) of 20% or less prior to and during storage, compared to the level of epinephrine in a TBI patient undergoing hemorrhagic shock having been administered the same amount of non-oxygen reduced conventionally stored blood. In an aspect, the epinephrine level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the epinephrine level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of epinephrine in a TBI patient undergoing hemorrhagic shock to between about 5 and about 150 picograms per milliliter (pg/mL), comprising administering to the patient oxygen reduced blood having an oxygen saturation (SO₂) of 20% or less prior to and during storage. In an aspect, the epinephrine level in the TBI patient undergoing hemorrhagic shock is reduced to between about 100 and about 150 pg/mL after the administering. In an aspect, the epinephrine level in the TBI patient undergoing hemorrhagic shock is reduced to between about 50 and about 150 pg/mL after the administering. In an aspect, the epinephrine level in the TBI patient undergoing hemorrhagic shock is reduced to between about 50 and about 100 pg/mL after the administering. In an aspect, the epinephrine level in the TBI patient undergoing hemorrhagic shock is reduced to between about 5 and about 50 pg/mL after the administering.

Methods of the present disclosure provide for, and include, reducing the level of norepinephrine in a TBI patient undergoing hemorrhagic shock, comprising administering to the patient oxygen reduced blood having an oxygen saturation (SO₂) of 20% or less prior to and during storage. In an aspect, the norepinephrine level in the TBI patient undergoing hemorrhagic shock is reduced by between 10% to 20% after the administering. In an aspect, the norepinephrine level in the TBI patient undergoing hemorrhagic shock is reduced by between 10% to 30% after the administering. In an aspect, the norepinephrine level in the TBI patient undergoing hemorrhagic shock is reduced by between 10% to 40% after the administering. In an aspect, the norepinephrine level in the TBI patient undergoing hemorrhagic shock is reduced by between 10% to 50% after the administering. In an aspect, the norepinephrine level in the TBI patient undergoing hemorrhagic shock is reduced by between 20% to 30% after the administering. In an aspect, the norepinephrine level in the TBI patient undergoing hemorrhagic shock is reduced by between 20% to 40% after the administering. In an aspect, the norepinephrine level in the TBI patient undergoing hemorrhagic shock is reduced by between 20% to 50% after the administering. In an aspect, the norepinephrine level in the TBI patient undergoing hemorrhagic shock is reduced by between 30% to 40% after the administering. In an aspect, the norepinephrine level in the TBI patient undergoing hemorrhagic shock is reduced by between 30% to 50% after the administering. In an aspect, the norepinephrine level in the TBI patient undergoing hemorrhagic shock is reduced by between 40% to 50% after the administering. In an aspect, the norepinephrine level in the TBI patient undergoing hemorrhagic shock is reduced by at least 10%, 20%, 30%, 40%, or 50% after the administering.

In an aspect of the present disclosure, the level of norepinephrine in a TBI patient undergoing hemorrhagic shock is reduced by between 1.5 and 10 fold after administering oxygen reduced blood having an oxygen saturation (SO₂) of 20% or less prior to and during storage to the patient. In an aspect, the norepinephrine level in the TBI patient undergoing hemorrhagic shock is reduced by between 1.5 and 3 fold after the administering. In an aspect, the norepinephrine level in the TBI patient undergoing hemorrhagic shock is reduced by between 1.5 and 5 fold after the administering. In an aspect, the norepinephrine level in the TBI patient undergoing hemorrhagic shock is reduced by between 1.5 and 10 fold after the administering. In an aspect, the norepinephrine level in the TBI patient undergoing hemorrhagic shock is reduced by between 3 and 5 fold after the administering. In an aspect, the norepinephrine level in the TBI patient undergoing hemorrhagic shock is reduced by between 3 and 10 fold after the administering. In an aspect, the norepinephrine level in the TBI patient undergoing hemorrhagic shock is reduced by between 5 and 10 fold after the administering.

Methods of the present disclosure provide for, and include, reducing the level of norepinephrine in a TBI patient undergoing hemorrhagic shock, comprising administering to the patient oxygen reduced blood having an oxygen saturation (SO₂) of 20% or less prior to and during storage, compared to the level of norepinephrine in a TBI patient undergoing hemorrhagic shock having been administered the same amount of non-oxygen reduced conventionally stored blood. In an aspect, the norepinephrine level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the norepinephrine level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of norepinephrine in a TBI patient undergoing hemorrhagic shock to between about 50 and about 2000 picograms per milliliter (pg/mL), comprising administering to the patient oxygen reduced blood having an oxygen saturation (SO₂) of 20% or less prior to and during storage. In an aspect, the norepinephrine level in the TBI patient undergoing hemorrhagic shock is reduced to between about 1500 and about 2000 pg/mL after the administering. In an aspect, the norepinephrine level in the TBI patient undergoing hemorrhagic shock is reduced to between about 1000 and about 2000 pg/mL after the administering. In an aspect, the norepinephrine level in the TBI patient undergoing hemorrhagic shock is reduced to between about 500 and about 2000 pg/mL after the administering. In an aspect, the norepinephrine level in the TBI patient undergoing hemorrhagic shock is reduced to between about 50 and about 500 pg/mL after the administering. In an aspect, the norepinephrine level in the TBI patient undergoing hemorrhagic shock is reduced to between about 50 and about 1000 pg/mL after the administering. In an aspect, the norepinephrine level in the TBI patient undergoing hemorrhagic shock is reduced to between about 50 and about 1500 pg/mL after the administering.

Methods of the present disclosure provide for, and include, reducing the level of cortisol in a TBI patient undergoing hemorrhagic shock, comprising administering to the patient oxygen reduced blood having an oxygen saturation (SO₂) of 20% or less prior to and during storage. In an aspect, the cortisol level in the TBI patient undergoing hemorrhagic shock is reduced by between 10% to 20% after the administering. In an aspect, the cortisol level in the TBI patient undergoing hemorrhagic shock is reduced by between 10% to 30% after the administering. In an aspect, the cortisol level in the TBI patient undergoing hemorrhagic shock is reduced by between 10% to 40% after the administering. In an aspect, the cortisol level in the TBI patient undergoing hemorrhagic shock is reduced by between 10% to 50% after the administering. In an aspect, the cortisol level in the TBI patient undergoing hemorrhagic shock is reduced by between 20% to 30% after the administering. In an aspect, the cortisol level in the TBI patient undergoing hemorrhagic shock is reduced by between 20% to 40% after the administering. In an aspect, the cortisol level in the TBI patient undergoing hemorrhagic shock is reduced by between 20% to 50% after the administering. In an aspect, the cortisol level in the TBI patient undergoing hemorrhagic shock is reduced by between 30% to 40% after the administering. In an aspect, the cortisol level in the TBI patient undergoing hemorrhagic shock is reduced by between 30% to 50% after the administering. In an aspect, the cortisol level in the TBI patient undergoing hemorrhagic shock is reduced by between 40% to 50% after the administering. In an aspect, the cortisol level in the TBI patient undergoing hemorrhagic shock is reduced by at least 10%, 20%, 30%, 40%, or 50% after the administering.

In an aspect of the present disclosure, the level of cortisol in a TBI patient undergoing hemorrhagic shock is reduced by between 1.5 and 10 fold after administering oxygen reduced blood having an oxygen saturation (SO₂) of 20% or less prior to and during storage to the patient. In an aspect, the cortisol level in the TBI patient undergoing hemorrhagic shock is reduced by between 1.5 and 3 fold after the administering. In an aspect, the cortisol level in the TBI patient undergoing hemorrhagic shock is reduced by between 1.5 and 5 fold after the administering. In an aspect, the cortisol level in the TBI patient undergoing hemorrhagic shock is reduced by between 1.5 and 10 fold after the administering. In an aspect, the cortisol level in the TBI patient undergoing hemorrhagic shock is reduced by between 3 and 5 fold after the administering. In an aspect, the cortisol level in the TBI patient undergoing hemorrhagic shock is reduced by between 3 and 10 fold after the administering. In an aspect, the cortisol level in the TBI patient undergoing hemorrhagic shock is reduced by between 5 and 10 fold after the administering.

Methods of the present disclosure provide for, and include, reducing the level of cortisol in a TBI patient undergoing hemorrhagic shock, comprising administering to the patient oxygen reduced blood having an oxygen saturation (SO₂) of 20% or less prior to and during storage, compared to the level of cortisol in a TBI patient undergoing hemorrhagic shock having been administered the same amount of non-oxygen reduced conventionally stored blood. In an aspect, the cortisol level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the cortisol level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of cortisol in a TBI patient undergoing hemorrhagic shock to between about 2 and about 25 milligrams per deciliter (mg/dL), comprising administering to the patient oxygen reduced blood having an oxygen saturation (SO₂) of 20% or less prior to and during storage. In an aspect, the cortisol level in the TBI patient undergoing hemorrhagic shock is reduced to between about 20 and about 25 mg/dL after the administering. In an aspect, the cortisol level in the TBI patient undergoing hemorrhagic shock is reduced to between about 15 and about 25 mg/dL after the administering. In an aspect, the cortisol level in the TBI patient undergoing hemorrhagic shock is reduced to between about 10 and about 25 mg/dL after the administering. In an aspect, the cortisol level in the TBI patient undergoing hemorrhagic shock is reduced to between about 5 and about 25 mg/dL after the administering. In an aspect, the cortisol level in the TBI patient undergoing hemorrhagic shock is reduced to between about 5 and about 15 mg/dL after the administering. In an aspect, the cortisol level in the TBI patient undergoing hemorrhagic shock is reduced to between about 2 and about 10 mg/dL after the administering.

Methods of the present disclosure provide for, and include, reducing the level of urine creatinine in a TBI patient undergoing hemorrhagic shock, comprising administering to the patient oxygen reduced blood having an oxygen saturation (SO₂) of 20% or less prior to and during storage. In an aspect, the urine creatinine level in the TBI patient undergoing hemorrhagic shock is reduced by between 10% to 20% after the administering. In an aspect, the urine creatinine level in the TBI patient undergoing hemorrhagic shock is reduced by between 10% to 30% after the administering. In an aspect, the urine creatinine level in the TBI patient undergoing hemorrhagic shock is reduced by between 10% to 40% after the administering. In an aspect, the urine creatinine level in the TBI patient undergoing hemorrhagic shock is reduced by between 10% to 50% after the administering. In an aspect, the urine creatinine level in the TBI patient undergoing hemorrhagic shock is reduced by between 20% to 30% after the administering. In an aspect, the urine creatinine level in the TBI patient undergoing hemorrhagic shock is reduced by between 20% to 40% after the administering. In an aspect, the urine creatinine level in the TBI patient undergoing hemorrhagic shock is reduced by between 20% to 50% after the administering. In an aspect, the urine creatinine level in the TBI patient undergoing hemorrhagic shock is reduced by between 30% to 40% after the administering. In an aspect, the urine creatinine level in the TBI patient undergoing hemorrhagic shock is reduced by between 30% to 50% after the administering. In an aspect, the urine creatinine level in the TBI patient undergoing hemorrhagic shock is reduced by between 40% to 50% after the administering. In an aspect, the urine creatinine level in the TBI patient undergoing hemorrhagic shock is reduced by at least 10%, 20%, 30%, 40%, or 50% after the administering.

In an aspect of the present disclosure, the level of urine creatinine in a TBI patient undergoing hemorrhagic shock is reduced by between 1.5 and 10 fold after administering oxygen reduced blood having an oxygen saturation (SO₂) of 20% or less prior to and during storage to the patient. In an aspect, the urine creatinine level in the TBI patient undergoing hemorrhagic shock is reduced by between 1.5 and 3 fold after the administering. In an aspect, the urine creatinine level in the TBI patient undergoing hemorrhagic shock is reduced by between 1.5 and 5 fold after the administering. In an aspect, the urine creatinine level in the TBI patient undergoing hemorrhagic shock is reduced by between 1.5 and 10 fold after the administering. In an aspect, the urine creatinine level in the TBI patient undergoing hemorrhagic shock is reduced by between 3 and 5 fold after the administering. In an aspect, the urine creatinine level in the TBI patient undergoing hemorrhagic shock is reduced by between 3 and 10 fold after the administering. In an aspect, the urine creatinine level in the TBI patient undergoing hemorrhagic shock is reduced by between 5 and 10 fold after the administering.

Methods of the present disclosure provide for, and include, reducing the level of urine creatinine in a TBI patient undergoing hemorrhagic shock, comprising administering to the patient oxygen reduced blood having an oxygen saturation (SO₂) of 20% or less prior to and during storage, compared to the level of urine creatinine in a TBI patient undergoing hemorrhagic shock having been administered the same amount of non-oxygen reduced conventionally stored blood. In an aspect, the urine creatinine level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by between 5% to 10% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by between 5% to 20% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by between 5% to 30% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by between 10% to 20% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by between 20% to 30% after the administering, compared to a TBI patient undergoing hemorrhagic shock having been administered the same amount of non-oxygen reduced conventionally stored blood. In an aspect, the urine creatinine level in the TBI patient undergoing hemorrhagic shock is reduced by at least 5%, 10%, 20%, or 30% after the administering, compared to a TBI patient undergoing hemorrhagic shock having been administered the same amount of non-oxygen reduced conventionally stored blood.

Methods of the present disclosure provide for, and include, reducing the level of urine creatinine in a TBI patient undergoing hemorrhagic shock to between about 10 and about 350 milligrams per deciliter (mg/dL), comprising administering to the patient oxygen reduced blood having an oxygen saturation (SO₂) of 20% or less prior to and during storage. In an aspect, the urine creatinine level in the TBI patient undergoing hemorrhagic shock is reduced to between about 300 and about 350 mg/dL after the administering. In an aspect, the urine creatinine level in the TBI patient undergoing hemorrhagic shock is reduced to between about 250 and about 350 mg/dL after the administering. In an aspect, the urine creatinine level in the TBI patient undergoing hemorrhagic shock is reduced to between about 200 and about 350 mg/dL after the administering. In an aspect, the urine creatinine level in the TBI patient undergoing hemorrhagic shock is reduced to between about 100 and about 350 mg/dL after the administering. In an aspect, the urine creatinine level in the TBI patient undergoing hemorrhagic shock is reduced to between about 50 and about 350 mg/dL after the administering.

Methods of the present disclosure provide for, and include, increasing tissue oxygen saturation in one or more vital organs of a TBI patient undergoing hemorrhagic shock, comprising administering to the patient oxygen reduced blood having an oxygen saturation (SO₂) of 20% or less prior to and during storage. In an aspect, the one or more vital organs are selected from the group consisting of the kidney, the liver, the lungs, the spleen, and the heart. In an aspect, the tissue oxygen saturation in one or more vital organs of the TBI patient undergoing hemorrhagic shock is increased by at least 10% after the administering. In an aspect, the tissue oxygen saturation in one or more vital organs of the TBI patient undergoing hemorrhagic shock is increased by at least 25% after the administering. In an aspect, the tissue oxygen saturation in one or more vital organs of the TBI patient undergoing hemorrhagic shock is increased by between 10% to 20% after the administering. In an aspect, the tissue oxygen saturation in one or more vital organs of the TBI patient undergoing hemorrhagic shock is increased by between 10% to 30% after the administering. In an aspect, the tissue oxygen saturation in one or more vital organs of the TBI patient undergoing hemorrhagic shock is increased by between 10% to 40% after the administering. In an aspect, the tissue oxygen saturation in one or more vital organs of the TBI patient undergoing hemorrhagic shock is increased by between 10% to 50% after the administering. In an aspect, the tissue oxygen saturation in one or more vital organs of the TBI patient undergoing hemorrhagic shock is increased by between 20% to 30% after the administering. In an aspect, the tissue oxygen saturation in one or more vital organs of the TBI patient undergoing hemorrhagic shock is increased by between 20% to 40% after the administering. In an aspect, the tissue oxygen saturation in one or more vital organs of the TBI patient undergoing hemorrhagic shock is increased by between 20% to 50% after the administering. In an aspect, the tissue oxygen saturation in one or more vital organs of the TBI patient undergoing hemorrhagic shock is increased by between 30% to 40% after the administering. In an aspect, the tissue oxygen saturation in one or more vital organs of the TBI patient undergoing hemorrhagic shock is increased by between 30% to 50% after the administering. In an aspect, the tissue oxygen saturation in one or more vital organs of the TBI patient undergoing hemorrhagic shock is increased by between 40% to 50% after the administering. In an aspect, the tissue oxygen saturation in one or more vital organs of the TBI patient undergoing hemorrhagic shock is increased by at least 10%, 20%, 30%, 40%, or 50% after the administering. In an aspect, the tissue oxygen saturation in each of the one or more vital organs of a TBI patient undergoing hemorrhagic shock is measured by administering an effective amount of pimonidazole to the patient and detecting the distribution of the pimonidazole in the one or more vital organs of the patient.

In an aspect, the tissue oxygen saturation in each of one or more vital organs of a TBI patient undergoing hemorrhagic shock is increased by between 1.5 and 10 fold after administering oxygen reduced blood having an oxygen saturation (SO₂) of 20% or less prior to and during storage to the patient. In an aspect, the one or more vital organs are selected from the group consisting of the kidney, the liver, the lungs, the spleen, and the heart. In an aspect, the tissue oxygen saturation in each of one or more vital organs in a TBI patient undergoing hemorrhagic shock is increased by between 1.5 and 3 fold after the administering. In an aspect, the tissue oxygen saturation in each of one or more vital organs in a TBI patient undergoing hemorrhagic shock is increased by between 1.5 and 5 fold after the administering. In an aspect, the tissue oxygen saturation in each of one or more vital organs in a TBI patient undergoing hemorrhagic shock is increased by between 1.5 and 10 fold after the administering. In an aspect, the tissue oxygen saturation in each of one or more vital organs in a TBI patient undergoing hemorrhagic shock is increased by between 3 and 5 fold after the administering. In an aspect, the tissue oxygen saturation in each of one or more vital organs in a TBI patient undergoing hemorrhagic shock is increased by between 3 and 10 fold after the administering. In an aspect, the tissue oxygen saturation in each of one or more vital organs in a TBI patient undergoing hemorrhagic shock is increased by between 5 and 10 fold after the administering.

Methods of the present disclosure provide for, and include, increasing the tissue oxygen saturation in one or more vital organs of a TBI patient undergoing hemorrhagic shock, comprising administering to the patient oxygen reduced blood having an oxygen saturation (SO₂) of 20% or less prior to and during storage, compared to the tissue oxygen saturation in the same one or more vital organs of a TBI patient undergoing hemorrhagic shock having been administered the same amount of non-oxygen reduced conventionally stored blood. In an aspect, the one or more vital organs are selected from the group consisting of the kidney, the liver, the lungs, the spleen, and the heart. In an aspect, the tissue oxygen saturation in each of one or more vital organs of the TBI patient undergoing hemorrhagic shock is increased by at least 10% after the administering, compared to the tissue oxygen saturation in each of the same one or more vital organs of a TBI patient undergoing hemorrhagic shock having been administered in the same amount of non-oxygen reduced conventionally stored blood. In an aspect, the tissue oxygen saturation in each of one or more vital organs of the TBI patient undergoing hemorrhagic shock is increased by at least 25% after the administering, compared to a TBI patient undergoing hemorrhagic shock having been administered in the same amount of non-oxygen reduced conventionally stored blood. In an aspect, the tissue oxygen saturation in each of one or more vital organs of the TBI patient undergoing hemorrhagic shock is increased by between 10% to 20% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 10% to 30% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 10% to 40% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 10% to 50% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 20% to 30% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 20% to 40% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 20% to 50% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 30% to 40% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 30% to 50% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by between 40% to 50% after the administering, compared to a TBI patient undergoing hemorrhagic shock having been administered the same amount of non-oxygen reduced conventionally stored blood. In an aspect, the tissue oxygen saturation in each of one or more vital organs of the TBI patient undergoing hemorrhagic shock is increased by at least 10%, 20%, 30%, 40%, or 50% after the administering, compared to a TBI patient undergoing hemorrhagic shock having been administered the same amount of non-oxygen reduced conventionally stored blood.

Methods of the present disclosure provide for, and include, reducing the level of blood lactate in a TBI patient undergoing hemorrhagic shock, comprising administering to the patient oxygen reduced blood having an oxygen saturation (SO₂) of 20% or less prior to and during storage. In an aspect, the TBI patient undergoing hemorrhagic shock has a blood lactate level that is greater than 3.0 millimoles per liter (mmol/L) prior to the administering. In an aspect, the TBI patient undergoing hemorrhagic shock has a blood lactate level that is greater than 4.0 mmol/L prior to the administering. In an aspect, the TBI patient undergoing hemorrhagic shock has a blood lactate level that is greater than 5.0 mmol/L prior to the administering.

In an aspect, the blood lactate level in a TBI patient undergoing hemorrhagic shock is reduced by between 10% to 30% after administering oxygen reduced blood having an oxygen saturation (SO₂) of 20% or less prior to and during storage to the patient. In an aspect, the blood lactate level in the TBI patient undergoing hemorrhagic shock is reduced by between 10% to 60% after the administering. In an aspect, the blood lactate level in the TBI patient undergoing hemorrhagic shock is reduced by between 10% to 90% after the administering. In an aspect, the blood lactate level in the TBI patient undergoing hemorrhagic shock is reduced by between 20% to 40% after the administering. In an aspect, the blood lactate level in the TBI patient undergoing hemorrhagic shock is reduced by between 30% to 60% after the administering. In an aspect, the blood lactate level in the TBI patient undergoing hemorrhagic shock is reduced by between 30% to 90% after the administering. In an aspect, the blood lactate level in the TBI patient undergoing hemorrhagic shock is reduced by between 60% to 90% after the administering. In an aspect, the blood lactate level in the TBI patient undergoing hemorrhagic shock is reduced by greater than 90% after administering. In an aspect, the blood lactate level in the TBI patient undergoing hemorrhagic shock is reduced by at least 10%, 20%, 30%, 40%, 50%, 60%, 70%, 80%, or 90% after the administering. In an aspect, the blood lactate level in the TBI patient undergoing hemorrhagic shock is reduced by between 10% to 20%, 20% to 30%, 30% to 40%, 40% to 50%, 50% to 60%, 60% to 70%, 70% to 80%, or 80% to 90% after the administering.

In an aspect of the present disclosure, the level of blood lactate in a TBI patient undergoing hemorrhagic shock is reduced by between 1.5 and 10 fold after administering oxygen reduced blood having an oxygen saturation (SO₂) of 20% or less prior to and during storage to the patient. In an aspect, the blood lactate level in the TBI patient undergoing hemorrhagic shock is reduced by between 1.5 and 3 fold after the administering. In an aspect, the blood lactate level in the TBI patient undergoing hemorrhagic shock is reduced by between 1.5 and 5 fold after the administering. In an aspect, the blood lactate level in the TBI patient undergoing hemorrhagic shock is reduced by between 1.5 and 10 fold after the administering. In an aspect, the blood lactate level in the TBI patient undergoing hemorrhagic shock is reduced by between 3 and 5 fold after the administering. In an aspect, the blood lactate level in the TBI patient undergoing hemorrhagic shock is reduced by between 3 and 10 fold after the administering. In an aspect, the blood lactate level in the TBI patient undergoing hemorrhagic shock is reduced by between 5 and 10 fold after the administering.

Methods of the present disclosure provide for, and include, reducing the level of blood lactate in a TBI patient undergoing hemorrhagic shock, comprising administering to the patient oxygen reduced blood having an oxygen saturation (SO₂) of 20% or less prior to and during storage, compared to the level of blood lactate in a TBI patient undergoing hemorrhagic shock having been administered the same amount of non-oxygen reduced conventionally stored blood. In an aspect, the blood lactate level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by at least 15% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 15% to 30% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 15% to 60% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 15% to 90% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 10% to 30% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 10% to 60% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 10% to 90% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 30% to 60% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 30% to 90% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by between 60% to 90% after the administering, compared to a TBI patient undergoing hemorrhagic shock having been administered the same amount of non-oxygen reduced conventionally stored blood. In an aspect, the blood lactate level in the TBI patient undergoing hemorrhagic shock is reduced by at least 10%, 15%, 20%, 30%, 40%, 50%, 60%, 70%, 80%, or 90% after the administering, compared to a TBI patient undergoing hemorrhagic shock having been administered the same amount of non-oxygen reduced conventionally stored blood.

Methods of the present disclosure provide for, and include, reducing the blood lactate level of a TBI patient undergoing hemorrhagic shock to between about 0.5 and about 3.0 millimoles per liter (mmol/L), comprising administering to the patient oxygen reduced blood having an oxygen saturation (SO₂) of 20% or less prior to and during storage. In an aspect, the blood lactate level in the TBI patient undergoing hemorrhagic shock is reduced to between about 0.9 and about 2 mmol/L after the administering. In an aspect, the blood lactate level in the TBI patient undergoing hemorrhagic shock is reduced to between about 0.9 and about 1.7 mmol/L after the administering. In an aspect, the blood lactate level in the TBI patient undergoing hemorrhagic shock is reduced to between about 1.4 and about 2.4 mmol/L after the administering. In an aspect, the blood lactate level in the TBI patient undergoing hemorrhagic shock is reduced to between about 1.7 and about 2.5 mmol/L after the administering. In an aspect, the blood lactate level in the TBI patient undergoing hemorrhagic shock is reduced to less than about 2.5 mmol/L after the administering. In an aspect, the lactate level in the TBI patient undergoing hemorrhagic shock is reduced to less than about 2.0 mmol/L after the administering. In an aspect, the lactate level in the TBI patient undergoing hemorrhagic shock is reduced to less than about 1.5 mmol/L after the administering. In an aspect, the lactate level in the TBI patient undergoing hemorrhagic shock is reduced to less than about 1.0 mmol/L after the administering. In an aspect, the lactate level in the TBI patient undergoing hemorrhagic shock is reduced to between about 0.5 and about 1.0 mmol/L after the administering.

Methods of the present disclosure provide for, and include, reducing the blood lactate level in a TBI patient undergoing hemorrhagic shock to less than 4 millimoles per liter (mmol/L), comprising administering to the patient oxygen reduced blood having an oxygen saturation (SO₂) of 20% or less prior to and during storage. In an aspect, the blood lactate level in the TBI patient undergoing hemorrhagic shock is reduced to less than 3 mmol/L after the administering. In an aspect, the blood lactate level in the TBI patient undergoing hemorrhagic shock is reduced to less than 2.5 mmol/L after the administering. In an aspect, the blood lactate level in the TBI patient undergoing hemorrhagic shock is reduced to less than 2.3 mmol/L after the administering. In an aspect, the blood lactate level in the TBI patient undergoing hemorrhagic shock is reduced to less than 2 mmol/L after the administering. In an aspect, the blood lactate level in the TBI patient undergoing hemorrhagic shock is reduced to less than 1.5 mmol/L after the administering. In an aspect, the blood lactate level in the TBI patient undergoing hemorrhagic shock is reduced to less than 1 mmol/L after the administering.

Blood glucose level is known to be a predictor for outcome in several disease patterns and particularly in trauma patients, including patients with traumatic brain injury (TBI) accompanied by hemorrhagic shock. Trauma patients, including patients with TBI, are more prone to poor outcome due to hyperglycemia than other critically ill patients. See Kreutziger et al., “Admission blood glucose predicted hemorrhagic shock in multiple trauma patients,” Injury, 46:15-20 (2015) (hereby incorporated by reference in its entirety). Studies evaluating the relationship of early hyperglycemia and trauma patients examined early hyperglycemia at three possible cutoffs: glucose≥110 mg/dL, glucose≥150 mg/dL, and glucose≥200 mg/dL. See Laird et al., “Relationship of early hyperglycemia to mortality in trauma patients,” J Trauma, 56:1058-62 (2004) (hereby incorporated by reference in its entirety). In an aspect, a TBI patient undergoing hemorrhagic shock has a blood glucose level that is greater than 120 milligrams per deciliter (mg/dL) prior to administering oxygen reduced blood to the patient.

Methods of the present disclosure provide for, and include, reducing the blood glucose level in a TBI patient undergoing hemorrhagic shock, comprising administering to the patient oxygen reduced blood having an oxygen saturation (SO₂) of 20% or less prior to and during storage. In an aspect, the blood glucose level in the TBI patient undergoing hemorrhagic shock is reduced by between 10% to 30% after the administering. In an aspect, the blood glucose level in the TBI patient undergoing hemorrhagic shock is reduced by between 10% to 60% after the administering. In an aspect, the blood glucose level in the TBI patient undergoing hemorrhagic shock is reduced by between 10% to 90% after the administering. In an aspect, the blood glucose level in the TBI patient undergoing hemorrhagic shock is reduced by between 20% to 40% after the administering. In an aspect, the blood glucose level in the TBI patient undergoing hemorrhagic shock is reduced by between 30% to 60% after the administering. In an aspect, the blood glucose level in the TBI patient undergoing hemorrhagic shock is reduced by between 30% to 90% after the administering. In an aspect, the blood glucose level in the TBI patient undergoing hemorrhagic shock is reduced by between 60% to 90% after the administering. In an aspect, the blood glucose level in the TBI patient undergoing hemorrhagic shock is reduced by greater than 90% after administering. In an aspect, the blood glucose level in the TBI patient undergoing hemorrhagic shock is reduced by at least 10%, 20%, 30%, 40%, 50%, 60%, 70%, 80%, or 90% after the administering. In an aspect, the blood glucose level in the TBI patient undergoing hemorrhagic shock is reduced by between 10% to 20%, 20% to 30%, 30% to 40%, 40% to 50%, 50% to 60%, 60% to 70%, 70% to 80%, or 80% to 90% after the administering.

In an aspect of the present disclosure, the blood glucose level in a TBI patient undergoing hemorrhagic shock is reduced by between 1.5 and 10 fold after administering oxygen reduced blood having an oxygen saturation (SO₂) of 20% or less prior to and during storage to the patient. In an aspect, the blood glucose level in the TBI patient undergoing hemorrhagic shock is reduced by between 1.5 and 3 fold after the administering. In an aspect, the blood glucose level in the TBI patient undergoing hemorrhagic shock is reduced by between 1.5 and 5 fold after the administering. In an aspect, the blood glucose level in the TBI patient undergoing hemorrhagic shock is reduced by between 1.5 and 10 fold after the administering. In an aspect, the blood glucose level in the TBI patient undergoing hemorrhagic shock is reduced by between 3 and 5 fold after the administering. In an aspect, the blood glucose level in the TBI patient undergoing hemorrhagic shock is reduced by between 3 and 10 fold after the administering. In an aspect, the blood glucose level in the TBI patient undergoing hemorrhagic shock is reduced by between 5 and 10 fold after the administering.

Methods of the present disclosure provide for, and include, reducing the blood glucose level in a TBI patient undergoing hemorrhagic shock, comprising administering to the patient oxygen reduced blood having an oxygen saturation (SO₂) of 20% or less prior to and during storage, compared to the blood glucose level in a TBI patient undergoing hemorrhagic shock having been administered the same amount of non-oxygen reduced conventionally stored blood. In an aspect, the blood glucose level in the TBI patient undergoing hemorrhagic shock is reduced by at least 5% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by between 5% to 90% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by between 10% to 50% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by between 20% to 40% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by between 50% to 90% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by between 60% to 90% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by at least 10%, 15%, 20%, 30%, 40%, 50%, 60%, 70%, 80%, or 90% after the administering, compared to a TBI patient undergoing hemorrhagic shock having been administered the same amount of non-oxygen reduced conventionally stored blood. In an aspect, the blood glucose level in the TBI patient undergoing hemorrhagic shock is reduced by at least 30%, at least 40%, at least 50%, at least 60%, at least 70%, at least 80%, or at least 90% after the administering, compared to a TBI patient undergoing hemorrhagic shock having been administered the same amount of non-oxygen reduced conventionally stored blood.

Methods of the present disclosure provide for, and include, reducing the blood glucose level in a TBI patient undergoing hemorrhagic shock to between about 70 and about 120 milligrams per deciliter (mg/dL), comprising administering to the patient oxygen reduced blood having an oxygen saturation (SO₂) of 20% or less prior to and during storage. In an aspect, the blood glucose level in the TBI patient undergoing hemorrhagic shock is reduced to between about 70 and about 110 mg/dL after the administering. In another aspect, the blood glucose level in the TBI patient undergoing hemorrhagic shock is reduced to between about 70 and about 100 mg/dL after the administering. In another aspect, the blood glucose level in the TBI patient undergoing hemorrhagic shock is reduced to between about 90 and about 120 mg/dL after the administering. In another aspect, the blood glucose level in the TBI patient undergoing hemorrhagic shock is reduced to between about 90 and about 100 mg/dL after the administering.

Methods of the present disclosure provide for, and include, reducing the blood glucose level in a TBI patient undergoing hemorrhagic shock to less than 120 milligrams per deciliter (mg/dL), comprising administering to the patient oxygen reduced blood having an oxygen saturation (SO₂) of 20% or less prior to and during storage. In an aspect, the blood glucose level in the TBI patient undergoing hemorrhagic shock is reduced to less than 110 mg/dL after the administering. In an aspect, the blood glucose level in the TBI patient undergoing hemorrhagic shock is reduced to less than 100 mg/dL after the administering. In an aspect, the blood glucose level in the TBI patient undergoing hemorrhagic shock is reduced to less than 200 mg/dL after the administering. In an aspect, the blood glucose level in the TBI patient undergoing hemorrhagic shock is reduced to less than 90 mg/dL after the administering. In an aspect, the blood glucose level in the TBI patient undergoing hemorrhagic shock is reduced to less than 80 mg/dL after the administering.

Methods of the present disclosure provide for, and include, increasing the hematocrit of a TBI patient undergoing hemorrhagic shock, comprising administering to the patient oxygen reduced blood having an oxygen saturation (SO₂) of 20% or less prior to and during storage. In an aspect, the hematocrit of the TBI patient undergoing hemorrhagic shock is less than 35% prior to the administering. In an aspect, the hematocrit of the TBI patient undergoing hemorrhagic shock is less than 25% prior to the administering. In an aspect, the hematocrit of the TBI patient undergoing hemorrhagic shock is less than 15% prior to the administering. In an aspect, the hematocrit of the TBI patient undergoing hemorrhagic shock is increased by at least 10% after the administering. In an aspect, the hematocrit of the TBI patient undergoing hemorrhagic shock is increased by between 10% to 20% after the administering. In an aspect, the hematocrit of the TBI patient undergoing hemorrhagic shock is increased by between 10% to 30% after the administering. In an aspect, the hematocrit of the TBI patient undergoing hemorrhagic shock is increased by between 10% to 40% after the administering. In an aspect, the hematocrit of the TBI patient undergoing hemorrhagic shock is increased by between 10% to 50% after the administering. In an aspect, the hematocrit of the TBI patient undergoing hemorrhagic shock is increased by between 20% to 30% after the administering. In an aspect, the hematocrit of the TBI patient undergoing hemorrhagic shock is increased by between 20% to 40% after the administering. In an aspect, the hematocrit of the TBI patient undergoing hemorrhagic shock is increased by between 20% to 50% after the administering. In an aspect, the hematocrit of the TBI patient undergoing hemorrhagic shock is increased by between 30% to 40% after the administering. In an aspect, the hematocrit of the TBI patient undergoing hemorrhagic shock is increased by between 30% to 50% after the administering. In an aspect, the hematocrit of the TBI patient undergoing hemorrhagic shock is increased by between 40% to 50% after the administering. In an aspect, the hematocrit of the TBI patient undergoing hemorrhagic shock is increased by at least 10%, 20%, 30%, 40%, or 50% after the administering.

In an aspect of the present disclosure, the hematocrit of a TBI patient undergoing hemorrhagic shock is increased by between 1.5 and 10 fold after administering oxygen reduced blood having an oxygen saturation (SO₂) of 20% or less prior to and during storage to the patient. In an aspect, the hematocrit of the TBI patient undergoing hemorrhagic shock is increased by between 1.5 and 3 fold after the administering. In an aspect, the hematocrit of the TBI patient undergoing hemorrhagic shock is increased by between 1.5 and 5 fold after the administering. In an aspect, the hematocrit of the TBI patient undergoing hemorrhagic shock is increased by between 1.5 and 10 fold after the administering. In an aspect, the hematocrit of the TBI patient undergoing hemorrhagic shock is increased by between 3 and 5 fold after the administering. In an aspect, the hematocrit of the TBI patient undergoing hemorrhagic shock is increased by between 3 and 10 fold after the administering. In an aspect, the hematocrit of the TBI patient undergoing hemorrhagic shock is increased by between 5 and 10 fold after the administering.

Methods of the present disclosure provide for, and include, increasing the hematocrit of a TBI patient undergoing hemorrhagic shock, comprising administering to the patient oxygen reduced blood having an oxygen saturation (SO₂) of 20% or less prior to and during storage, compared to the hematocrit of a TBI patient undergoing hemorrhagic shock having been administered the same amount of non-oxygen reduced conventionally stored blood. In an aspect, the hematocrit of the TBI patient undergoing hemorrhagic shock is increased by at least 10% after the administering, compared to a TBI patient undergoing hemorrhagic shock having been administered the same amount of non-oxygen reduced conventionally stored blood. In an aspect, the hematocrit of the TBI patient undergoing hemorrhagic shock is increased by between 10% to 20% after the administering, compared to a TBI patient undergoing hemorrhagic shock having been administered the same amount of non-oxygen reduced conventionally stored blood. In an aspect, the hematocrit of the TBI patient undergoing hemorrhagic shock is increased by between 10% to 30% after the administering, compared to a TBI patient undergoing hemorrhagic shock having been administered the same amount of non-oxygen reduced conventionally stored blood. In an aspect, the hematocrit of the TBI patient undergoing hemorrhagic shock is increased by between 10% to 40% after the administering, compared to a TBI patient undergoing hemorrhagic shock having been administered the same amount of non-oxygen reduced conventionally stored blood. In an aspect, the hematocrit of the TBI patient undergoing hemorrhagic shock is increased by between 20% to 30% after the administering, compared to a TBI patient undergoing hemorrhagic shock having been administered the same amount of non-oxygen reduced conventionally stored blood. In an aspect, the hematocrit of the TBI patient undergoing hemorrhagic shock is increased by between 20% to 40% after the administering, compared to a TBI patient undergoing hemorrhagic shock having been administered the same amount of non-oxygen reduced conventionally stored blood. In an aspect, the hematocrit of the TBI patient undergoing hemorrhagic shock is increased by at least 5%, 10%, 20%, 30%, or 40% after the administering, compared to a TBI patient undergoing hemorrhagic shock having been administered the same amount of non-oxygen reduced conventionally stored blood.

Methods of the present disclosure provide for, and include, increasing the hematocrit of a TBI patient undergoing hemorrhagic shock to between about 35% and about 55%, comprising administering to the patient oxygen reduced blood having an oxygen saturation (SO₂) of 20% or less prior to and during storage. In an aspect, the hematocrit of the TBI patient undergoing hemorrhagic shock is increased to between about 40% and about 50% after the administering. In an aspect, the hematocrit of the TBI patient undergoing hemorrhagic shock is increased to between about 40% and about 55% after the administering. In an aspect, the hematocrit of the TBI patient undergoing hemorrhagic shock is increased to between about 35% to 45% after the administering.

Methods of the present disclosure provide for, and include, increasing the cardiac output (CO) of a TBI patient undergoing hemorrhagic shock, comprising administering to the patient oxygen reduced blood having an oxygen saturation (SO₂) of 20% or less prior to and during storage. In an aspect, the cardiac output of the TBI patient undergoing hemorrhagic shock is increased by at least 2% after the administering. In an aspect, the cardiac output of the TBI patient undergoing hemorrhagic shock is increased by between 2% to 5% after the administering. In an aspect, the cardiac output of the TBI patient undergoing hemorrhagic shock is increased by between 2% to 10% after the administering. In an aspect, the cardiac output of the TBI patient undergoing hemorrhagic shock is increased by between 2% to 15% after the administering. In an aspect, the cardiac output of the TBI patient undergoing hemorrhagic shock is increased by between 2% to 20% after the administering. In an aspect, the cardiac output of the TBI patient undergoing hemorrhagic shock is increased by between 5% to 10% after the administering. In an aspect, the cardiac output of the TBI patient undergoing hemorrhagic shock is increased by between 5% to 15% after the administering. In an aspect, the cardiac output of the TBI patient undergoing hemorrhagic shock is increased by between 5% to 20% after the administering. In an aspect, the cardiac output of the TBI patient undergoing hemorrhagic shock is increased by between 10% to 20% after the administering. In an aspect, the cardiac output of the TBI patient undergoing hemorrhagic shock is increased by between 15% to 20% after the administering. In an aspect, the cardiac output of the TBI patient undergoing hemorrhagic shock is increased by at least 2%, 5%, 10%, 15%, or 20% after the administering.

In an aspect of the present disclosure, the cardiac output (CO) of a TBI patient undergoing hemorrhagic shock is increased by between 1.5 and 10 fold after administering oxygen reduced blood having an oxygen saturation (SO₂) of 20% or less prior to and during storage to the patient. In an aspect, the cardiac output of the TBI patient undergoing hemorrhagic shock is increased by between 1.5 and 3 fold after the administering. In an aspect, the cardiac output of the TBI patient undergoing hemorrhagic shock is increased by between 1.5 and 5 fold after the administering. In an aspect, the cardiac output of the TBI patient undergoing hemorrhagic shock is increased by between 1.5 and 10 fold after the administering. In an aspect, the cardiac output of the TBI patient undergoing hemorrhagic shock is increased by between 3 and 5 fold after the administering. In an aspect, the cardiac output of the TBI patient undergoing hemorrhagic shock is increased by between 3 and 10 fold after the administering. In an aspect, the cardiac output of the TBI patient undergoing hemorrhagic shock is increased by between 5 and 10 fold after the administering.

Methods of the present disclosure provide for, and include, increasing the cardiac output (CO) of a TBI patient undergoing hemorrhagic shock, comprising administering to the patient oxygen reduced blood having an oxygen saturation (SO₂) of 20% or less prior to and during storage, compared to the cardiac output of a TBI patient undergoing hemorrhagic shock having been administered the same amount of non-oxygen reduced conventionally stored blood. In an aspect, the cardiac output of the TBI patient undergoing hemorrhagic shock is increased by at least 2% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 2% to 5% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 2% to 10% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 2% to 15% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 2% to 20% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 5% to 20% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 5% to 10% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 5% to 15% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 5% to 20% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 10% to 20% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by between 15% to 20% after the administering, compared to a TBI patient undergoing hemorrhagic shock having been administered the same amount of non-oxygen reduced conventionally stored blood. In an aspect, the cardiac output of the TBI patient undergoing hemorrhagic shock is increased by at least 2%, 5%, 10%, 15%, or 20% after the administering, compared to a TBI patient undergoing hemorrhagic shock having been administered the same amount of non-oxygen reduced conventionally stored blood.

Methods of the present disclosure provide for, and include, increasing the cardiac output of a TBI patient undergoing hemorrhagic shock to between about 1 liter per minute (L/min) and about 10 L/min, comprising administering to the patient oxygen reduced blood having an oxygen saturation (SO₂) of 20% or less prior to and during storage. In an aspect, the cardiac output of the TBI patient undergoing hemorrhagic shock is increased to between about 1 L/min and about 5 L/min after the administering. In an aspect, the cardiac output of the TBI patient undergoing hemorrhagic shock is increased to between about 1 L/min and about 8 L/min after the administering. In an aspect, the cardiac output of the TBI patient undergoing hemorrhagic shock is increased to between about 5 L/min and about 8 L/min after the administering. In an aspect, the cardiac output of the TBI patient undergoing hemorrhagic shock is increased to between about 5 L/min and about 10 L/min after the administering.

Methods of the present disclosure provide for, and include, improving hepatic function in a traumatic brain injury (TBI) patient in need thereof, comprising administering to the patient oxygen reduced blood having an oxygen saturation (SO₂) of 20% or less prior to and during storage. In aspects, improved hepatic function comprises a reduced bilirubin level, a reduced alkaline phosphatase (ALP) level, a reduced aspartate aminotransferase (AST) level, a reduced albumin plus globulin (total protein) level, a reduced interleukin 6 (IL-6) level, a reduced CXC motif chemokine ligand 1 (CXCL1) level, a reduced monocyte chemoattractant protein-1 (MCP1) level, a reduced ferritin level, an increased Glasgow Coma Scale (GCS) score, a reduced shock index (SI), and any combination thereof after the administering. In an aspect, the TBI is stabilized and the hemorrhagic shock is reversed in the patient having improved splenic function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

In an aspect, improving hepatic function in a traumatic brain injury (TBI) patient in need thereof comprises administering to the patient oxygen reduced blood having an oxygen saturation (SO₂) of 20% or less prior to and during storage, where the patient has a bilirubin level that is reduced by at least 20% after the administering, compared to the bilirubin level in a TBI patient undergoing hemorrhagic shock having been administered the same amount of non-oxygen reduced conventionally stored blood. In an aspect, the bilirubin level in the patient having a TBI accompanied by hemorrhagic shock is reduced to between about 5 millimoles per liter (mmol/L) and about 120 mmol/L after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has an alkaline phosphatase (ALP) level that is reduced by at least 20% after the administering, compared to the ALP level in a TBI patient undergoing hemorrhagic shock having been administered the same amount of non-oxygen reduced conventionally stored blood. In an aspect, the ALP level in the patient having a TBI accompanied by hemorrhagic shock is reduced to between about 30 international units per liter (IU/L) and about 200 IU/L after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has an aspartate aminotransferase (AST) level that is reduced by at least 10% after the administering, compared to the AST level in a TBI patient undergoing hemorrhagic shock having been administered the same amount of non-oxygen reduced conventionally stored blood. In an aspect, the AST level in the patient having a TBI accompanied by hemorrhagic shock is reduced to between about 5 units per liter (U/L) and about 40 U/L after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has an albumin level that is reduced after the administering, compared to the albumin level in a TBI patient undergoing hemorrhagic shock having been administered the same amount of non-oxygen reduced conventionally stored blood. In an aspect, the albumin level in the patient having a TBI accompanied by hemorrhagic shock is reduced to between about 3 grams per deciliter (g/dL) and about 6 g/dL after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has an albumin plus globulin (total protein) level that is reduced after the administering, compared to the total protein level in a TBI patient undergoing hemorrhagic shock having been administered the same amount of non-oxygen reduced conventionally stored blood. In an aspect, the albumin plus globulin (total protein) level in the patient having a TBI accompanied by hemorrhagic shock is reduced to between about 5 grams per deciliter (g/dL) and about 10 g/dL after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has an interleukin 6 (IL-6) level that is reduced by at least 20% after the administering, compared to the IL-6 level in a TBI patient undergoing hemorrhagic shock having been administered the same amount of non-oxygen reduced conventionally stored blood. In an aspect, the IL-6 level in the patient having a TBI accompanied by hemorrhagic shock is reduced to between about 3 picograms per milliliter (pg/mL) and about 8 pg/mL of blood after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has a CXC motif chemokine ligand 1 (CXCL1) level that is reduced by at least 30% after the administering, compared to the CXCL1 level in a TBI patient undergoing hemorrhagic shock having been administered the same amount of non-oxygen reduced conventionally stored blood. In an aspect, the CXCL1 level in the patient having a TBI accompanied by hemorrhagic shock is reduced to between about 30 picograms per milliliter (pg/mL) and about 90 pg/mL of serum after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has a monocyte chemoattractant protein-1 (MCP1) level of that is reduced by at least 20% after the administering, compared to the MCP1 level in a TBI patient undergoing hemorrhagic shock having been administered the same amount of non-oxygen reduced conventionally stored blood. In an aspect, the MCP1 level of the patient having a TBI accompanied by hemorrhagic shock is reduced to between about 50 picograms per milliliter (pg/mL) and about 200 pg/mL of plasma after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient has a ferritin level that is reduced by between 5% to 20% after the administering, compared to the ferritin level in a TBI patient undergoing hemorrhagic shock having been administered the same amount of non-oxygen reduced conventionally stored blood. In an aspect, the ferritin level in the TBI patient undergoing hemorrhagic shock is reduced to between about 5 milligrams per liter (mg/L) and about 260 mg/L after the administering.

In an aspect, improving hepatic function in a traumatic brain injury (TBI) patient in need thereof comprises administering to the patient oxygen reduced blood having an oxygen saturation (SO₂) of 20% or less prior to and during storage, where the patient exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having improved hepatic function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered by transfusion to the patient having a TBI accompanied by hemorrhagic shock.

Methods of the present disclosure provide for, and include, improving splenic function in a traumatic brain injury (TBI) patient in need thereof, comprising administering to the patient oxygen reduced blood having an oxygen saturation (SO₂) of 20% or less prior to and during storage. In aspects, improved splenic function comprises a reduced monocyte chemoattractant protein-1 (MCP1) level, a reduced ferritin level, an increased Glasgow Coma Scale (GCS) score, a reduced shock index (SI), and any combination thereof after the administering. In an aspect, the TBI is stabilized and the hemorrhagic shock is reversed in the patient having improved splenic function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

In an aspect, improving splenic function in a traumatic brain injury (TBI) patient in need thereof comprises administering to the patient oxygen reduced blood having an oxygen saturation (SO₂) of 20% or less prior to and during storage, where the patient has a monocyte chemoattractant protein-1 (MCP1) level that is reduced by at least 20% after the administering, compared to the MCP1 level in a TBI patient undergoing hemorrhagic shock having been administered the same amount of non-oxygen reduced conventionally stored blood. In an aspect, the MCP1 level in the patient having a TBI accompanied by hemorrhagic shock is reduced to between about 50 picograms per milliliter (pg/mL) and about 200 pg/mL after the administering.

In an aspect, improving splenic function in a traumatic brain injury (TBI) patient in need thereof comprises administering to the patient oxygen reduced blood having an oxygen saturation (SO₂) of 20% or less prior to and during storage, where the patient has a ferritin level that is reduced by between about 5% to about 20% after the administering, compared to the ferritin level in a TBI patient undergoing hemorrhagic shock having been administered the same amount of non-oxygen reduced conventionally stored blood. In an aspect, the ferritin level of the patient having a TBI accompanied by hemorrhagic shock is reduced to between about 5 milligrams per liter (mg/L) and about 260 mg/L after the administering.

In an aspect, improving splenic function in a traumatic brain injury (TBI) patient in need thereof comprises administering to the patient oxygen reduced blood having an oxygen saturation (SO₂) of 20% or less prior to and during storage, where the patient exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having improved splenic function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

Methods of the present disclosure provide for, and include, improving cardiac function in a traumatic brain injury (TBI) patient in need thereof, comprising administering to the patient oxygen reduced blood having an oxygen saturation (SO₂) of 20% or less prior to and during storage. In aspects, improved cardiac function comprises a reduced troponin level, a reduced plasma C reactive protein (CRP) level, a reduced plasma atrial natriuretic peptide (ANP) level, a reduced ferritin level, a interleukin-6 (IL-6) level, a reduced tumor necrosis factor alpha (TNF-α) level, a reduced monocyte chemoattractant protein-1 (MCP1) level, an increased cardiac output (CO), an increased Glasgow Coma Scale (GCS) score, a reduced shock index (SI), and any combination thereof after the administering. In an aspect, the TBI is stabilized and the hemorrhagic shock is reversed in the patient having improved cardiac function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

In an aspect, improving cardiac function in a traumatic brain injury (TBI) patient in need thereof comprises administering to the patient oxygen reduced blood having an oxygen saturation (SO₂) of 20% or less prior to and during storage, where the patient has a troponin level that is reduced by at least 5% after the administering, compared to the troponin level in a TBI patient undergoing hemorrhagic shock having been administered the same amount of non-oxygen reduced conventionally stored blood. In an aspect, the troponin level in the patient having a TBI accompanied by hemorrhagic shock is reduced to between about 0.01 nanograms per milliliter (ng/mL) and 0.1 ng/mL after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has a plasma C reactive protein (CRP) level that is reduced by at least 5% after the administering, compared to the CRP level in a TBI patient undergoing hemorrhagic shock having been administered the same amount of non-oxygen reduced conventionally stored blood. In an aspect, the CRP level in the patient having a TBI accompanied by hemorrhagic shock is reduced to between about 5 milligrams per liter (mg/L) and about 15 mg/L after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has a plasma atrial natriuretic peptide (ANP) level that is reduced by at least 5% after the administering, compared to the plasma ANP level in a TBI patient undergoing hemorrhagic shock having been administered the same amount of non-oxygen reduced conventionally stored blood. In an aspect, the plasma ANP level in the patient having a TBI accompanied by hemorrhagic shock is reduced to between about 5 picograms per milliliter (pg/mL) and about 30 pg/mL after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has ferritin level that is reduced by between 5% and 20% after administering, compared to the ferritin level in a TBI patient undergoing hemorrhagic shock having been administered the same amount of non-oxygen reduced conventionally stored blood. In an aspect, the ferritin level in the patient having a TBI accompanied by hemorrhagic shock is reduced to between about 5 milligrams per liter (mg/L) and about 260 mg/L after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has an interleukin-6 (IL-6) level that is reduced by at least 20% after the administering, compared to the IL-6 level in a TBI patient undergoing hemorrhagic shock having been administered the same amount of non-oxygen reduced conventionally stored blood. In an aspect, the IL-6 level in the patient having a TBI accompanied by hemorrhagic shock is reduced to between about 3 picograms per milliliter (pg/mL) and about 8 pg/mL of blood after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has a tumor necrosis factor alpha (TNF-α) level that is reduced by at least 5% after the administering, compared to the TNF-α level in a TBI patient undergoing hemorrhagic shock having been administered the same amount of non-oxygen reduced conventionally stored blood. In an aspect, the TNF-α level in the patient having a TBI accompanied by hemorrhagic shock is reduced to between about 50 picograms per milliliter (pg/mL) and about 100 pg/mL of blood after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has a monocyte chemoattractant protein-1 (MCP1) level that is reduced by at least 20% after the administering, compared to the MCP1 level in a TBI patient undergoing hemorrhagic shock having been administered the same amount of non-oxygen reduced conventionally stored blood. In an aspect, the MCP1 level in the patient having a TBI accompanied by hemorrhagic shock is reduced to between about 50 picograms per milliliter (pg/mL) and about 200 pg/mL of plasma after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient has a cardiac output (CO) that is increased by at least 5% after the administering, compared to the cardiac output of a TBI patient undergoing hemorrhagic shock having been administered the same amount of non-oxygen reduced conventionally stored blood. In an aspect, the cardiac output of the TBI patient undergoing hemorrhagic shock is increased to between about 1 liter per minute (L/min) and about 10 L/min after the administering.

In an aspect, improving cardiac function in a traumatic brain injury (TBI) patient in need thereof comprises administering to the patient oxygen reduced blood having an oxygen saturation (SO₂) of 20% or less prior to and during storage, where the patient exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having improved cardiac function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to a patient having a TBI accompanied by hemorrhagic shock by transfusion.

Methods of the present disclosure provide for, and include, improving pulmonary function in a traumatic brain injury (TBI) patient in need thereof, comprising administering to the patient oxygen reduced blood having an oxygen saturation (SO₂) of 20% or less prior to and during storage. In aspects, improved pulmonary function comprises a reduced monocyte chemoattractant protein-1 (MCP1) level, a reduced lung neutrophil content, an increased Glasgow Coma Scale (GCS) score, a reduced shock index (SI), and any combination thereof after the administering. In an aspect, the TBI is stabilized and the hemorrhagic shock is reversed in the patient having improved pulmonary function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

In an aspect, improving pulmonary function in a traumatic brain injury (TBI) patient in need thereof comprises administering to the patient oxygen reduced blood having an oxygen saturation (SO₂) of 20% or less prior to and during storage, where a myeloperoxidase (MPO) level in the patient is reduced after the administering, compared to the MPO level in a TBI patient undergoing hemorrhagic shock having been administered the same amount of non-oxygen reduced conventionally stored blood. In an aspect, the MPO level in the patient having a TBI accompanied by hemorrhagic shock is reduced to between about 10 nanograms per milliliter (ng/mL) and about 240 ng/mL after the administering.

In an aspect, improving pulmonary function in a traumatic brain injury (TBI) patient in need thereof comprises administering to the patient oxygen reduced blood having an oxygen saturation (SO₂) of 20% or less prior to and during storage, where the patient has a lung neutrophil content that is reduced after the administering, compared to the lung neutrophil content of a TBI patient undergoing hemorrhagic shock having been administered the same amount of non-oxygen reduced conventionally stored blood. In an aspect, the lung neutrophil content of the patient having a TBI accompanied by hemorrhagic shock is reduced to between about 40% and about 70% after the administering. In an aspect, the lung neutrophil content of the patient having a TBI accompanied by hemorrhagic shock is measured by flow cytometry.

In an aspect, improving pulmonary function in a traumatic brain injury (TBI) patient in need thereof comprises administering to the patient oxygen reduced blood having an oxygen saturation (SO₂) of 20% or less prior to and during storage, where the patient exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having improved pulmonary function after the administering. In an aspect, oxygen reduced blood having an oxygen saturation (SO₂) of 10% or less prior to and during storage is administered to a patient having a TBI accompanied by hemorrhagic shock. In an aspect, oxygen reduced blood is administered to a patient having a TBI accompanied by hemorrhagic shock by transfusion.

Methods of the present disclosure provide for, and include, improving renal function in a traumatic brain injury (TBI) patient in need thereof, comprising administering to the patient oxygen reduced blood having an oxygen saturation (SO₂) of 20% or less prior to and during storage. In aspects, improved renal function comprises a reduced urine creatinine level, an increased Glasgow Coma Scale (GCS) score, a reduced shock index (SI), and any combination thereof after the administering. In an aspect, the TBI is stabilized and the hemorrhagic shock is reversed in the patient having improved renal function after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

In an aspect, improving renal function in a traumatic brain injury (TBI) patient in need thereof comprises administering to the patient oxygen reduced blood having an oxygen saturation (SO₂) of 20% or less prior to and during storage, where the urine creatinine level in the patient is reduced by at least 5% after the administering, compared to the urine creatinine level in a TBI patient undergoing hemorrhagic shock having been administered the same amount of non-oxygen reduced conventionally stored blood. In an aspect, the urine creatinine level in the patient having a TBI accompanied by hemorrhagic shock is reduced to between about 10 milligrams per deciliter (mg/dL) and about 350 mg/dL after the administering.

In an aspect, improving renal function in a traumatic brain injury (TBI) patient in need thereof comprises administering to the patient oxygen reduced blood having an oxygen saturation (SO₂) of 20% or less prior to and during storage, where the patient exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having improved renal function after the administering. In an aspect, oxygen reduced blood having an oxygen saturation (SO₂) of 10% or less prior to and during storage is administered to a patient having a TBI accompanied by hemorrhagic shock. In an aspect, oxygen reduced blood is administered to the patient having a TBI accompanied by hemorrhagic shock by transfusion.

Methods of the present disclosure provide for, and include, increasing the level of a plasma serum cytokine selected from the group consisting of interleukin 6 (IL-6), CXC motif chemokine ligand 1 (CXCL1), interleukin 10 (IL-10), and any combination thereof, in a traumatic brain injury (TBI) patient in need thereof, comprising administering to the patient oxygen reduced blood having an oxygen saturation (SO₂) of 20% or less prior to and during storage. In an aspect, the plasma level of IL-10 is increased in the patient by at least 5% after the administering, compared to the level of IL-10 in a TBI patient undergoing hemorrhagic shock having been administered the same amount of non-oxygen reduced conventionally stored blood. In an aspect, the plasma level of IL-10 is increased to between about 3 picograms per milliliter (pg/mL) and about 12 pg/mL after the administering. In an aspect, the patient having a TBI accompanied by hemorrhagic shock exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having an increased level of a plasma serum cytokine after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to a patient having a TBI accompanied by hemorrhagic shock by transfusion.

Methods of the present disclosure provide for, and include, modulating a stress level marker selected from the group consisting of epinephrine, norepinephrine, cortisol, and any combination thereof, in a traumatic brain injury (TBI) patient in need thereof, comprising administering to the patient oxygen reduced blood having an oxygen saturation (SO₂) of 20% or less prior to and during storage. In an aspect, the stress level marker is a plasma stress level marker. In an aspect, the plasma level of epinephrine is reduced by at least 5% in the patient after the administering, compared to the plasma level of epinephrine in a TBI patient undergoing hemorrhagic shock having been administered the same amount of non-oxygen reduced conventionally stored blood. In an aspect, the plasma level of epinephrine in the TBI patient undergoing hemorrhagic shock is reduced to between about 5 picograms per milliliter (pg/mL) and about 150 pg/mL after the administering. In an aspect, the plasma level of epinephrine is reduced by at least 5% in the patient after the administering, compared to the plasma level of epinephrine in a TBI patient undergoing hemorrhagic shock having been administered the same amount of non-oxygen reduced conventionally stored blood. In an aspect, the plasma level of epinephrine in the TBI patient undergoing hemorrhagic shock is reduced to between about 50 picograms per milliliter (pg/mL) and about 2000 pg/mL after the administering. In an aspect, the plasma level of cortisol is reduced by at least 5% in the patient after the administering, compared to the plasma level of cortisol in a TBI patient undergoing hemorrhagic shock having been administered the same amount of non-oxygen reduced conventionally stored blood. In an aspect, the plasma level of epinephrine in the TBI patient undergoing hemorrhagic shock is reduced to between about 2 milligrams per deciliter (mg/dL) and about 25 mg/dL after the administering. In an aspect, the patient having a TBI accompanied by hemorrhagic shock exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having a modulated stress marker after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to a patient having a TBI accompanied by hemorrhagic shock by transfusion.

Methods of the present disclosure provide for, and include, increasing tissue oxygenation (oxygen perfusion) in one or more vital organs selected from the group consisting of the kidney, the liver, the lungs, the spleen, the heart, and any combination thereof in a traumatic brain injury (TBI) patient in need thereof, comprising administering to the patient oxygen reduced blood having an oxygen saturation (SO₂) of 20% or less prior to and during storage. In an aspect, the tissue oxygen saturation in each of the one or more vital organs is increased by at least 5% after the administering. In an aspect, the tissue oxygen saturation in each of the one or more vital organs is increased by at least 10% after the administering. In an aspect, the tissue oxygen saturation in each of the one or more vital organs is increased by at least 15% after the administering. In an aspect, the tissue oxygen saturation in each of the one or more vital organs is increased by at least 25% after the administering. In an aspect, the tissue oxygen saturation in each of the one or more vital organs is increased by between 10% and 20% after the administering. In an aspect, the tissue oxygen saturation in each of the one or more vital organs is increased by between 5% and 25% after the administering. In an aspect, the tissue oxygen saturation in the patient having a TBI accompanied by hemorrhagic shock is measured by administering an effective amount of pimonidazole to the patient and detecting the distribution of the pimonidazole hydrochloride in the one or more vital organs of the patient. In an aspect, the patient having a TBI accompanied by hemorrhagic shock exhibits a Glasgow Coma Scale (GCS) score of 12 or less prior to the administering. In an aspect, the patient having a TBI accompanied by hemorrhagic shock exhibits a GCS score of between 3 and 12 prior to the administering. In an aspect, the patient having a TBI accompanied by hemorrhagic shock has a GCS score of between 13 and 15 after the administering. In an aspect, the patient having a TBI accompanied by hemorrhagic shock has a shock index (SI) of greater than 0.9 prior to the administering. In an aspect, the SI of the patient having a TBI accompanied by hemorrhagic shock is reduced to between 0.5 and 0.9 after the administering. In an aspect, the TBI is stabilized and the hemorrhagic shock is reversed in the patient having increased tissue oxygenation in one or more vital organs after the administering. In an aspect, oxygen reduced blood having an oxygen saturation (SO₂) of 10% or less prior to and during storage is administered to the patient having a TBI accompanied by hemorrhagic shock. In an aspect, oxygen reduced blood is administered to the patient having a TBI accompanied by hemorrhagic shock by transfusion.

As used herein, the singular forms “a,” “an,” and “the” include plural references unless the context clearly dictates otherwise. For example, the term “a compound” or “at least one compound” can include a plurality of compounds, including mixtures thereof.

As used herein the term “about” refers to =10%.

As used herein, the term “adverse event” includes an event resulting from a traumatic brain injury (TBI), hemorrhagic shock, or a combination of TBI and hemorrhagic shock in a TBI patient undergoing hemorrhagic shock.

As used herein, the term “blood” refers to whole blood, leukoreduced red blood cells (RBCs), platelet reduced RBCs, and leukocyte and platelet reduced RBCs. The term “blood” further includes packed red blood cells, platelet reduced packed red blood cells, leukocyte reduced packed red blood cells, and leukocyte and platelet reduced packed red blood cells. The temperature of blood can vary depending on the stage of the collection process, starting at the normal body temperature of 37 °C at the time and point of collection, but decreasing rapidly to about 30 °C as soon as the blood leaves the patient's body and further thereafter to room temperature in about 6 hours when untreated, and ultimately being refrigerated at between about 4 °C and 6 °C. Human red blood cells in vivo are in a dynamic state. The red blood cells contain hemoglobin, the iron-containing protein that carries oxygen throughout the body and gives red blood its color. The percentage of blood volume composed of red blood cells is called the hematocrit. As used herein, unless otherwise limited, RBCs also includes packed red blood cells (pRBCs). Packed red blood cells are prepared from whole blood using centrifugation techniques commonly known in the art. As used herein, unless otherwise indicated, the hematocrit of pRBCs is about 70%. As used herein, oxygen reduced stored RBCs can include oxygen and carbon dioxide reduced stored RBCs. As used herein, oxygen reduced (OR) blood can include oxygen and carbon dioxide (OCR) reduced blood.

As used herein, the terms “comprises,” “comprising,” “includes,” “including,” “having,” and their conjugates mean “including but not limited to.”

As used herein, the term “consisting of” means “including and limited to.”

As used herein, the term “consisting essentially of” means that the composition, method or structure can include additional ingredients, steps and/or parts, but only if the additional ingredients, steps and/or parts do not materially alter the basic and novel characteristics of the claimed composition, method or structure.

As used herein, “Glasgow Coma Scale” (also abbreviated herein as “GCS”) refers to a scoring system to objectively describe the extent of impaired consciousness in any and all types of acute medical and trauma patients. The scale assesses patients according to three aspects of responsiveness: eye-opening response, motor response, and verbal response. A patient's responsiveness in each aspect is given a score ranging from 1 (indicating no response) to 4, 5, or 6 (indicating the normal values for eye-opening response, motor response, and verbal response, respectively). See Mehta et al., “Glasgow coma scale explained,” The BMJ, 2019; 365:|1296 (2019). The GCS score for a patient as used herein is the sum of the patient's scores in each of the abovementioned aspects of responsiveness.

As used herein, “shock index” (also abbreviated herein as “SI”) refers to a formula for assessing the severity of hypovolemic (including hemorrhagic) shock in a patient. The formula is the heart rate (HR) of a patient divided by the systolic blood pressure (SBP) of the patient. In general, a patient with an SI equal to or greater than 0.9 is at higher risk for adverse outcomes, including mortality.

As used herein, the term “hemorrhagic shock” is shock brought on by a loss of circulating blood volume and/or oxygen carrying capacity. Hemorrhagic shock results from any condition associated with blood loss, internal (e.g., gastrointestinal bleeding) or external hemorrhage, and trauma (e.g., penetrating or blunt trauma), among others. Hemorrhagic shock may be considered a type of hypovolemic shock specifically related to blood loss.

As used herein, “hypovolemic shock” is shock brought on by a loss of fluids (including blood or extracellular fluids). Hypovolemic shock results in organ dysfunction due to inadequate tissue perfusion. Hypovolemic shock results from any condition associated with blood and/or fluid loss, such as internal or external hemorrhage, trauma, surgery, prolonged dehydration, diarrhea, and severe burns, among others.

As used herein, “a patient having a traumatic brain injury accompanied by hemorrhagic shock” and a “traumatic brain injury (TBI) patient undergoing hemorrhagic shock” are used interchangeably and should be understood to describe a patient having both a traumatic brain injury and hemorrhagic shock. For example, “a traumatic brain injury accompanied by hemorrhagic shock” is meant to describe the same condition afflicting “a TBI patient undergoing hemorrhagic shock.” In a patient presenting with both a traumatic brain injury and hemorrhagic shock, the traumatic brain injury may induce the hemorrhagic shock in the patient. As used herein, “traumatic brain injury accompanied by hemorrhagic shock” is also abbreviated as “TBI+HS.”

As used herein, the terms “higher”, “greater” or “increased” means that the measured values of oxygen reduced blood, when compared to the measured values of otherwise equivalently treated non-oxygen reduced conventionally stored blood, are at least 1 standard deviation greater, with a sample size of at least 2 for each compared measured condition.

As used herein, the terms “injury”, “damage”, and “failure” refer to an organ not functioning properly or not functioning as is expected in a person or animal without disease or injury.

As used herein, the term “less than” refers to a smaller amount and an amount greater than zero.

As used herein, the terms “patient” and “subject” are used interchangeably to mean a human or animal in need of treatment with the methods disclosed herein.

As used herein, the terms “reduce”, “reduced”, “lower”, “decreased” or “less” means that the measured values of oxygen reduced blood when compared to the measured values of otherwise equivalently treated non-oxygen reduced conventionally stored blood, are at least 1 standard deviation lower, with a sample size of at least 2 for each compared measured condition.

Various aspects of this disclosure may be presented as a fold change (e.g., a fold increase or a fold reduction). Within the context of measured values of oxygen reduced blood when compared to the measured values of otherwise equivalently treated non-oxygen reduced conventionally stored blood in the disclosure, the word “fold” should be understood to entail a multiplication in the case of a fold increase, or a division in the case of a fold reduction. For example, “increased by 2 fold” means a multiplication by 2. As another example, “reduced by 2 fold” means a division by 2.

As used herein, the term “traumatic brain injury” (also abbreviated herein as “TBI”) is a form of acquired brain injury that occurs when a sudden trauma to the head causes damage to the brain. Traumatic brain injury may result when the head suddenly and violently hits an object, or when an object pierces the skull and enters brain tissue.

As used herein, “stabilizes”, “stabilized”, and its conjugates refer to a patient with a traumatic brain injury or a traumatic brain injury accompanied by hemorrhagic shock who is no longer at risk for further injury stemming from the traumatic brain injury (also known as secondary brain injury). Procedures to stabilize a patient with a traumatic brain injury or a traumatic brain injury accompanied by hemorrhagic shock are known in the art and include, but are not limited to, ensuring proper oxygen supply to the brain and the body, maintaining adequate blood flow, controlling blood pressure, and any combination thereof. Ensuring proper oxygen supply to the brain and the body may involve, for example, increasing the oxygen saturation level of the patient to between about 90% and about 98%. Maintaining adequate blood flow may involve, for example, increasing the cardiac output of the patient to between about 2 liter per minute (L/min) to about 10 L/min. Controlling blood pressure may involve, for example, increasing the systolic blood pressure of the patient to equal to or greater than 100 millimeters of mercury (mmHg). In some aspects, the systolic blood pressure of the patient may be increased to equal to or greater than 110 mmHg.

As used herein, a “unit” of blood is about 450-500 mL including anticoagulant. Suitable anticoagulants include CPD, CPDA1, ACD, and ACD-A.

As used herein, the terms “administer”, “administration”, and “administering” refer to the delivery of blood to a patient, e.g., by transfusion.

Throughout this application, various aspects of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. Description of ranges may include terms such as “from” and “between.” For example, description of a range such as “from 1 to 6” should be considered to have specifically disclosed subranges such as “from 1 to 3,” “from 1 to 4,” “from 1 to 5,” “from 2 to 4,” “from 2 to 6,” “from 3 to 6,” etc., as well as individual numbers within that range, for example, 1, 2, 3, 4, 5, and 6. This applies regardless of the breadth of the range. As another example, description of a range such as “between 1 and 6” should be considered to have specifically disclosed subranges such as “between 1 and 3,” “between 1 and 4,” “between 1 and 5,” “between 2 and 4,” “between 2 and 6,” “between 3 and 6,” etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicated number “and” a second indicated number, and “ranging/ranges from” a first indicated number “to” a second indicated number are used interchangeably herein and are meant to include the first and second indicated numbers and all the fractional and integral numerals there between. Similarly, the phrases “between” a first indicated number “and” a second indicated number, and “between” a first indicated number “to” a second indicated number are also used interchangeably herein and are meant to include the first and second indicated numbers and all the fractional and integral numerals there between.

As used herein, the term “method” refers to manners, means, techniques, and procedures for accomplishing a given task including, but not limited to, administering to a human patient in need of treatment of a traumatic brain injury accompanied by hemorrhagic shock oxygen reduced stored blood having an initial oxygen saturation of 20% or less prior to and during storage.

### EMBODIMENTS

Embodiment 1. A method of treating a traumatic brain injury (TBI) accompanied by hemorrhagic shock in a patient in need thereof comprising administering oxygen reduced blood having an oxygen saturation (SO₂) of 20% or less prior to and during storage to a TBI patient undergoing hemorrhagic shock, wherein the patient exhibits a Glasgow Coma Scale (GCS) score of 12 or less prior to the administering, and the patient has a shock index (SI) of greater than 0.9 prior to the administering, wherein the patient exhibits a GCS score of at least 13 after the administering, and wherein the hemorrhagic shock is reversed after the administering.

Embodiment 2. The method of Embodiment 1, wherein the Glasgow Coma Scale (GCS) score of the patient is between 3 and 12 prior to the administering.

Embodiment 3. The method of Embodiment 1 or Embodiment 2, wherein the Glasgow Coma Scale (GCS) score of the patient is between 13 and 15 after the administering.

Embodiment 4. The method of any one of Embodiments 1 to 3, wherein the shock index (SI) of the patient is reduced to between 0.5 and 0.9 after the administering.

Embodiment 5. The method of any one of Embodiments 1 to 4, wherein an arterial oxygen saturation (SO₂) of the patient is increased by at least 10% after the administering, compared to a TBI patient undergoing hemorrhagic shock having been administered the same amount of non-oxygen reduced conventionally stored blood.

Embodiment 6. The method of Embodiment 5, wherein the arterial SO₂ is increased by at least 20% after the administering, compared to a TBI patient undergoing hemorrhagic shock having been administered the same amount of non-oxygen reduced conventionally stored blood.

Embodiment 7. The method of any one of Embodiments 1 to 6, wherein a venous oxygen saturation (SO₂) of the patient is increased by at least 10% after the administering, compared to a TBI patient undergoing hemorrhagic shock having been administered the same amount of non-oxygen reduced conventionally stored blood.

Embodiment 8. The method of Embodiment 7, wherein the venous SO₂ is increased by at least 20% after the administering, compared to a TBI patient undergoing hemorrhagic shock having been administered the same amount of non-oxygen reduced conventionally stored blood.

Embodiment 9. The method of any one of Embodiments 1 to 8, wherein the patient has improved pulmonary function after the administering.

Embodiment 10. The method of Embodiment 9, wherein the patient has reduced levels of CXC motif chemokine ligand 1 (CXCL1), myeloperoxidase (MPO), lung neutrophils, or any combination thereof after the administering.

Embodiment 11. The method of Embodiment 9 or Embodiment 10, wherein the patient has a reduced level of myeloperoxidase (MPO) after the administering, compared to a TBI patient undergoing hemorrhagic shock having been administered the same amount of non-oxygen reduced conventionally stored blood.

Embodiment 12. The method of Embodiment 11, wherein the reduced level of MPO level is between 10 nanograms per milliliter (ng/mL) and 240 ng/mL after the administering.

Embodiment 13. The method of any one of Embodiments 9 to 12, wherein the patient has a reduced lung neutrophil content after the administering, compared to a TBI patient undergoing hemorrhagic shock having been administered the same amount of non-oxygen reduced conventionally stored blood.

Embodiment 14. The method of Embodiment 13, wherein the lung neutrophil content is reduced to between 40% and 70% after the administering.

Embodiment 15. The method of Embodiment 13 or Embodiment 14, wherein the lung neutrophil content is measured by flow cytometry.

Embodiment 16. The method of any one of Embodiments 1 to 8, wherein the patient has improved hepatic function after the administering.

Embodiment 17. The method of Embodiment 16, wherein the patient has reduced levels of alanine transaminase (ALT), aspartate transaminase (AST), alkaline phosphatase (ALP), bilirubin, interleukin 6 (IL-6), CXC motif chemokine ligand 1 (CXCL1), monocyte chemoattractant protein-1 (MCP1), ferritin, albumin, albumin plus globulin (total protein), or any combination thereof after the administering.

Embodiment 18. The method of Embodiment 16 or Embodiment 17, wherein the patient has an alkaline phosphatase (ALP) level that is reduced by at least 20% after the administering, compared to a TBI patient undergoing hemorrhagic shock having been administered the same amount of non-oxygen reduced conventionally stored blood.

Embodiment 19. The method of Embodiment 18, wherein the ALP level is reduced to between 30 international units per liter (IU/L) and 200 IU/L after the administering.

Embodiment 20. The method of any one of Embodiments 16 to 19, wherein the patient has an aspartate aminotransferase (AST) level that is reduced by at least 10% after the administering, compared to a TBI patient undergoing hemorrhagic shock having been administered the same amount of non-oxygen reduced conventionally stored blood.

Embodiment 21. The method of Embodiment 20, wherein the AST level is reduced to between 5 units per liter (U/L) and 40 U/L after the administering.

Embodiment 22. The method of any one of Embodiments 16 to 21, wherein the patient has an albumin level that is reduced after the administering, compared to a TBI patient undergoing hemorrhagic shock having been administered the same amount of non-oxygen reduced conventionally stored blood.

Embodiment 23. The method of Embodiment 22, wherein the albumin level is reduced to between 3 grams per deciliter (g/dL) and 6 g/dL after the administering.

Embodiment 24. The method of any one of Embodiments 16 to 23, wherein the patient has an albumin plus globulin (total protein) level that is reduced after the administering, compared to a TBI patient undergoing hemorrhagic shock having been administered the same amount of non-oxygen reduced conventionally stored blood.

Embodiment 25. The method of Embodiment 24, wherein the albumin plus globulin (total protein) level is reduced to between 5 grams per deciliter (g/dL) and 10 g/dL after the administering.

Embodiment 26. The method of any one of Embodiments 16 to 25, wherein the patient has a bilirubin level that is reduced by at least 20% after the administering, compared to a TBI patient undergoing hemorrhagic shock having been administered the same amount of non-oxygen reduced conventionally stored blood.

Embodiment 27. The method of Embodiment 26, wherein the bilirubin level is reduced to between 5 millimoles per liter (mmol/L) and 120 mmol/L after the administering.

Embodiment 28. The method of any one of Embodiments 16 to 27, wherein the patient has an interleukin 6 (IL-6) level that is reduced by at least 20% after the administering, compared to a TBI patient undergoing hemorrhagic shock having been administered the same amount of non-oxygen reduced conventionally stored blood.

Embodiment 29. The method of Embodiment 28, wherein the IL-6 level is reduced to between 3 picograms per milliliter (pg/mL) and 8 pg/mL of blood after the administering. Embodiment 30. The method of any one of Embodiments 16 to 29, wherein the patient has a CXC motif chemokine ligand 1 (CXCL1) level that is reduced by at least 30% after the administering, compared to a TBI patient undergoing hemorrhagic shock having been administered the same amount of non-oxygen reduced conventionally stored blood.

Embodiment 31. The method of Embodiment 30, wherein the CXCL1 level is reduced to between 30 picograms per milliliter (pg/mL) and 90 pg/mL of serum after the administering.

Embodiment 32. The method of any one of Embodiments 16 to 31, wherein the patient has a monocyte chemoattractant protein-1 (MCP1) level that is reduced by at least 20% after the administering, compared to a TBI patient undergoing hemorrhagic shock having been administered the same amount of non-oxygen reduced conventionally stored blood.

Embodiment 33. The method of Embodiment 32, wherein the MCP1 level is reduced to between 50 picograms per milliliter (pg/mL) and 200 pg/mL of plasma after the administering.

Embodiment 34. The method of any one of Embodiments 16 to 33, wherein the patient has a ferritin level that is reduced by between 5% to 20% after the administering, compared to a TBI patient undergoing hemorrhagic shock having been administered the same amount of non-oxygen reduced conventionally stored blood.

Embodiment 35. The method of Embodiment 34, wherein the ferritin level is reduced to between 5 milligrams per milliliter (mg/L) and 260 mg/L after the administering.

Embodiment 36. The method of any one of Embodiments 1 to 8, wherein the patient has improved splenic function after the administering.

Embodiment 37. The method of Embodiment 36, wherein the patient has reduced levels of interleukin 6 (IL-6), CXC motif chemokine ligand 1 (CXCL1), monocyte chemoattractant protein-1 (MCP1), ferritin, or any combination thereof after the administering.

Embodiment 38. The method of Embodiment 36 or Embodiment 37, wherein the patient has a monocyte chemoattractant protein-1 (MCP1) level that is reduced by at least 20% after the administering, compared to a TBI patient undergoing hemorrhagic shock having been administered the same amount of non-oxygen reduced conventionally stored blood.

Embodiment 39. The method of Embodiment 38, wherein the MCP1 level is reduced to between 50 picograms per milliliter (pg/mL) and 200 pg/mL of plasma after the administering.

Embodiment 40. The method of any one of Embodiments 36 to 39, wherein the patient has a ferritin level that is reduced by between 5% and 20% after the administering, compared to a TBI patient undergoing hemorrhagic shock having been administered the same amount of non-oxygen reduced conventionally stored blood.

Embodiment 41. The method of Embodiment 40, wherein the ferritin level is reduced to between 5 milligrams per liter (mg/L) and 260 mg/L after the administering. Embodiment 42. The method of any one of Embodiments 1 to 8, wherein the patient has improved cardiac function after the administering.

Embodiment 43. The method of Embodiment 42, wherein the patient has reduced levels of interleukin 6 (IL-6), tumor necrosis factor alpha (TNF alpha), monocyte chemoattractant protein-1 (MCP1), troponin, plasma C reactive protein (CRP), plasma atrial natriuretic peptide (ANP), ferritin, or any combination thereof after the administering.

Embodiment 44. The method of Embodiment 42 or Embodiment 43, wherein the patient has one or more improved cardiac parameters after the administering, wherein the one or more improved cardiac parameters are selected from the group consisting of an increased stroke volume (SV), an increased stroke work (SW), an increased cardiac output (CO), an increased contractility, an increased arterial elastance, an increased internal energy utilization (IEU), a reduced systolic volume (Ves), an increased end diastolic volume (Ved), a reduced systemic vascular resistance (SVR), and any combination thereof.

Embodiment 45. The method of any one of Embodiments 42 to 44, wherein the patient has a troponin level that is reduced by at least 5% after the administering, compared to a TBI patient undergoing hemorrhagic shock having been administered the same amount of non-oxygen reduced conventionally stored blood.

Embodiment 46. The method of Embodiment 45, wherein the troponin level is reduced to between 0.01 nanograms per milliliter (ng/mL) and 0.1 ng/mL after the administering.

Embodiment 47. The method of any one of Embodiments 42 to 46, wherein the patient has a plasma C reactive protein (CRP) level that is reduced by at least at least 5% after the administering, compared to a TBI patient undergoing hemorrhagic shock having been administered the same amount of non-oxygen reduced conventionally stored blood.

Embodiment 48. The method of Embodiment 47, wherein the plasma CRP level is reduced to between 5 milligrams per liter (mg/L) and 15 mg/L after the administering.

Embodiment 49. The method of any one of Embodiments 42 to 48, wherein the patient has a plasma atrial natriuretic peptide (ANP) level that is reduced by at least 5% after the administering, compared to a TBI patient undergoing hemorrhagic shock having been administered the same amount of non-oxygen reduced conventionally stored blood.

Embodiment 50. The method of Embodiment 49, wherein the plasma ANP level is reduced to between 5 picograms per milliliter (pg/mL) and 30 pg/mL after the administering.

Embodiment 51. The method of any one of Embodiments 42 to 50, wherein the patient has a ferritin level that is reduced by between 5% and 20% after the administering, compared to a TBI patient undergoing hemorrhagic shock having been administered the same amount of non-oxygen reduced conventionally stored blood.

Embodiment 52. The method of Embodiment 51, wherein the ferritin level is reduced to between 5 picograms per liter (mg/L) and 260 mg/L after the administering.

Embodiment 53. The method of any one of Embodiments 42 to 52, wherein the patient has an interleukin-6 (IL-6) level that is reduced by at least 20% after the administering, compared to a TBI patient undergoing hemorrhagic shock having been administered the same amount of non-oxygen reduced conventionally stored blood.

Embodiment 54. The method of Embodiment 53, wherein the IL-6 level is reduced to between 3 picograms per milliliter (pg/mL) and 8 pg/mL of blood after administering.

Embodiment 55. The method of any one of Embodiments 42 to 54, wherein the patient has a tumor necrosis factor alpha (TNF-α) level that is reduced by at least 5% after the administering, compared to a TBI patient undergoing hemorrhagic shock having been administered the same amount of non-oxygen reduced conventionally stored blood.

Embodiment 56. The method of Embodiment 55, wherein the TNF-α level is reduced to between 50 picograms per milliliter (pg/mL) and 100 pg/mL after the administering.

Embodiment 57. The method of any one of Embodiments 42 to 56, wherein the patient has a monocyte chemoattractant protein-1 (MCP1) level that is reduced by at least 20% after the administering, compared to a TBI patient undergoing hemorrhagic shock having been administered the same amount of non-oxygen reduced conventionally stored blood.

Embodiment 58. The method of Embodiment 57, wherein the MCP1 level is reduced to between 50 picograms per milliliter (pg/mL) and 200 pg/mL of plasma after the administering.

Embodiment 59. The method of any one of Embodiments 1 to 58, wherein the patient has modified levels of plasma serum cytokines after the administering, wherein the plasma serum cytokines are selected from the group consisting of interleukin 6 (IL-6), CXC motif chemokine ligand 1 (CXCL1), interleukin 10 (IL-10), and any combination thereof.

Embodiment 60. The method of any one of Embodiments 1 to 59, wherein the patient has modified levels of stress markers after the administering, wherein the stress markers are selected from the group consisting of epinephrine, norepinephrine, cortisol, and any combination thereof.

Embodiment 61. The method of any one of Embodiments 1 to 60, wherein the patient has a plasma serum interleukin 10 (IL-10) level that is increased by at least 5% after the administering, compared to a TBI patient undergoing hemorrhagic shock having been administered the same amount of non-oxygen reduced conventionally stored blood.

Embodiment 62. The method of Embodiment 61, wherein the plasma serum IL-10 level is increased to between 3 picograms per milliliter (pg/mL) and 12 pg/mL after the administering.

Embodiment 63. The method of any one of Embodiments 1 to 62, wherein the patient has an epinephrine level that is reduced by at least 5% after the administering, compared to a TBI patient undergoing hemorrhagic shock having been administered the same amount of non-oxygen reduced conventionally stored blood.

Embodiment 64. The method of Embodiment 63, wherein the epinephrine level is reduced to between 5 picograms per milliliter (pg/mL) and 150 pg/mL after the administering.

Embodiment 65. The method of any one of Embodiments 1 to 64, wherein the patient has a norepinephrine level that is reduced by at least 5% after the administering, compared to a TBI patient undergoing hemorrhagic shock having been administered the same amount of non-oxygen reduced conventionally stored blood.

Embodiment 66. The method of Embodiment 65, wherein the norepinephrine level is reduced to between 50 picograms per milliliter (pg/mL) and 2000 pg/mL after the administering.

Embodiment 67. The method of any one of Embodiments 1 to 66, wherein the patient has a cortisol level that is reduced by at least 5% after the administering, compared to a TBI patient undergoing hemorrhagic shock having been administered the same amount of non-oxygen reduced conventionally stored blood.

Embodiment 68. The method of Embodiment 67, wherein the cortisol level is reduced to between 2 milligrams per deciliter (mg/dL) and 25 mg/dL after the administering.

Embodiment 69. The method of any one of Embodiments 1 to 8, wherein the patient has improved renal function after the administering.

Embodiment 70. The method of Embodiment 69, wherein the patient has reduced levels of neutrophil gelatinase-associated lipocalin (NGAL), urine creatinine, serum creatinine, blood urea nitrogen (BUN), or any combination thereof after the administering.

Embodiment 71. The method of Embodiment 69 or Embodiment 70, wherein the patient has a urine creatinine level that is reduced by at least 5% after the administering, compared to a TBI patient undergoing hemorrhagic shock having been administered the same amount of non-oxygen reduced conventionally stored blood.

Embodiment 72. The method of Embodiment 71, wherein the urine creatinine level is reduced to between 10 milligrams per deciliter (mg/dL) and 350 mg/dL after the administering.

Embodiment 73. The method of any one of Embodiments 1 to 72, wherein the patient has increased tissue oxygen saturation in one or more vital organs after the administering, wherein the one or more vital organs are selected from the group consisting of the kidney, the liver, the lungs, the spleen, the heart, and any combination thereof.

Embodiment 74. The method of Embodiment 73, wherein the tissue oxygen saturation in each of the one or more vital organs is increased by at least 10%.

Embodiment 75. The method of Embodiment 74, wherein the tissue oxygen saturation in each of the one or more vital organs is increased by at least 25%.

Embodiment 76. The method of any one of Embodiments 73 to 75, wherein the tissue oxygen saturation is measured by administering an effective amount of pimonidazole to the patient and detecting the distribution of the pimonidazole in the one or more vital organs of the patient.

Embodiment 77. The method of any one of Embodiments 1 to 76, wherein the patient has a blood lactate level that is greater than 3.0 millimoles per liter (mmol/L) prior to the administering.

Embodiment 78. The method of Embodiment 77, wherein the blood lactate level is reduced by at least 15% after the administering, compared to a TBI patient undergoing hemorrhagic shock having been administered the same amount of non-oxygen reduced conventionally stored blood.

Embodiment 79. The method of Embodiment 77 or Embodiment 78, wherein the blood lactate level is reduced to between 0.5 millimoles per liter (mmol/L) and 3.0 mmol/L after the administering.

Embodiment 80. The method of any one of Embodiments 1 to 79, wherein the patient has a blood glucose level that is greater than 120 milligrams per deciliter (mg/dL) prior to the administering.

Embodiment 81. The method of Embodiment 80, wherein the blood glucose level is reduced by at least 5% after the administering, compared to a TBI patient undergoing hemorrhagic shock having been administered the same amount of non-oxygen reduced conventionally stored blood.

Embodiment 82. The method of Embodiment 80 or Embodiment 81, wherein the blood glucose level is reduced to between 70 milligrams per deciliter (mg/dL) and 120 mg/dL after the administering.

Embodiment 83. The method of any one of Embodiments 1 to 82, wherein the patient has a hematocrit of less than 35% prior to the administering.

Embodiment 84. The method of Embodiment 83, wherein the hematocrit is increased by at least 10% after the administering, compared to a TBI patient undergoing hemorrhagic shock having been administered the same amount of non-oxygen reduced conventionally stored blood.

Embodiment 85. The method of Embodiment 83 or Embodiment 84, wherein the hematocrit is increased to between 35% and 55% after the administering.

Embodiment 86. The method of any one of Embodiments 1 to 85, wherein the patient has increased levels of one or more minerals after the administering, wherein the one or more minerals are selected from the group consisting of sodium, potassium, calcium, and any combination thereof.

Embodiment 87. The method of any one of Embodiments 1 to 86, wherein the oxygen reduced blood has an oxygen saturation (SO₂) of 10% or less prior to and during storage.

Embodiment 88. The method of any one of Embodiments 1 to 87, wherein the administering is by transfusion.

Embodiment 89. A method of improving hepatic function in a traumatic brain injury (TBI) patient in need thereof, comprising administering oxygen reduced blood having an oxygen saturation (SO₂) of 20% or less prior to and during storage, wherein the patient has a traumatic brain injury accompanied by hemorrhagic shock, and wherein the patient has a bilirubin level that is reduced by at least 20% after the administering, compared to a TBI patient undergoing hemorrhagic shock having been administered the same amount of non-oxygen reduced conventionally stored blood.

Embodiment 90. The method of Embodiment 89, wherein the bilirubin level is reduced to between 5 millimoles per liter (mmol/L) and 120 mmol/L after the administering.

Embodiment 91. The method of Embodiment 89 or Embodiment 90, wherein the patient has an alkaline phosphatase (ALP) level of that is reduced by at least 20% after the administering, compared to a TBI patient undergoing hemorrhagic shock having been administered the same amount of non-oxygen reduced conventionally stored blood.

Embodiment 92. The method of Embodiment 91, wherein the ALP level is reduced to between 30 international units per liter (IU/L) and 200 IU/L after the administering.

Embodiment 93. The method of any one of Embodiments 89 to 92, wherein the patient has an aspartate aminotransferase (AST) level that is reduced by at least 10% after the administering, compared to a TBI patient undergoing hemorrhagic shock having been administered the same amount of non-oxygen reduced conventionally stored blood.

Embodiment 94. The method of Embodiment 93, wherein the AST level is reduced to between 5 units per liter (U/L) and 40 U/L after the administering.

Embodiment 95. The method of any one of Embodiments 89 to 94, wherein the patient has an has an albumin level that is reduced after the administering, compared to a TBI patient undergoing hemorrhagic shock having been administered the same amount of non-oxygen reduced conventionally stored blood.

Embodiment 96. The method of Embodiment 95, wherein the albumin level is reduced to between 3 grams per deciliter (g/dL) and 6 g/dL after the administering.

Embodiment 97. The method of any one of Embodiments 89 to 96, wherein the patient has an has an albumin plus globulin (total protein) level that is reduced after the administering, compared to a TBI patient undergoing hemorrhagic shock having been administered the same amount of non-oxygen reduced conventionally stored blood.

Embodiment 98. The method of Embodiment 97, wherein the albumin plus globulin (total protein) level is reduced to between 5 grams per deciliter (g/dL) and 10 g/dL after the administering.

Embodiment 99. The method of any one of Embodiments 89 to 98, wherein the patient has an interleukin 6 (IL-6) level that is reduced by at least 20% after the administering, compared to a TBI patient undergoing hemorrhagic shock having been administered the same amount of non-oxygen reduced conventionally stored blood.

Embodiment 100. The method of Embodiment 99, wherein the IL-6 level is reduced to between 3 picograms per milliliter (pg/mL) and 8 pg/mL of blood after the administering.

Embodiment 101. The method of any one of Embodiments 89 to 100, wherein the patient has a CXC motif chemokine ligand 1 (CXCL1) level that is reduced by at least 30% after the administering, compared to a TBI patient undergoing hemorrhagic shock having been administered the same amount of non-oxygen reduced conventionally stored blood.

Embodiment 102. The method of Embodiment 101, wherein the CXCL1 level is reduced to between 30 picograms per milliliter (pg/mL) and 90 pg/mL of serum after the administering.

Embodiment 103. The method of any one of Embodiments 89 to 102, wherein the patient has a monocyte chemoattractant protein-1 (MCP1) level of that is reduced by at least 20% after the administering, compared to a TBI patient undergoing hemorrhagic shock having been administered the same amount of non-oxygen reduced conventionally stored blood.

Embodiment 104. The method of Embodiment 103, wherein the MCP1 level is reduced to between 50 picograms per milliliter (pg/mL) and 200 pg/mL of plasma after the administering.

Embodiment 105. The method of any one of Embodiments 89 to 104, wherein the patient has a ferritin level that is reduced by between 5% to 20% after the administering, compared to a TBI patient undergoing hemorrhagic shock having been administered the same amount of non-oxygen reduced conventionally stored blood.

Embodiment 106. The method of Embodiment 105, wherein the ferritin level is reduced to between 5 milligrams per liter (mg/L) and 260 mg/L after the administering.

Embodiment 107. The method of any one of Embodiments 89 to 106, wherein the patient exhibits a Glasgow Coma Scale (GCS) score of 12 or less prior to the administering.

Embodiment 108. The method of Embodiment 107, wherein the Glasgow Coma Scale (GCS) score of the patient is between 3 and 12 prior to the administering.

Embodiment 109. The method of Embodiment 107 or Embodiment 108, wherein the Glasgow Coma Scale (GCS) score of the patient is between 13 and 15 after the administering.

Embodiment 110. The method of any one of Embodiments 89 to 109, wherein the patient has a shock index (SI) of greater than 0.9 prior to the administering.

Embodiment 111. The method of Embodiment 110, wherein the shock index (SI) of the patient is reduced to between 0.5 and 0.9 after the administering.

Embodiment 112. The method of any one of Embodiments 89 to 111, wherein the traumatic brain injury is stabilized and the hemorrhagic shock is reversed after the administering.

Embodiment 113. The method of any one of Embodiments 89 to 112, wherein the oxygen reduced blood has an oxygen saturation (SO₂) of 10% or less prior to and during storage.

Embodiment 114. The method of any one of Embodiments 89 to 113, wherein the administering is by transfusion.

Embodiment 115. A method of improving splenic function in a traumatic brain injury patient in need thereof, comprising administering oxygen reduced blood having an oxygen saturation (SO₂) of 20% or less prior to and during storage, wherein the patient has a traumatic brain injury accompanied by hemorrhagic shock, and wherein the patient has a monocyte chemoattractant protein-1 (MCP1) level that is reduced by at least 20% after the administering, compared to a TBI patient undergoing hemorrhagic shock having been administered the same amount of non-oxygen reduced conventionally stored blood.

Embodiment 116. The method of Embodiment 115, wherein the MCP1 level is reduced to between 50 picograms per milliliter (pg/mL) and 200 pg/mL after the administering.

Embodiment 117. The method of Embodiment 115 or Embodiment 116, wherein the patient has a ferritin level that is reduced by between 5% to 20% after the administering, compared to a TBI patient undergoing hemorrhagic shock having been administered the same amount of non-oxygen reduced conventionally stored blood.

Embodiment 118. The method of Embodiment 117, wherein the ferritin level is reduced to between 5 milligrams per liter (mg/L) and 260 mg/L after the administering.

Embodiment 119. The method of any one of Embodiments 115 to 118, wherein the patient exhibits a Glasgow Coma Scale (GCS) score of 12 or less prior to the administering.

Embodiment 120. The method of Embodiment 119, wherein the Glasgow Coma Scale (GCS) score of the patient is between 3 and 12 prior to the administering.

Embodiment 121. The method of Embodiment 119 or Embodiment 120, wherein the Glasgow Coma Scale (GCS) score of the patient is between 13 and 15 after the administering.

Embodiment 122. The method of any one of Embodiments 115 to 121, wherein the patient has a shock index (SI) of greater than 0.9 prior to the administering.

Embodiment 123. The method of Embodiment 122, wherein the shock index (SI) of the patient is reduced to between 0.5 and 0.9 after the administering.

Embodiment 124. The method of any one of Embodiments 115 to 123, wherein the traumatic brain injury is stabilized and the hemorrhagic shock is reversed after the administering.

Embodiment 125. The method of any one of Embodiments 115 to 124, wherein the oxygen reduced blood has an oxygen saturation (SO₂) of 10% or less prior to and during storage.

Embodiment 126. The method of any one of Embodiments 115 to 125, wherein the administering is by transfusion.

Embodiment 127. A method of improving cardiac function in a traumatic brain injury patient in need thereof, comprising administering oxygen reduced blood having an oxygen saturation (SO₂) of 20% or less prior to and during storage, wherein the patient has a traumatic brain injury accompanied by hemorrhagic shock, and wherein the patient has a troponin level that is reduced by of at least 5% after the administering, compared to a TBI patient undergoing hemorrhagic shock having been administered the same amount of non-oxygen reduced conventionally stored blood.

Embodiment 128. The method of Embodiment 127, wherein the troponin level is reduced to between 0.01 nanograms per milliliter (ng/mL) and 0.1 ng/mL after the administering.

Embodiment 129. The method of Embodiment 127 or Embodiment 128, wherein the patient has a plasma C reactive protein (CRP) level that is reduced by at least 5% after the administering, compared to a TBI patient undergoing hemorrhagic shock having been administered the same amount of non-oxygen reduced conventionally stored blood.

Embodiment 130. The method of Embodiment 129, wherein the plasma CRP level is reduced to between 5 milligrams per liter (mg/L) and 15 mg/L after the administering.

Embodiment 131. The method of any one of Embodiments 127 to 130, wherein the patient has a plasma atrial natriuretic peptide (ANP) level that is reduced by at least 5% after the administering, compared to a TBI patient undergoing hemorrhagic shock having been administered the same amount of non-oxygen reduced conventionally stored blood.

Embodiment 132. The method of Embodiment 131, wherein the plasma ANP level is reduced to between 5 picograms per milliliter (pg/mL) and 30 pg/mL after the administering.

Embodiment 133. The method of any one of Embodiments 127 to 132, wherein the patient has a ferritin level that is reduced by between 5% and 20% after the administering, compared to a TBI patient undergoing hemorrhagic shock having been administered the same amount of non-oxygen reduced conventionally stored blood.

Embodiment 134. The method of Embodiment 133, wherein the ferritin level is reduced to between 5 milligrams per liter (mg/L) and 260 mg/L after the administering.

Embodiment 135. The method of any one of Embodiments 127 to 134, wherein the patient has an interleukin-6 (IL-6) level that is reduced by at least 20% after the administering, compared to a TBI patient undergoing hemorrhagic shock having been administered the same amount of non-oxygen reduced conventionally stored blood.

Embodiment 136. The method of Embodiment 135, wherein the IL-6 level is reduced to between 3 picograms per milliliter (pg/mL) and 8 pg/mL of blood after administering.

Embodiment 137. The method of any one of Embodiments 127 to 136, wherein the patient has a tumor necrosis factor alpha (TNF-α) level that is reduced by at least 5% after the administering, compared to a TBI patient undergoing hemorrhagic shock having been administered the same amount of non-oxygen reduced conventionally stored blood.

Embodiment 138. The method of Embodiment 137, wherein the TNF-α level is reduced to between 50 picograms per milliliter (pg/mL) and 100 pg/mL after the administering.

Embodiment 139. The method of any one of Embodiments 127 to 138, wherein the patient has a monocyte chemoattractant protein-1 (MCP1) level that is reduced by at least 20% after the administering, compared to a TBI patient undergoing hemorrhagic shock having been administered the same amount of non-oxygen reduced conventionally stored blood.

Embodiment 140. The method of Embodiment 139, wherein the MCP1 level is reduced to between 50 picograms per milliliter (pg/mL) and 200 pg/mL of plasma after the administering.

Embodiment 141. The method of any one of Embodiments 127 to 140, wherein the patient has an cardiac output (CO) of that is increased by at least 2% after the administering.

Embodiment 142. The method of Embodiment 141, wherein the cardiac output (CO) is increased to between 1 liter per minute (L/min) and 10 L/min after the administering.

Embodiment 143. The method of any one of Embodiments 127 to 142, wherein the patient exhibits a Glasgow Coma Scale (GCS) score of 12 or less prior to the administering.

Embodiment 144. The method of Embodiment 143, wherein the Glasgow Coma Scale (GCS) score of the patient is between 3 and 12 prior to the administering.

Embodiment 145. The method of Embodiment 143 or Embodiment 144, wherein the Glasgow Coma Scale (GCS) score of the patient is between 13 and 15 after the administering.

Embodiment 146. The method of any one of Embodiments 127 to 145, wherein the patient has a shock index (SI) of greater than 0.9 prior to the administering.

Embodiment 147. The method of Embodiment 146, wherein the shock index (SI) of the patient is reduced to between 0.5 and 0.9 after the administering.

Embodiment 148. The method of any one of Embodiments 127 to 147, wherein the traumatic brain injury is stabilized and the hemorrhagic shock is reversed after the administering.

Embodiment 149. The method of any one of Embodiments 127 to 148, wherein the oxygen reduced blood has an oxygen saturation (SO₂) of 10% or less prior to and during storage.

Embodiment 150. The method of any one of Embodiments 127 to 149, wherein the administering is by transfusion.

Embodiment 151. A method of improving pulmonary function in a traumatic brain injury (TBI) patient in need thereof, comprising administering oxygen reduced blood having an oxygen saturation (SO₂) of 20% or less prior to and during storage, wherein the patient has a traumatic brain injury accompanied by hemorrhagic shock, and wherein the patient has a myeloperoxidase (MPO) level that is reduced after the administering, compared to a TBI patient undergoing hemorrhagic shock having been administered the same amount of non-oxygen reduced conventionally stored blood.

Embodiment 152. The method of Embodiment 151, wherein the MPO level is reduced to between 10 nanograms per milliliter (ng/mL) and 240 ng/mL after the administering.

Embodiment 153. The method of Embodiment 151 or Embodiment 152, wherein the patient has a lung neutrophil content that is reduced after the administering, compared to a TBI patient undergoing hemorrhagic shock having been administered the same amount of non-oxygen reduced conventionally stored blood.

Embodiment 154. The method of Embodiment 153, wherein the lung neutrophil content is reduced to between 40% and 70% after the administering.

Embodiment 155. The method of Embodiment 153 or Embodiment 154, wherein the lung neutrophil content is measured by flow cytometry.

Embodiment 156. The method of any one of Embodiments 151 to 155, wherein the patient exhibits a Glasgow Coma Scale (GCS) score of 12 or less prior to the administering.

Embodiment 157. The method of Embodiment 156, wherein the Glasgow Coma Scale (GCS) score of the patient is between 3 and 12 prior to the administering.

Embodiment 158. The method of Embodiment 156 or Embodiment 157, wherein the Glasgow Coma Scale (GCS) score of the patient is between 13 and 15 after the administering.

Embodiment 159. The method of any one of Embodiments 151 to 158, wherein the patient has a shock index (SI) of greater than 0.9 prior to the administering.

Embodiment 160. The method of Embodiment 159, wherein the shock index (SI) of the patient is reduced to between 0.5 and 0.9 after the administering.

Embodiment 161. The method of any one of Embodiments 151 to 160, wherein the traumatic brain injury is stabilized and the hemorrhagic shock is reversed after the administering.

Embodiment 162. The method of any one of Embodiments 151 to 161, wherein the oxygen reduced blood has an oxygen saturation (SO₂) of 10% or less prior to and during storage.

Embodiment 163. The method of any one of Embodiments 151 to 162, wherein the administering is by transfusion.

Embodiment 164. A method of improving renal function in a traumatic brain injury (TBI) patient in need thereof, comprising administering oxygen reduced blood having an oxygen saturation (SO₂) of 20% or less prior to and during storage, wherein the patient has a traumatic brain injury accompanied by hemorrhagic shock, and wherein the patient has a urine creatinine level that is reduced by at least 5% after the administering, compared to a TBI patient undergoing hemorrhagic shock having been administered the same amount of non-oxygen reduced conventionally stored blood.

Embodiment 165. The method of Embodiment 164, wherein the urine creatinine level is reduced to between 10 milligrams per deciliter (mg/dL) and 350 mg/dL after the administering.

Embodiment 166. The method of Embodiment 164 or Embodiment 165, wherein the patient exhibits a Glasgow Coma Scale (GCS) score of 12 or less prior to the administering.

Embodiment 167. The method of Embodiment 166, wherein the Glasgow Coma Scale (GCS) score of the patient is between 3 and 12 prior to the administering.

Embodiment 168. The method of Embodiment 166 or Embodiment 167, wherein the Glasgow Coma Scale (GCS) score of the patient is between 13 and 15 after the administering.

Embodiment 169. The method of any one of Embodiments 164 to 168, wherein the patient has a shock index (SI) of greater than 0.9 prior to the administering.

Embodiment 170. The method of Embodiment 169, wherein the shock index (SI) of the patient is reduced to between 0.5 and 0.9 after the administering.

Embodiment 171. The method of any one of Embodiments 164 to 170, wherein the traumatic brain injury is stabilized and the hemorrhagic shock is reversed after the administering.

Embodiment 172. The method of any one of Embodiments 164 to 171, wherein the oxygen reduced blood has an oxygen saturation (SO₂) of 10% or less prior to and during storage.

Embodiment 173. The method of any one of Embodiments 164 to 172, wherein the administering is by transfusion.

Embodiment 174. A method of increasing a plasma serum cytokine level in a traumatic brain injury (TBI) patient in need thereof, comprising administering oxygen reduced blood having an oxygen saturation (SO₂) of 20% or less prior to and during storage, wherein the patient has a traumatic brain injury accompanied by hemorrhagic shock, and wherein the plasma serum cytokine is selected from the group consisting of interleukin 6 (IL-6), CXC motif chemokine ligand 1 (CXCL1), interleukin 10 (IL-10), and any combination thereof.

Embodiment 175. The method of Embodiment 174, wherein the plasma serum cytokine is IL-10, and wherein the plasma level of IL-10 in the patient is increased by at least 5% after the administering, compared to a TBI patient undergoing hemorrhagic shock having been administered the same amount of non-oxygen reduced conventionally stored blood.

Embodiment 176. The method of Embodiment 175, wherein the plasma level of IL-10 is increased to between 3 picograms per milliliter (pg/mL) and 12 pg/mL after the administering.

Embodiment 177. The method of any one of Embodiments 174 to 176, wherein the patient exhibits a Glasgow Coma Scale (GCS) score of 12 or less prior to the administering.

Embodiment 178. The method of Embodiment 177, wherein the Glasgow Coma Scale (GCS) score of the patient is between 3 and 12 prior to the administering.

Embodiment 179. The method of Embodiment 177 or Embodiment 178, wherein the Glasgow Coma Scale (GCS) score of the patient is between 13 and 15 after the administering.

Embodiment 180. The method of any one of Embodiments 174 to 179, wherein the patient has a shock index (SI) of greater than 0.9 prior to the administering.

Embodiment 181. The method of Embodiment 180, wherein the shock index (SI) of the patient is reduced to between 0.5 and 0.9 after the administering.

Embodiment 182. The method of any one of Embodiments 174 to 181, wherein the traumatic brain injury is stabilized and the hemorrhagic shock is reversed after the administering.

Embodiment 183. The method of any one of Embodiments 174 to 182, wherein the oxygen reduced blood has an oxygen saturation (SO₂) of 10% or less prior to and during storage.

Embodiment 184. The method of any one of Embodiments 174 to 183, wherein the administering is by transfusion.

Embodiment 185. A method of modulating a stress level marker in the plasma of a traumatic brain injury (TBI) patient in need thereof, comprising administering oxygen reduced blood having an oxygen saturation (SO₂) of 20% or less prior to and during storage, wherein the patient has a traumatic brain injury accompanied by hemorrhagic shock, and wherein the stress level marker is selected from the group consisting of epinephrine, norepinephrine, cortisol, and any combination thereof.

Embodiment 186. The method of Embodiment 185, wherein the stress level marker is epinephrine, and wherein the plasma level of epinephrine in the patient is reduced by at least 5% after the administering, compared to a TBI patient undergoing hemorrhagic shock having been administered the same amount of non-oxygen reduced conventionally stored blood.

Embodiment 187. The method of Embodiment 186, wherein the plasma level of epinephrine is reduced to between 5 picograms per milliliter (pg/mL) and 150 pg/mL after the administering.

Embodiment 188. The method of Embodiment 185, wherein the stress level marker is norepinephrine, and wherein the plasma level of norepinephrine in the patient is reduced by at least 5% after the administering, compared to a TBI patient undergoing hemorrhagic shock having been administered the same amount of non-oxygen reduced conventionally stored blood.

Embodiment 189. The method of Embodiment 188, wherein the plasma level of norepinephrine is reduced to between 50 picograms per milliliter (pg/mL) and 2000 pg/mL after the administering.

Embodiment 190. The method of Embodiment 185, wherein the stress level marker is cortisol, and wherein the plasma level of cortisol in the patient is reduced by at least 5% after the administering, compared to a TBI patient undergoing hemorrhagic shock having been administered the same amount of non-oxygen reduced conventionally stored blood.

Embodiment 191. The method of Embodiment 190, wherein the plasma level of cortisol is increased to between 2 milligrams per deciliter (mg/dL) and 25 mg/dL after the administering.

Embodiment 192. The method of any one of Embodiments 185 to 191, wherein the patient exhibits a Glasgow Coma Scale (GCS) score of 12 or less prior to the administering.

Embodiment 193. The method of Embodiment 192, wherein the Glasgow Coma Scale (GCS) score of the patient is between 3 and 12 prior to the administering.

Embodiment 194. The method of Embodiment 192 or Embodiment 193, wherein the Glasgow Coma Scale (GCS) score of the patient is between 13 and 15 after the administering.

Embodiment 195. The method of any one of Embodiments 185 to 194, wherein the patient has a shock index (SI) of greater than 0.9 prior to the administering.

Embodiment 196. The method of Embodiment 195, wherein the shock index (SI) of the patient is reduced to between 0.5 and 0.9 after the administering.

Embodiment 197. The method of any one of Embodiments 185 to 196, wherein the traumatic brain injury is stabilized and the hemorrhagic shock is reversed after the administering.

Embodiment 198. The method of any one of Embodiments 185 to 197, wherein the oxygen reduced blood has an oxygen saturation (SO₂) of 10% or less prior to and during storage.

Embodiment 199. The method of any one of Embodiments 185 to 198, wherein the administering is by transfusion.

Embodiment 200. A method of increasing tissue oxygenation in one or more vital organs of a traumatic brain injury (TBI) patient in need thereof, comprising administering oxygen reduced blood having an oxygen saturation (SO₂) of 20% or less prior to and during storage, wherein the patient has a traumatic brain injury accompanied by hemorrhagic shock, and wherein the one or more vital organs are selected from the group consisting of the kidney, the liver, the lungs, the spleen, the heart, and any combination thereof.

Embodiment 201. The method of Embodiment 200, wherein the tissue oxygen saturation in each of the one or more vital organs is increased by at least 10%.

Embodiment 202. The method of Embodiment 201, wherein the tissue oxygen saturation in each of the one or more vital organs is increased by at least 25%.

Embodiment 203. The method of any one of Embodiments 200 to 202, wherein the tissue oxygen saturation is measured by administering an effective amount of pimonidazole to the patient and detecting the distribution of the pimonidazole hydrochloride in the one or more vital organs of the patient.

Embodiment 204. The method of any one of Embodiments 200 to 203, wherein the patient exhibits a Glasgow Coma Scale (GCS) score of 12 or less prior to the administering.

Embodiment 205. The method of Embodiment 204, wherein the Glasgow Coma Scale (GCS) score of the patient is between 3 and 12 prior to the administering.

Embodiment 206. The method of Embodiment 204 or embodiment 205, wherein the Glasgow Coma Scale (GCS) score of the patient is between 13 and 15 after the administering.

Embodiment 207. The method of any one of Embodiments 200 to 206, wherein the patient has a shock index (SI) of greater than 0.9 prior to the administering.

Embodiment 208. The method of Embodiment 207, wherein the shock index (SI) of the patient is reduced to between 0.5 and 0.9 after the administering.

Embodiment 209. The method of any one of Embodiments 200 to 208, wherein the traumatic brain injury is stabilized and the hemorrhagic shock is reversed after the administering.

Embodiment 210. The method of any one of Embodiments 200 to 209, wherein the oxygen reduced blood has an oxygen saturation (SO₂) of 10% or less prior to and during storage.

Embodiment 211. The method of any one of Embodiments 200 to 210, wherein the administering is by transfusion.

### EXAMPLES

### Example 1: Collection of Blood and Sample Preparation

Red blood cells are collected from male Sprague Dawley donor rats (Charles River Laboratories, Wilmington, MA) weighing 400-450 grams (g). Briefly, animals are anesthetized by administering 5%/vol isoflurane in compressed room air (Drägerwerk AG, Lübeck, Germany) to induce anesthesia, then maintained at 2.5%/vol isoflurane thereafter. Animals are placed on a heating pad to maintain core body temperature at 37 °C and allowed to breathe freely from a nosecone delivering the anesthetic. Blood is drained using a heparinized 18 gauge (G) needle and syringe through a cardiac puncture created in the anesthetized animal. After blood is drained, the animal is euthanized by intravenously administering Euthasol (a solution of pentobarbital sodium and phenytoin sodium) at 300 milligrams (mg) per kilogram (kg).

Collected blood is then pooled into 50 millimeters (mL) conical tubes containing 0.14 mL of citrate phosphate double dextrose (CP2D) (Haemonetics Corp, Union, SC) for every 1 mL of whole blood. The tubes containing blood are soft-spun at about 1000 g force (equal to 2600 revolutions per minute (RPM)) on an IEC Centra CL2 Centrifuge to remove the topmost layer containing platelet-rich plasma (PRP). The blood is then pooled in a 150 mL Fenwal 4R2001 transfer bag (Fresenius Kabi, Bad Homburg, Germany) containing 0.22 mL of additive solution-3 (AS-3) (Haemonetics Corp, Union, SC) for every 1 mL of blood. The Fenwal transfer bag is connected to a NEO High-Efficiency Leukocyte Reduction Filter (Haemonetics Corp, Union, SC) to remove leukocytes.

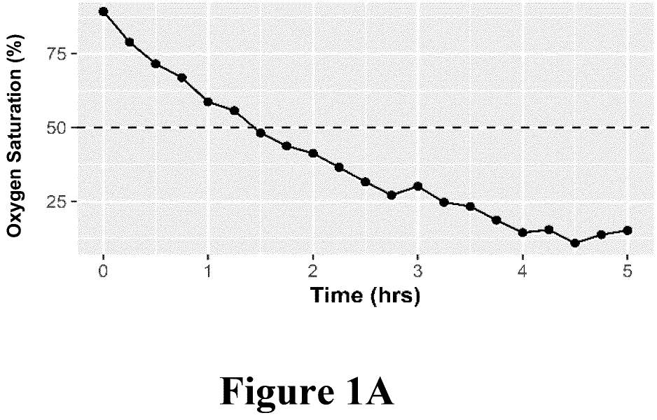

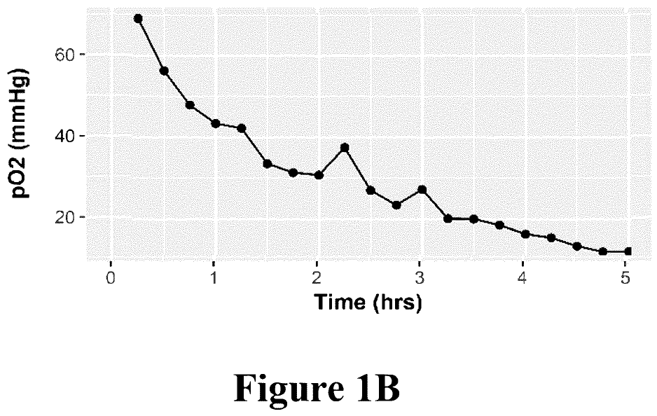

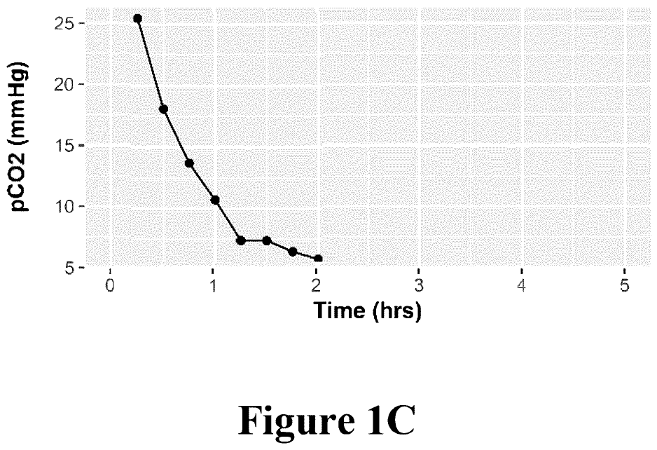

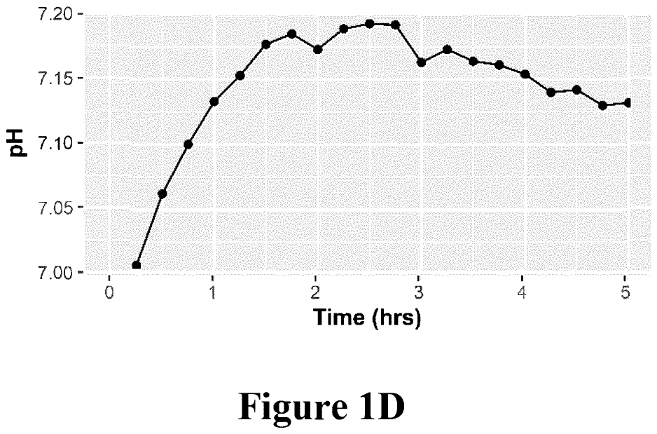

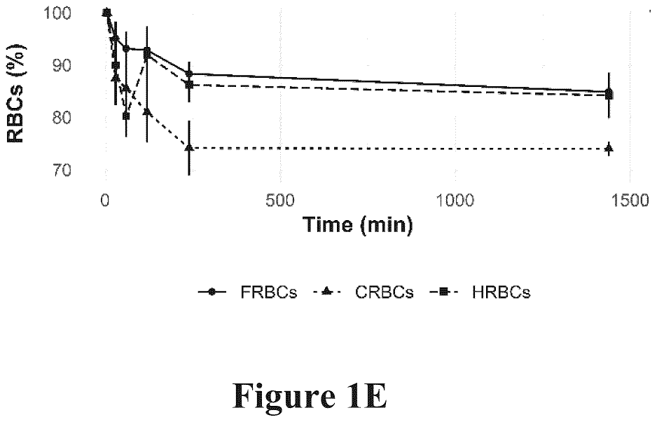

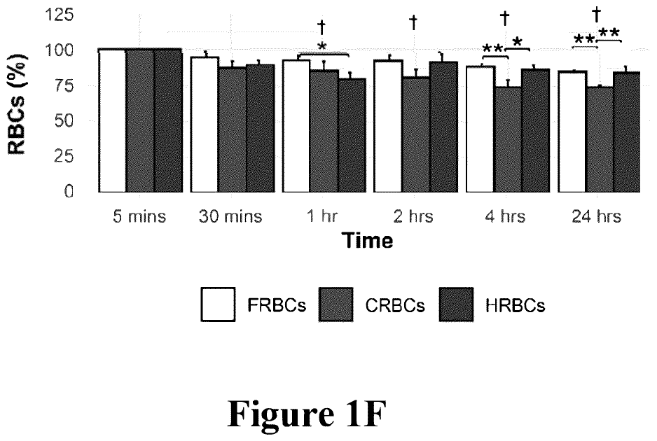

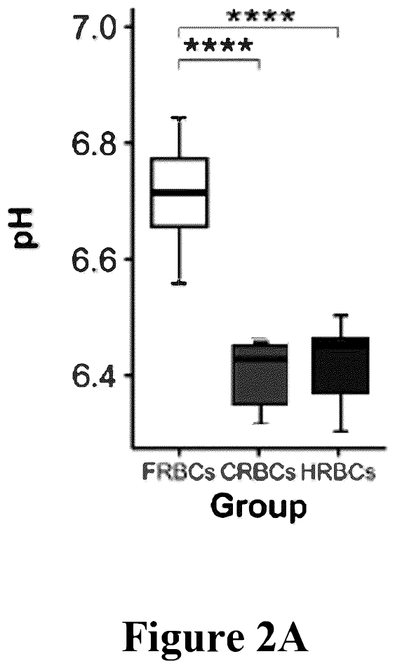

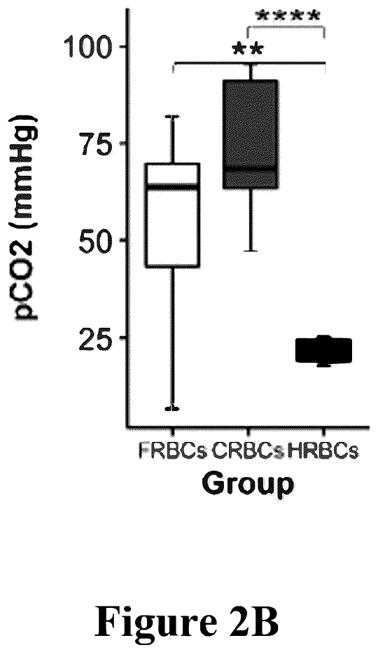

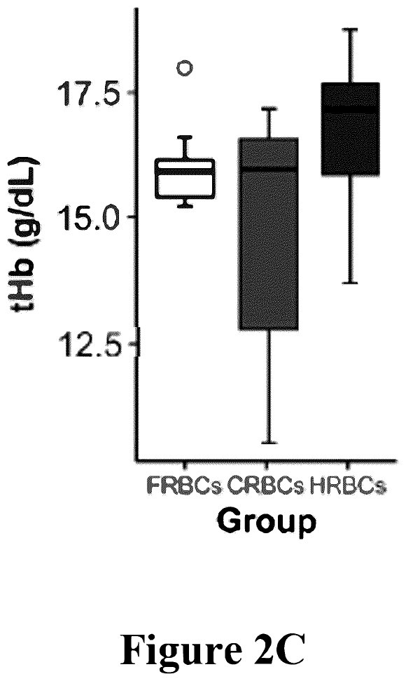

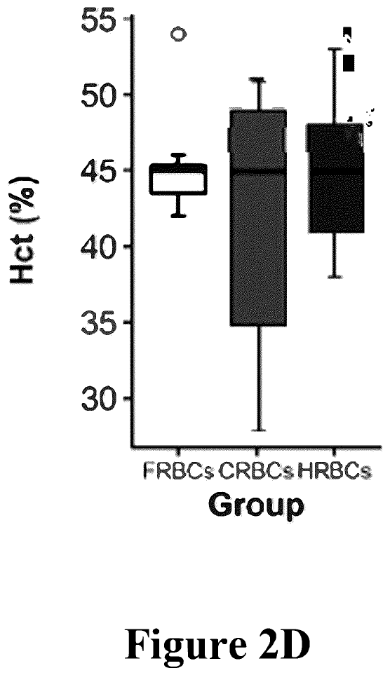

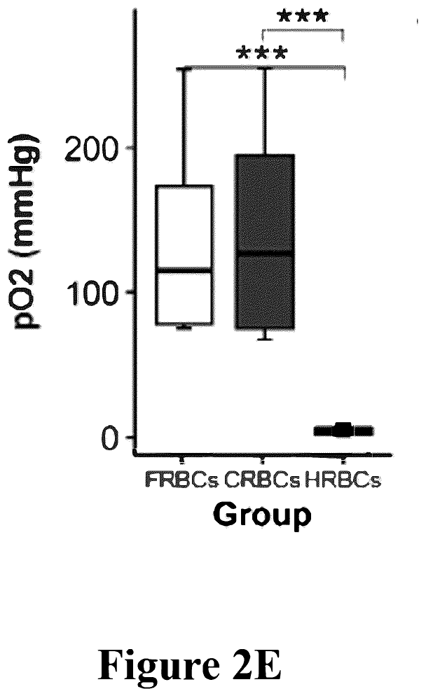

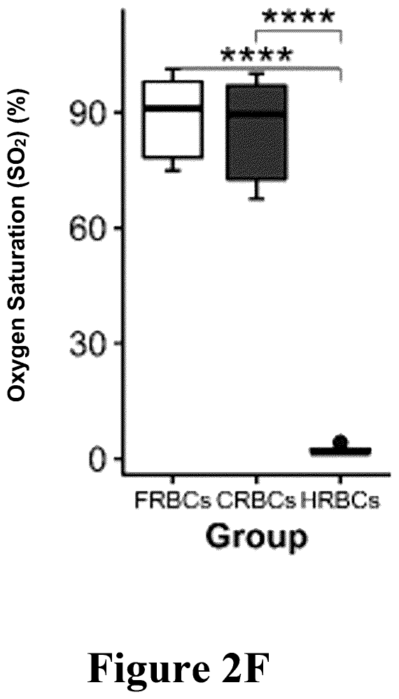

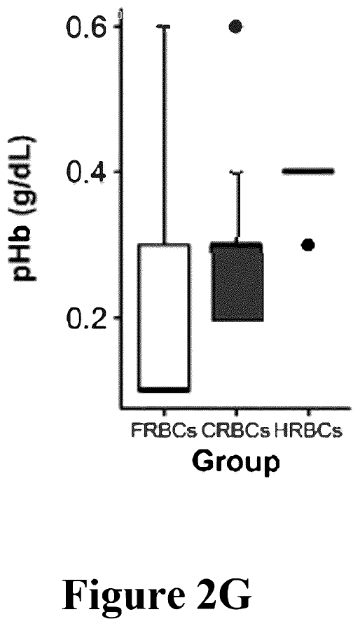

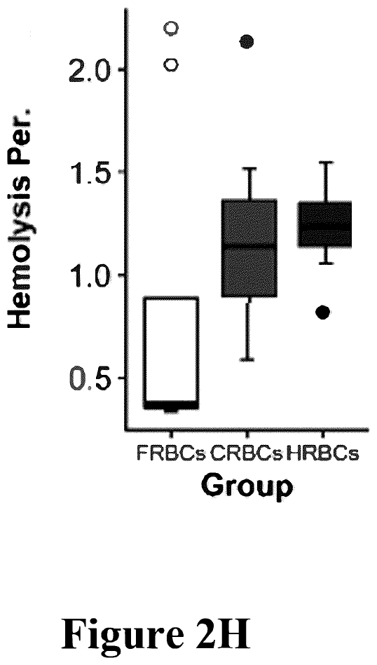

The leukoreduced pooled blood is split into three groups before further processing steps: fresh red blood cells (FRBCs), conventionally stored red blood cells (CRBCs), and hypoxically stored red blood cells (HRBCs). CRBCs are stored as leukoreduced pooled blood (i.e., without further processing steps to remove oxygen and reduce levels of hemoglobin oxygen saturation and partial pressure of oxygen) in a segmented Fenwal transfer bag until the start of the study (see Example 3). HRBCs are subjected to further processing steps to remove oxygen and reduce levels of hemoglobin oxygen saturation (SO₂) and partial pressure of oxygen (pO₂; measured in millimeters of mercury (mmHg)), thereby producing oxygen reduced blood. To remove oxygen and reduce levels of hemoglobin SO₂ and pO₂, HRBCs are transferred to an oxygen depletion device (see International Publication No. WO 2016/145210, hereby incorporated by reference in its entirety) and placed on a linear table shaker driven by a NSH-34RH motor (Bodine Electric Company, Northfield, IL) controlled by a MotorMaster 20000 series adjustable speed drive (Minarik Electric Company, Glendale, CA) set to 60 RPM for 5 hours. Samples of the blood are taken every 15 minutes from the oxygen depletion device to measure changes in blood gasses over the course of deoxygenation. As shown in FIGS. 1A-1B, oxygen saturation (SO₂) of blood decreases from about 90% to about 15% over 5 hours. Partial pressure of oxygen (pO₂) also decreases during the same time period, from about 70 mmHg to about 12 mmHg. As shown in FIG. 1C, partial pressure of carbon dioxide (pCO₂) decreases from about 26 mmHg to less than about 5 mmHg within two hours, at which point the levels of carbon dioxide are outside the range detectable by the ABL90 blood gas analyzer machine. As shown in FIG. 1D, pH increases from about 7 to about 7.13 over five hours due to reduction in carbon dioxide. The deoxygenated HRBCs are transferred to segmented Fenwal bags, which are in turn placed inside Mylar bags along with KIND oxygen sorbent packets to scavenge residual oxygen and carbon dioxide from the bags. CRBCs and HRBCs are stored for 3 weeks at 4 °C. FRBCs are processed in the same manner as described above for CRBCs, but are not stored for 3 weeks. Prior to the start of the study (see Example 3), a small amount (80 microliters (μL)) of blood is sampled from the Fenwal bags to determine properties of blood at time of transfusion using an ABL90 FLEX blood analyzer machine (Radiometer, Copenhagen, Denmark). As shown in FIGS. 2B, 2E, and 2F, pCO₂, pO₂, and SO₂ are all significantly lower in HRBCs compared to FRBCs and CRBCs. FIG. 2A shows that pH of FRBCs is significantly higher compared to CRBCs and HRBCs. There are no significant differences in total hemoglobin (tHb), hematocrit (Hct), or plasma hemoglobin (pHb) between groups. See FIGS. 2C, 2D, and 2G. As shown in FIGS. 2E-2F, at the time of transfusion, SO₂ and pO₂ of HRBCs are close to zero.

### Example 2: Recovery of Red Blood Cells (RBCs)

A small volume (around 200 μL) of FRBCs, CRBCs, and HRBCs stored for 24 hours is labeled with technetium-99. Labeled blood is intravenously administered to Sprague-Dawley rats (n=2 per group) and circulating radioactivity is measured via blood samples taken from the animals at several timepoints, including 5 minutes and 24 hours post-administration in order to estimate the fraction of administered RBCs surviving 24 hours after administration. See FIGS. 1E and 1F. Blood samples are taken via tail clip at 5 minutes and 24 hours post-administration. Blood samples are counted simultaneously using a Cobra II gamma counter (Packard Instrument Co., Meriden, CT).

### Example 3: Rat Model of Resuscitation after Traumatic Brain Injury Accompanied by Hemorrhagic Shock

Studies are performed in male Wistar rats (Charles River Laboratories, Wilmington, MA) weighing 350-400 g. Animal handling and care follows the National Institutes of Health (NIH) Guide for Care and Use of Laboratory Animals, and all protocols are approved by the University of California San Diego Institutional Animal Care and Use Committee. All methods are carried out in accordance with the Animal Research: Reporting of In Vivo Experiments (ARRIVE) guidelines. Animals are split into two groups: Control (C) and traumatic brain injury accompanied by hemorrhagic shock (TBI+HS). C animals serve as control for measuring organ damage markers in harvested tissues. Blood and hemodynamics measurements are taken from TBI+HS animals at baseline (also referred to as “Bl” herein), prior to induction of TBI+HS. Briefly, animals are anesthetized by administering 5%/vol isoflurane in compressed room air (Drägerwerk AG, Lübeck, Germany) to induce anesthesia, then maintained at 1.5%/vol isoflurane thereafter, except for an increase to 2.5%/vol isoflurane during surgical procedure. Animals are placed on a heating pad to preserve core body temperature at 37 °C and allowed to freely breathe from a nosecone delivering the anesthetic. Animals are instrumented with a right femoral artery catheter for hemodynamic assessment and a right femoral vein catheter for blood withdrawal and intravenous infusion. Animals are allowed to stabilize for 10 minutes. Baseline measurements are collected upon stabilization.

Before induction of traumatic brain injury (TBI), animals are transferred to a stereotaxic apparatus and placed in the ventral position. Isoflurane is increased to 2.5%/vol for 5 minutes before induction of TBI. To induce TBI, a 5 millimeter (mm) craniotomy is performed over the animal's right cerebral cortex, and the dura is impacted with a 5.0 mm flat-tipped impactor at a velocity of 5 meters (m) per second and a dwell time of 200 microseconds (ms) via a pneumatically controlled cortical impactor (CCI) (Leica Biosystems, Vista, CA). After impaction, the head opening is surgically closed and the animals are placed back on the heating pad in the dorsal position. Isoflurane is decreased to 1.5%/vol for 10 minutes before induction of hemorrhagic shock (HS).

Before induction of HS, animals are given 10 minutes to stabilize after cortical impaction. Animals are intravenously heparinized by administering 100 international units (IU) per kg of heparin to ensure patency of the catheters during the study. Animals are hemorrhaged by withdrawing blood from the femoral vein catheter until mean arterial pressure (MAP) reaches 40 mmHg, thereby placing the animals in a severe hypovolemic shock condition. The hypovolemic shock condition is maintained at a MAP of between 35 and 40 mmHg for 90 minutes by alternately withdrawing or returning small volumes of blood when the MAP is out of the indicated range for more than 2 minutes. Animals are then randomly assigned to either the FRBC, CRBC, or HRBC group (n=9 animals per group). Resuscitation (also referred to as reperfusion herein) is implemented by infusion of previously processed and stored RBCs (FRBCs, CRBCs, or HRBCs; see Example 1) at a rate of 2 mL per minute until the amount of infused blood reaches about 70% of the amount of blood withdrawn during the HS procedure (generally about 7 mL).

Animals are monitored for 120 minutes from the beginning of resuscitation until euthanasia at the end of the study. Blood samples are taken from the animals at baseline (Bl), 90 minutes into HS (also referred to as “Shock” herein), 30 minutes after resuscitation, and 2 hours after resuscitation. Arterial blood is collected in heparinized capillary tubes and centrifuged to measure hematocrit. Arterial and venous blood is collected in heparinized capillary tubes and immediately analyzed for partial pressure of oxygen (pO₂), partial pressure of carbon dioxide (pCO₂), pH, oxygen saturation (SO₂), glucose, and lactate (ABL90; Radiometer America, Brea, CA). Arterial pressure and heart rate (HR) is recorded continuously from the femoral artery catheter (MP150, Biopac, Santa Barbara, CA) at a 2 kilohertz (kHz) sampling rate. Blood pressure recordings are used to calculate MAP, systolic blood pressure (SBP), and diastolic blood pressure (DBP) using commercial software (AcqKnowledge; Biopac, Santa Barbara, CA).

Two hours after resuscitation, 10 mL of blood is collected from the femoral artery catheter and centrifuged to separate plasma. At the end of the study, animals are euthanized with Euthasol and the following samples and organs are harvested: (i) urine; (ii) kidneys; (iii) liver; (iv) spleen; (v) heart; and (vi) lungs. The samples and organs are evaluated for markers of inflammation, organ function, organ injury, and oxidative stress using enzyme-linked immunoassay (ELISA) methods and flow cytometry performed on tissue homogenates and plasma. See Table 1.

TABLE 1
ELISA kits used for analysis.

| | | | Source of | |
|---|---|---|---|---|
| Method | Kit/Assay | Analyte | sample | Vendor |
| ELISA | KA1625 | AST | Plasma | Abnova Corp, |
| | | | | Taiwan |
| ELISA | KA4189 | ALT | Plasma | Abnova Corp, |
| | | | | Taiwan |
| ELISA | BMS625 | IL-6 | Plasma and heart | Thermo Fisher, |
| | | | | Waltham, MA |
| ELISA | BMS629 | IL-10 | Plasma | Thermo Fisher, |
| | | | | Waltham, MA |
| ELISA | KB02-H2 | Creatinine | Plasma and urine | Arbor Assays |
| | | | | Inc, Ann Arbor, |
| | | | | MI |
| ELISA | K024-H5 | BUN | Plasma | Arbor Assays |
| | | | | Inc, Ann Arbor, |
| | | | | MI |
| ELISA | ERCXCL1 | CXCL1 | Plasma, lung, | Thermo Fisher, |
| | | | liver | Waltham, MA |
| ELISA | MCA-155 | Ferritin | Plasma, liver, | Serotec, Oxford, |
| | | | spleen and heart | UK |
| ELISA | BA-E-6600 | Catecholamines | Plasma | ImmunoSmol, |
| | | | | France |
| ELISA | ERLCN2 | Urine NGAL | Urine | Thermo Fisher, |
| | | | | Waltham, MA |
| ELISA | ab235627 | Bilirubin | Plasma | Abcam, |
| | | | | Cambridge, UK |
| Luminex | RECYTNMAG- | MCP-1 | Heart | Millipore |
| technology/ | 65K | | | Corporation, |
| Milliplex | | | | Massachusetts, |
| Immunoassay | | | | USA |
| Luminex | RECYTNMAG- | TNF-α | Heart | Millipore |
| technology/ | 65K | | | Corporation, |
| Milliplex | | | | Massachusetts, |
| Immunoassay | | | | USA |
| ELISA | ab246529 | Cardiac | Heart | Abcam, |
| | | Troponin | | Cambridge, UK |
| ELISA | Ab256398 | CRP | Plasma | Abcam, |
| | | | | Cambridge, UK |
| ELISA | Ab108797 | ANP | Plasma | Abcam, |
| | | | | Cambridge, UK |

### Example 4: Hemodynamics Analysis in a Rat Model of Resuscitation after Traumatic Brain Injury Accompanied by Hemorrhagic Shock

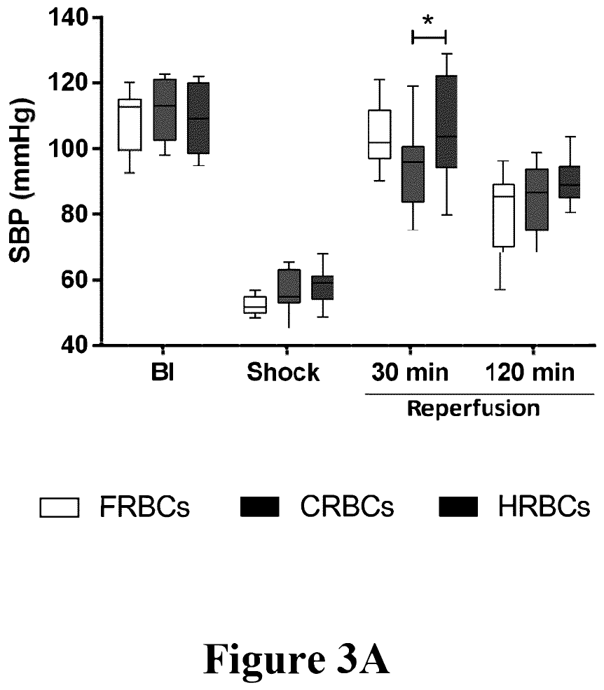

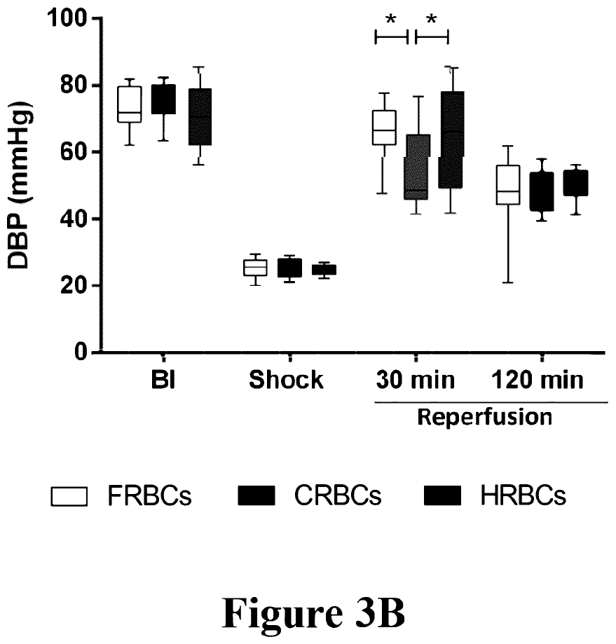

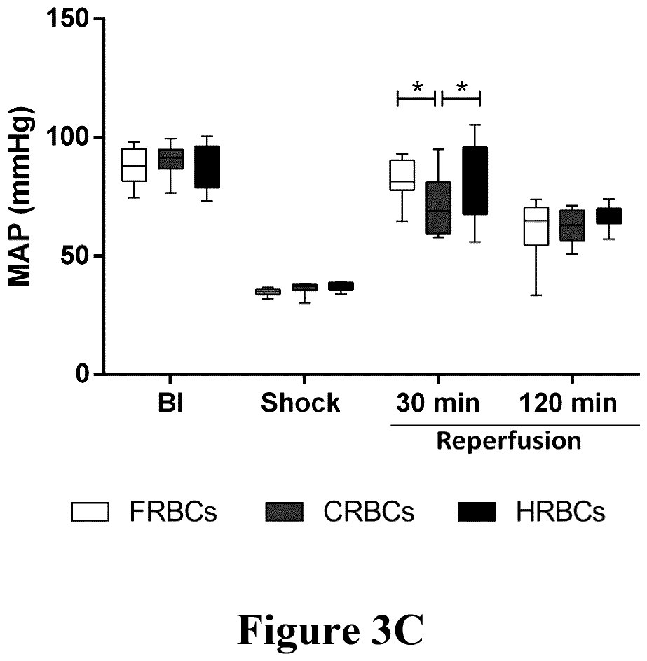

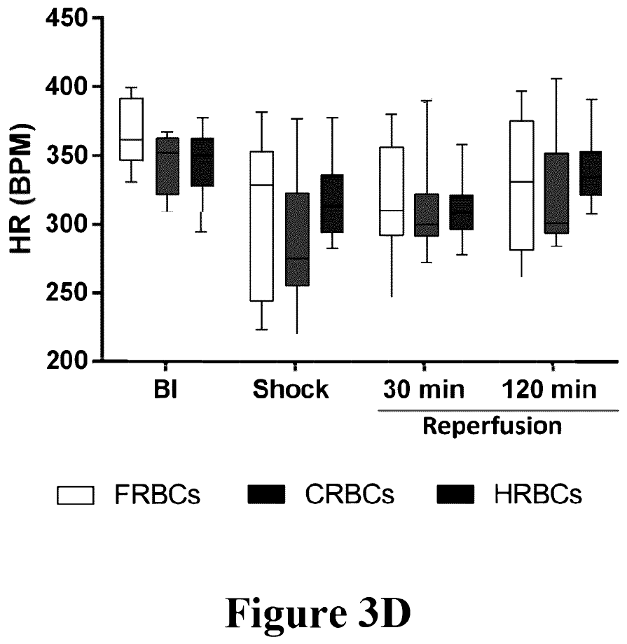

Systolic blood pressure (SBP), diastolic blood pressure (DBP), and mean arterial pressure (MAP) are reduced after hemorrhagic shock is induced. Providing FRBCs, CRBCs, or HRBCs increases SBP, DBP, and MAP values at 30 minutes after resuscitation. At 30 minutes after resuscitation, DBP and MAP are lower for CRBCs compared to FRBCs and HRBCs. See FIGS. 3A-3D. As shown in FIGS. 3A-3C, FRBCs and HRBCs provide a larger increase in SBP, DBP, and MAP values compared to CRBCs at 30 minutes after resuscitation. Similarly to FRBCs, HRBCs restore DBP and MAP values to a near baseline level at 30 minutes after resuscitation. See FIGS. 3B and 3C. Importantly, DBP and MAP are similar for FRBCs and HRBCs 30 and 120 mins after reperfusion. Moreover, HRBCs provide higher SBP compared to CRBCs. See FIG. 3A. No differences are observed in heart rate (HR) for FRBCs, CRBCs, or HRBCs. See FIG. 3D. At 120 minutes after resuscitation, all evaluated hemodynamics parameters reach the same level regardless of type of blood used.

FRBCs and HRBCs provide increased pO₂ levels compared to CRBCs at 120 minutes after resuscitation. See Table 2. There are no differences in pH, pCO₂, O₂ saturation, hematocrit, and total hemoglobin between the groups at 30 minutes into resuscitation, and there are no changes in any measurements at two hours after resuscitation. Lactate increases in all groups during hemorrhagic shock, and decreases similarly after resuscitation with FRBCs, CRBCs, or HRBCs.

TABLE 2
Hematological parameters.

| | FRBCs | CRBCs | HRBCs |
|---|---|---|---|
| tHb (g/dL) | | | |
| Baseline (Bl) | 13.7 ± 0.3 | 14.9 ± 0.3 | 14.4 ± 0.4 |
| Shock | 10.2 ± 0.2 | 10.7 ± 0.7 | 9.8 ± 0.2 |
| 30 min | 14.9 ± 0.4 | 15.4 ± 0.7 | 15.9 ± 0.5 |
| 120 min | 14.5 ± 0.3 | 14.8 ± 0.8 | 15.6 ± 0.5 |
| pH | | | |
| Baseline (Bl) | 7.371 ± 0.007 | 7.398 ± 0.010 | 7.373 ± 0.013 |
| Shock | 7.222 ± 0.057 | 7.301 ± 0.021 | 7.295 ± 0.013 |
| 30 min | 7.237 ± 0.055 | 7.313 ± 0.042 | 7.306 ± 0.016 |
| 120 min | 7.354 ± 0.018 | 7.346 ± 0.038 | 7.388 ± 0.013 |
| pCO₂ (mmHg) | | | |
| Baseline (Bl) | 44.5 ± 1.7 | 42.4 ± 1.6 | 45.8 ± 1.6 |
| Shock | 31.1 ± 2.3 | 30.7 ± 2.2 | 29.4 ± 1.9 |
| 30 min | 40.1 ± 2.2 | 38.2 ± 1.8 | 39.1 ± 1.1 |
| 120 min | 38.8 ± 1.0 | 34.3 ± 2.1 | 37.7 ± 1.2 |
| pO₂ (mmHg) | | | |
| Baseline (Bl) | 87.4 ± 3.8 | 84.0 ± 5.3 | 90.6 ± 3.0 |
| Shock | 116.1 ± 4.7 | 107.2 ± 2.4 | 118.3 ± 2.1 |
| 30 min | 93.2 ± 5.2 | 78.2 ± 7.2* | 94.4 ± 2.2† |
| 120 min | 92.0 ± 2.8 | 88.2 ± 5.1 | 96.6 ± 2.0 |
| O₂ sat. (%) | | | |
| Baseline (Bl) | 92.2 ± 0.8 | 91.1 ± 3.7 | 93.2 ± 1.9 |
| Shock | 94.2 ± 1.3 | 95.2 ± 0.5 | 99.0 ± 2.1 |
| 30 min | 90.8 ± 1.5 | 88.4 ± 4.7 | 96.7 ± 1.5 |
| 120 min | 93.6 ± 0.6 | 94.5 ± 0.8 | 89.1 ± 6.3 |
| Lactate (mmol/L) | | | |
| Baseline (Bl) | 1.1 ± 0.1 | 0.9 ± 0.1 | 1.0 ± 0.2 |
| Shock | 8.2 ± 1.7 | 6.9 ± 1.2 | 7.5 ± 0.8 |
| 30 min | 5.5 ± 1.5 | 3.9 ± 1.2 | 4.2 ± 0.9 |
| 120 min | 1.8 ± 0.3 | 2.2 ± 0.5 | 1.9 ± 0.3 |
| Hematocrit (%) | | | |
| Baseline (Bl) | 41.4 ± 1.1 | 46.3 ± 0.8 | 44.0 ± 1.5 |
| Shock | 32.4 ± 0.6 | 31.3 ± 1.2 | 30.9 ± 0.9 |
| 30 min | 45.2 ± 1.1 | 43.8 ± 1.6 | 44.6 ± 1.0 |
| 120 min | 43.8 ± 0.8 | 44.1 ± 2.1 | 44.0 ± 1.1 |

Data are presented as mean ± standard error (SE).
*= p < 0.05 compared to FRBCs.
†= p < 0.05 compared to CRBCs.
Fresh red blood cells (FRBCs), n = 9; conventionally stored red blood cells (CRBCs), n = 8; hypoxically stored red blood cells (HRBCs), n = 9.

### Example 5: Vital Organ Damage, Inflammation, and Function

Animals are analyzed to assess organ damage and function after TBI+HS and resuscitation. Although blood transfusion is the standard treatment after hemorrhagic shock, transfusion of stored blood is associated with adverse events, microhemodynamic aberration, and cardiovascular risk, resulting in decreased survival.

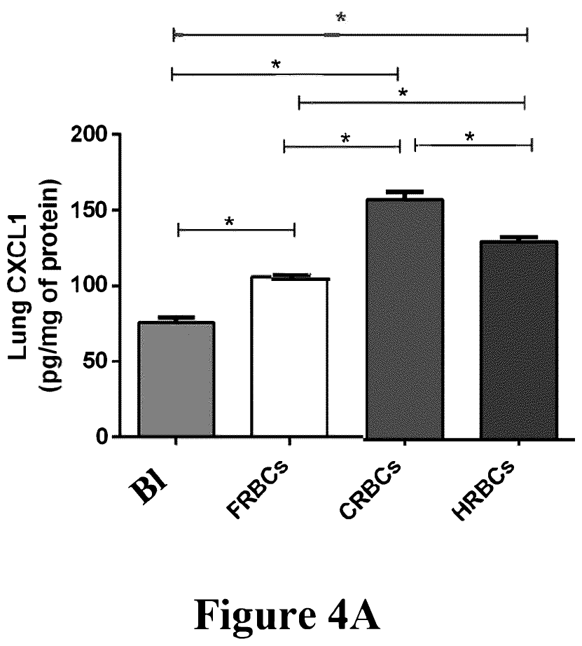

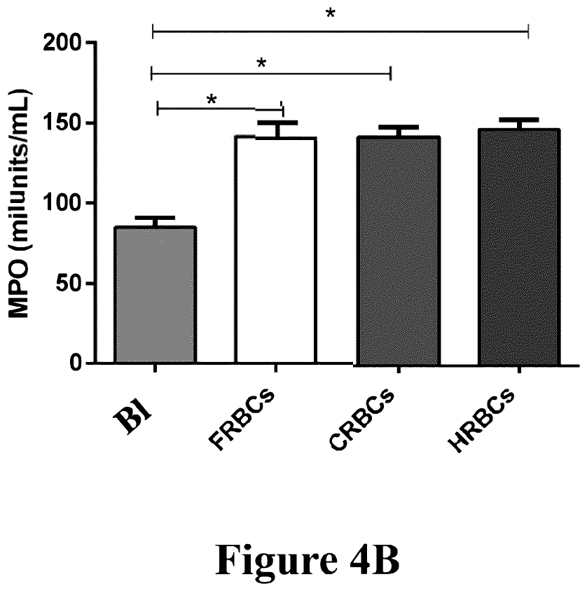

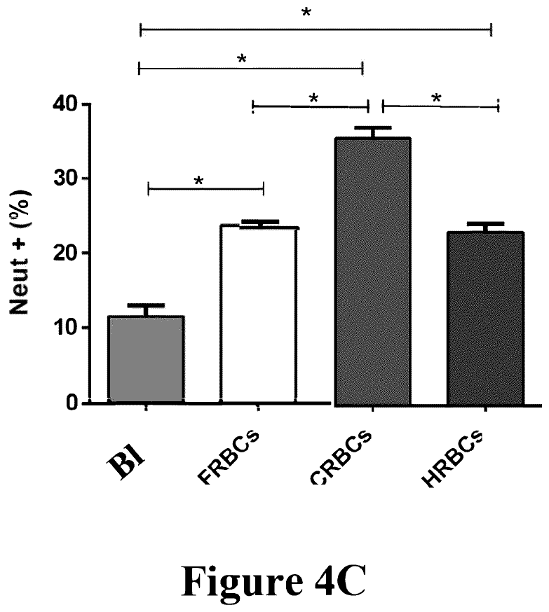

Elevated levels of inflammation markers in the lung can signify some form of lung damage or injury. Levels of lung CXC motif chemokine ligand 1 (CXCL1), myeloperoxidase, and CD45+ neutrophils (also referred to as “neut+” herein) are analyzed to determine lung damage. Resuscitation after TBI+HS increases levels of CXCL1, MPO, and CD45+ neutrophils in the lung compared to Bl. See FIGS. 4A-4C. Resuscitation with FRBCs and HRBCs reduces levels of CXCL1 and CD45+ neutrophils in the lung compared to resuscitation with CRBCs, indicating that FRBCs and HRBCs diminish the extent of lung injury from resuscitation after TBI+HS, compared to CRBCs. See FIGS. 4A and 4C.

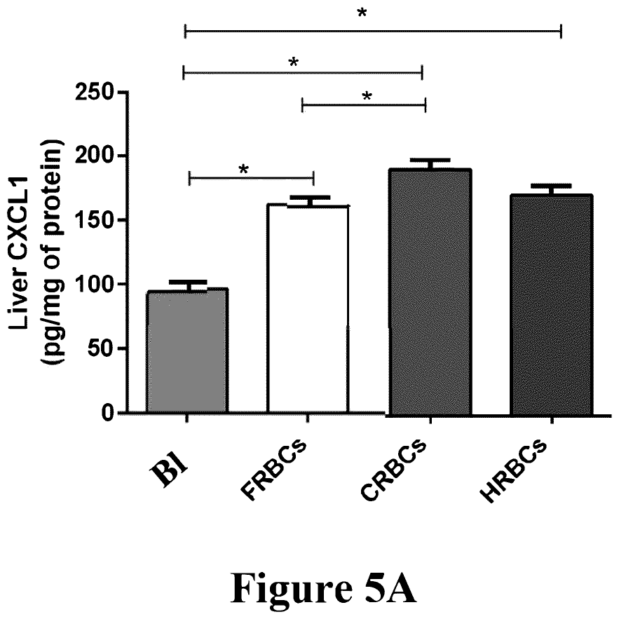

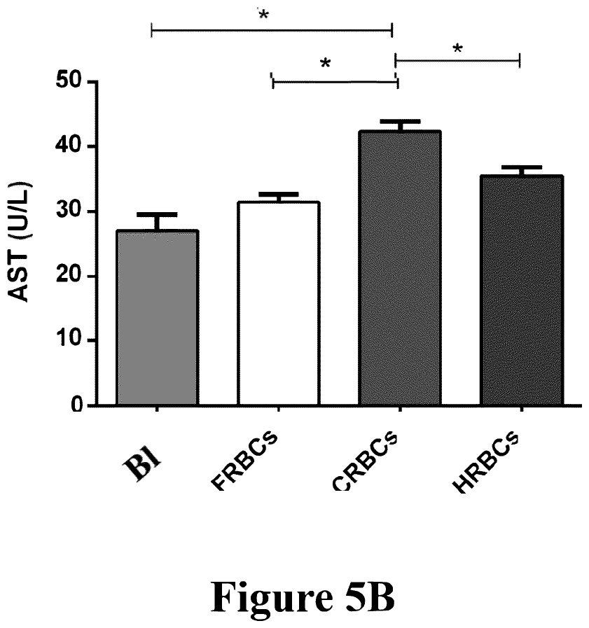

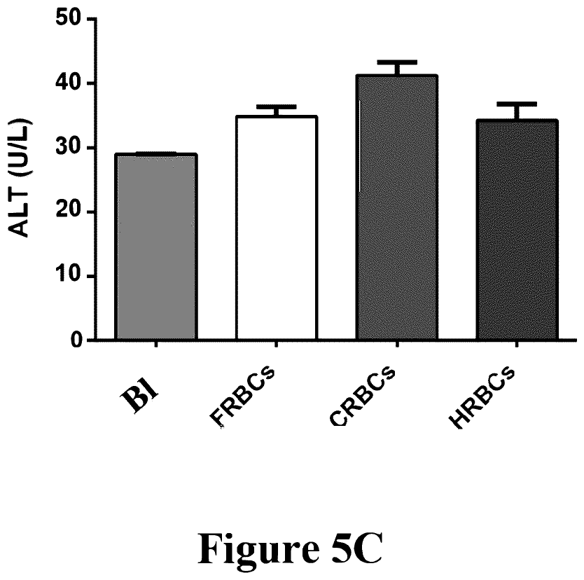

Levels of aspartate aminotransferase (AST), alanine aminotransferase (ALT), and CXCL1 in the liver are analyzed to determine liver damage. Resuscitation after TBI+HS increases levels of CXCL1, AST, and ALT in the liver compared to Bl. See FIGS. 5A-5C. Resuscitation with FRBCs and HRBCs reduces levels of CXCL1 and AST in the lung compared to resuscitation CRBCs, indicating that FRBCs and HRBCs diminish the extent of liver injury from resuscitation after TBI+HS, compared to CRBCs. The CXCL1 level is slightly higher in HRBCs compared to FRBCs. See FIGS. 5A and 5B.

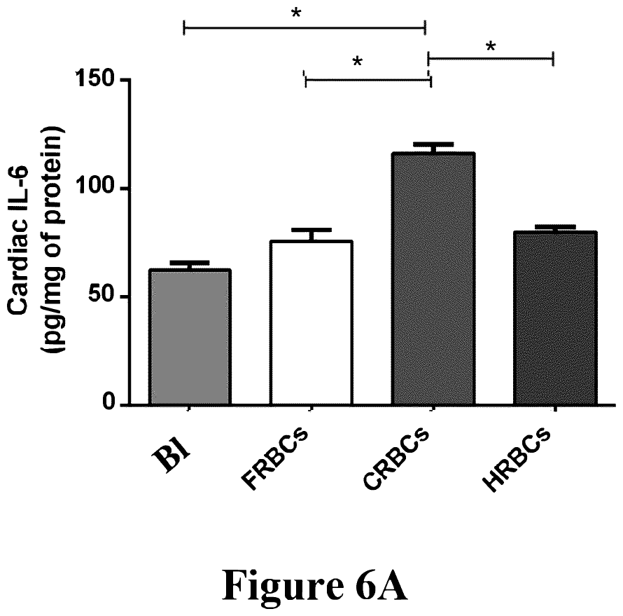

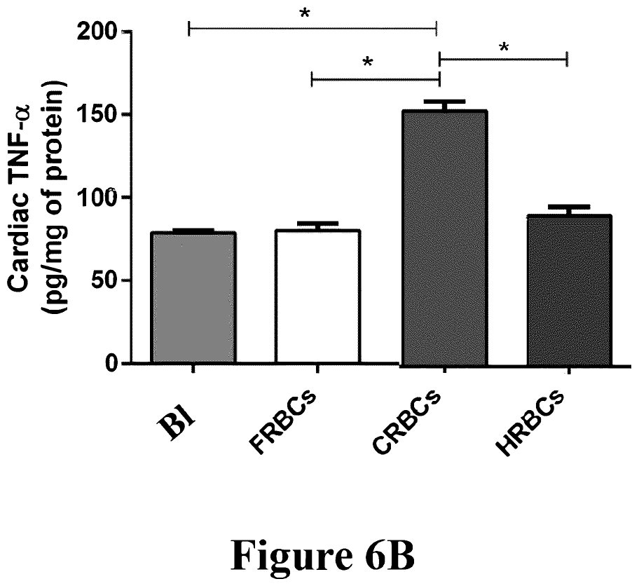

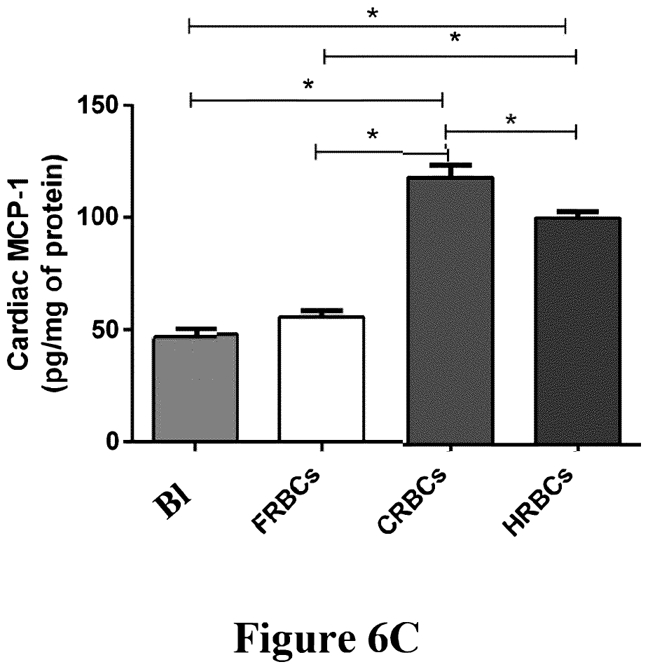

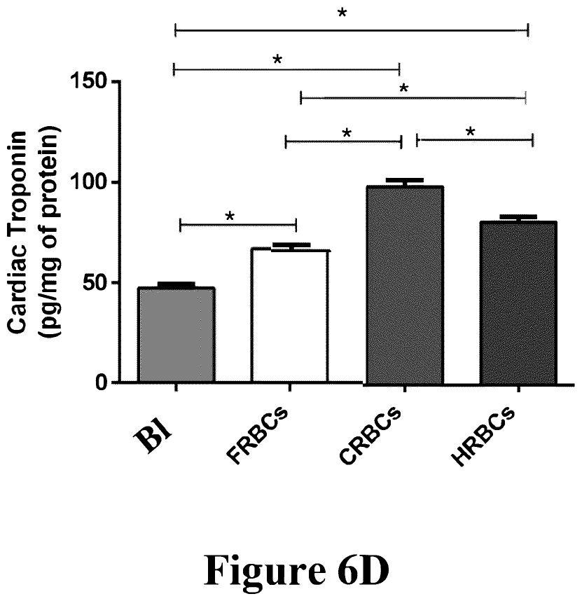

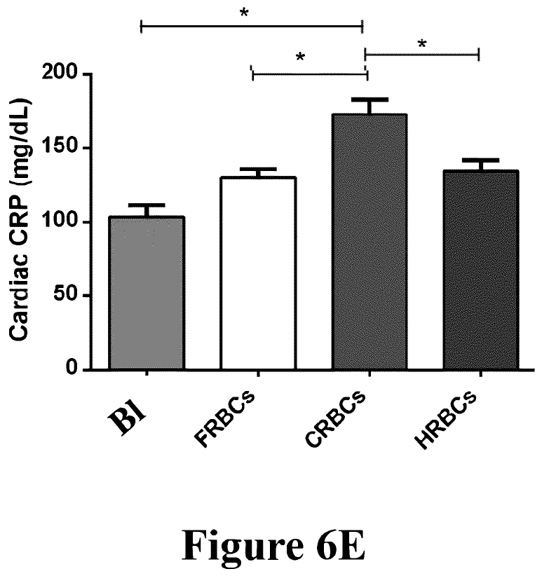

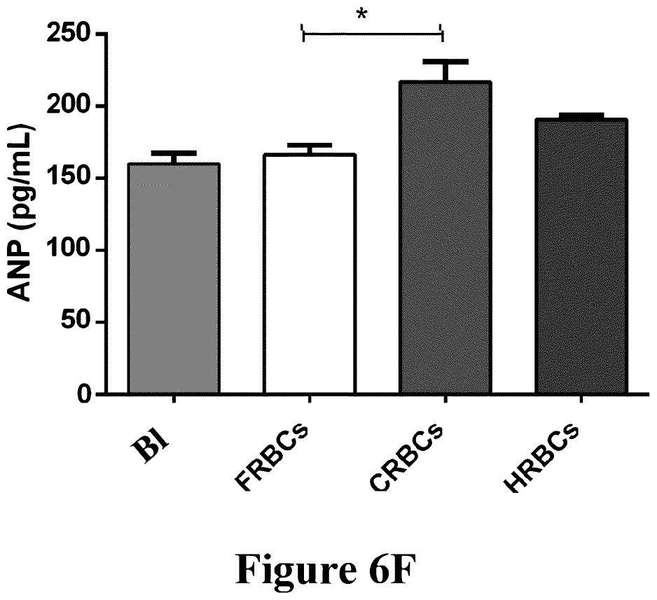

Levels of cardiac interleukin-6 (IL-6), tumor necrosis factor alpha (TNF-α), monocyte chemoattractant protein-1 (MCP-1), troponin, C reactive protein (CRP), and atrial natriuretic peptide (ANP) are analyzed to determine cardiovascular damage. Resuscitation after TBI+HS increases levels of IL-6, TNF-α, MCP-1, troponin, CRP, and ANP in the heart compared to Bl. See FIGS. 6A-6F. Resuscitation with FRBCs and HRBCs significantly reduces levels of IL-6, TNF-α, MCP-1, troponin, and CRP in the heart compared to resuscitation with CRBCs, indicating that FRBCs and HRBCs diminish the extent of cardiovascular injury from resuscitation after TBI+HS, compared to CRBCs. See FIGS. 6A-6E. The level of ANP is significantly higher in CRBCs compared to FRBCs. See FIG. 6F.

Levels of serum and urine creatinine, urinary neutrophil-associated lipocalin (u-NGAL) and blood urea nitrogen (BUN) are analyzed to determine kidney damage. No increases in levels of creatinine, u-NGAL, and BUN are observed compared to Bl, suggesting that no acute kidney damage is sustained from resuscitation after TBI+HS. See Table 3. BUN levels are higher for CRBCs and HRBCs compared to FRBCs. See Table 3.

Systemic inflammatory activation is generally expected after resuscitation. Levels of serum IL-6, serum CXCL1, and serum interleukin-10 (IL-10) are analyzed to determine the extent of systemic inflammation. Resuscitation after TBI+HS increases levels of serum IL-6 and serum CXCL1 compared to Bl. However, resuscitation with FRBCs and HRBCs reduces levels of serum IL-6 and serum CXCL1 compared to resuscitation with CRBCs, indicating that FRBCs and HRBCs diminish the extent of systemic inflammation from resuscitation after TBI+HS, compared to CRBCs. See Table 3. Additionally, serum IL-10 is increased compared to B1 only with CRBCs, but not with FRBCs or HRBCs, indicating that resuscitation with HRBCs decreases post-resuscitation systemic inflammation to a similar extent as FRBCs. See Table 3.

Overall, vital organ damage and inflammation is decreased in animals resuscitated with FRBCs and HRBCs compared to animals resuscitated with CRBCs.

TABLE 3
Kidney injury and inflammation

| | Baseline (Bl) | FRBCs | CRBCs | HRBCs |
|---|---|---|---|---|
| u-NGAL (ng/mL) | N/A | 595.9 ± 38.14 | 703.3 ± 32.88 | 718.5 ± 56.01 |
| Serum creatinine (mg/dL) | 1.30 ± 0.20 | 1.73 ± 0.11 | 1.77 ± 0.10 | 1.68 ± 0.11 |
| Urine creatinine (mg/dL) | N/A | 1.38 ± 0.12 | 1.56 ± 0.08 | 1.42 ± 0.11 |
| BUN (mg/dL) | 1.08 ± 0.06 | 0.84 ± 0.05 | 1.17 ± 0.02* | 1.07 ± 0.05* |
| Serum IL-6 (pg/dL) | 96.1 ± 0.9 | 173.1 ± 6.0‡ | 267.7 ± 8.8‡* | 206.5 ± 8.11‡*† |
| Serum CXCL1 (pg/dL) | 91.0 ± 2.4 | 180.6 ± 8.6‡ | 294.8 ± 15.8‡* | 246.2 ± 11.9‡*† |
| Serum IL-10 (pg/dL) | 96.5 ± 2.1 | 147.0 ± 5.5 | 184.5 ± 14.7‡ | 159.3 ± 7.5 |
| Norepinephrine (pg/dL) | 268.1 ± 36.3 | 349.5 ± 9.5 | 381.8 ± 19.4‡ | 330.6 ± 8.5 |
| Epinephrine (pg/dL) | 267.6 ± 39.3 | 402.6 ± 28.2 | 520.0 ± 26.2‡* | 405.7 ± 14.2† |

Data are presented as mean ± standard error (SE).
‡= p < 0.05 compared to baseline (Bl);
*= p < 0.05 compared to fresh red blood cells (FRBCs);
†= p < 0.05 compared to conventionally stored red blood cells (CRBCs).
Bl, n = 2; FRBCs, n = 8; CRBCs, n = 9; hypoxically stored red blood cells (HRBCs), n = 9.

### Example 6: Systemic Oxidative Stress after Resuscitation

A mechanism potentially associated with unwanted post-transfusion side effects is oxidative stress. Without being bound by theory, it is thought that oxidative stress occurs due to an imbalance between reactive oxygen species and antioxidant factors. Levels of enzymes and metabolites associated with the oxidative stress pathway (including antioxidants such as superoxide dismutase, catalase, and glutathione, and products of oxidative degradation such as thiobarbituric acid reactive substances (TBARS) and 8-hydroxy-2′-deoxyguanosine (8OHdG)) are analyzed to measure oxidative stress in harvested tissue homogenates and plasma.

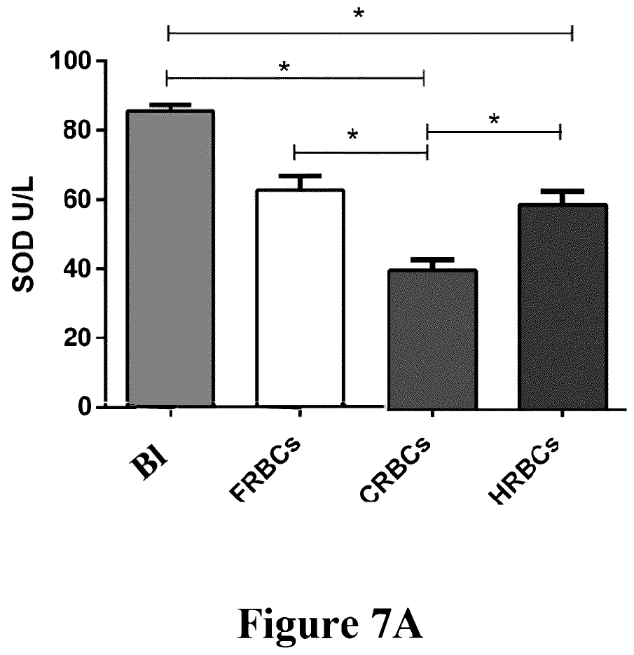

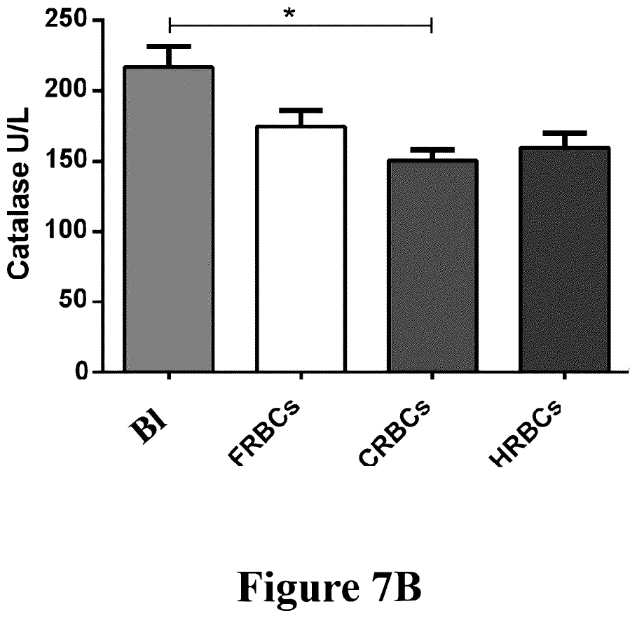

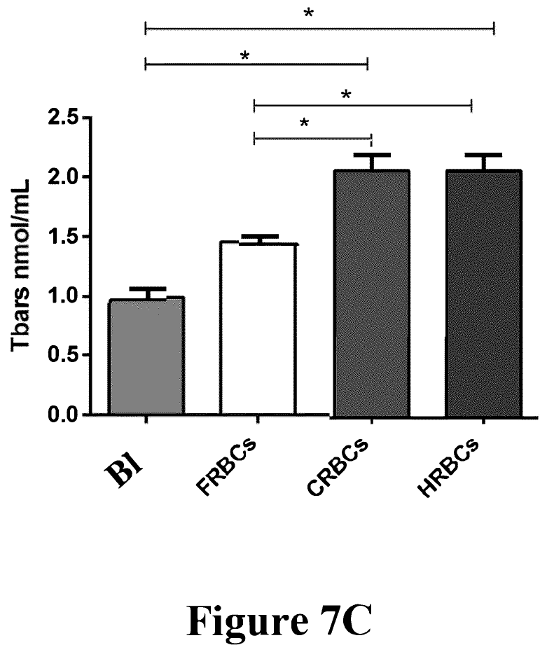

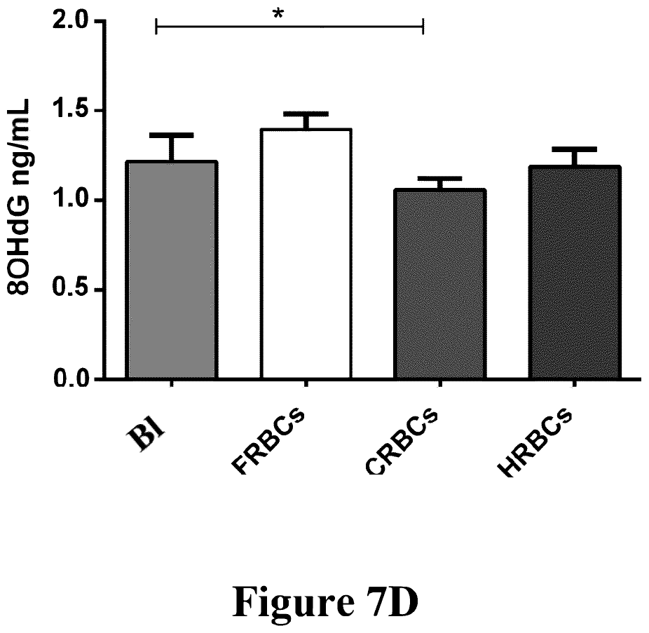

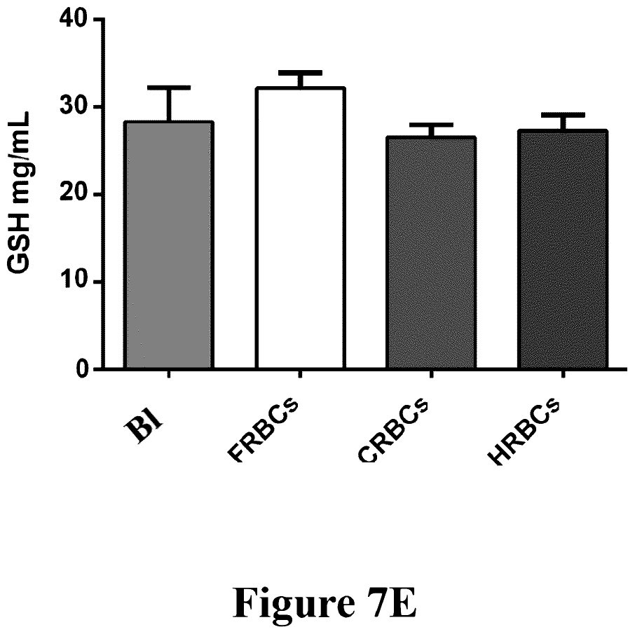

As shown in FIGS. 7A and 7B, resuscitation after TBI+HS reduces levels of superoxide dismutase (SOD) and catalase compared to Bl. In comparison to B1 and FRBCs, resuscitation with CRBCs shows a decrease in superoxide dismutase (SOD) and catalase, while TBARS (thiobarbituric acid reactive substances) shows an increase, suggesting an oxidative imbalance post-transfusion. See FIG. 7C. However, resuscitation with HRBCs ameliorates this oxidative imbalance, presenting a higher level of SOD and catalase when compared to CRBCs and no different from FRBCs. These results suggest that hypoxically stored RBCs show no difference from fresh RBCs in resuscitating from HS accompanied with TBI. Furthermore, hypoxically stored RBCs decreased organ injury and ameliorated oxidative stress compared to conventionally stored RBCs. No changes were observed in levels of GSH among groups. See FIG. 7E.

### Example 7: Iron Metabolism after Resuscitation

About 80% of the iron in the human body is associated with erythrocyte hemoglobin. Although essential for physiological health, iron can contribute to oxidative damage. Divalent ferrous iron cation (Fe²⁺) is able to react with hydrogen peroxide to generate reactive oxygen species (ROS). The resulting ROS can cause oxidative damage and lead to lipid peroxidation and tissue injury.

Ferritin is a protein that binds to excess iron cations in the body. Levels of ferritin in serum, liver, spleen, and heart are analyzed to determine iron metabolism after resuscitation. As shown in Table 4, resuscitation after TBI+HS increases levels of ferritin compared to Bl. However, resuscitation with FRBCs and HRBCs reduces levels of ferritin in serum, spleen, liver, and heart compared to resuscitation with CRBCs, indicating that FRBCs and HRBCs reduce the amount of iron cations present after resuscitation after TBI+HS, relative to CRBCs.

TABLE 4
Iron metabolism

| | Baseline (Bl) | FRBCs | CRBCs | HRBCs |
|---|---|---|---|---|
| Total bilirubin | 3.7 ± 0.4 | 3.9 ± 0.3 | 6.1 ± 0.2‡* | 4.8 ± 0.2† |
| Serum ferritin | 107.7 ± 6.5 | 183.4 ± 3.7‡ | 226.5 ± 11.01‡* | 190.7 ± 4.9‡† |
| Spleen L-ferritin | 209.2 ± 19.6 | 356.9 ± 11.3‡ | 381.5 ± 18.6‡ | 301.2 ± 8.5‡*† |
| Spleen H-ferritin | 122.0 ± 22.0 | 158.1 ± 6.1 | 167.2 ± 10.6 | 176.9 ± 8.5 |
| Liver L-ferritin | 195.3 ± 3.0 | 272.3 ± 10.2‡ | 305.5 ± 10.1‡ | 265.1 ± 12.9‡ |
| Liver H-ferritin | 200.5 ± 17.5 | 295.3 ± 4.5‡ | 303.7 ± 12.0‡ | 226.4 ± 226.4*† |
| Heart L-ferritin | 51.4 ± 5.1 | 77.7 ± 3.9‡ | 106.8 ± 3.8‡* | 91.8 ± 3.1‡*† |
| Heart H-ferritin | 84.4 ± 2.3 | 142.1 ± 5.4‡ | 150.6 ± 3.3‡ | 150.8 ± 5.2‡ |

Data are presented as mean ± standard error (SE).
‡= p < 0.05 compared to baseline (Bl);
*= p < 0.05 compared to fresh red blood cells (FRBCs);
†= p < 0.05 compared to conventionally stored red blood cells (CRBCs).
Bl, n = 2; FRBCs, n = 8; CRBCs, n = 9; hypoxically stored red blood cells (HRBCs), n = 9.

### Example 8: Hemolysis Recirculation Challenge

Hemolysis is measured for each blood storage condition (FRBCS, n=3; CRBCs, n=3; HRBCs, n=2). Briefly, 1.2 millimeters (mL) of shed blood from rats undergoing the TBI+HS procedure is mixed with 0.8 mL of stored blood used for resuscitation in a glass vial. The blood is allowed to sit at 37 °C by placing the glass vial into a water bath for a minimum of one hour. Then, the vial is connected to a P66 peristaltic pump (Harvard Apparatus, Holliston, MA) fitted with a tubing with a 0.08 centimeter (cm) diameter, as has been previously described in Jones et al., “Older Blood is Associated With Increased Mortality and Adverse Events in Massively Transfused Trauma Patients: Secondary Analysis of the PROPPR Trial,” Annals of Emergency Medicine, 73(6):650-661 (2019) (incorporated herein by reference in its entirety). The pump is run at 100 revolutions per minute (RPM) for one hour, with blood samples taken at 0, 10, 30, and 60 minutes during the run. Each sample is measured for hematocrit (Hct) using a microcapillary centrifuge, and both plasma hemoglobin (pHb) and total hemoglobin (tHb) are measured using a Hemocue Hb 201+. See FIGS. 2A-2H, particularly FIGS. 2C, 2D, and 2G.

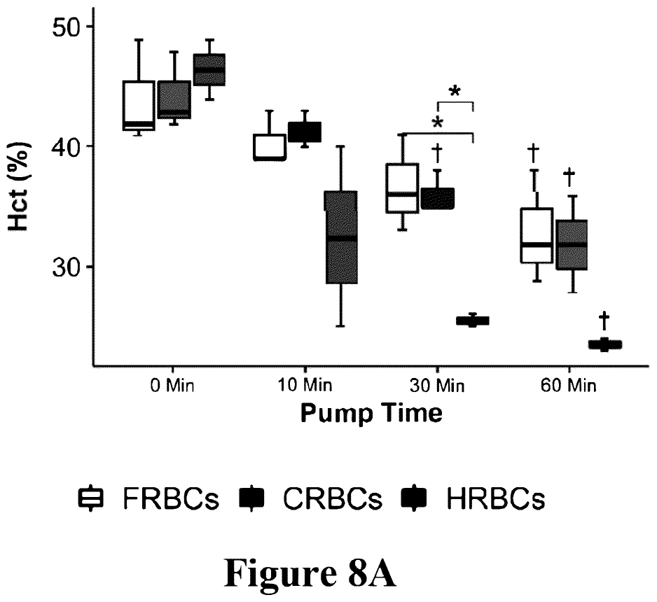

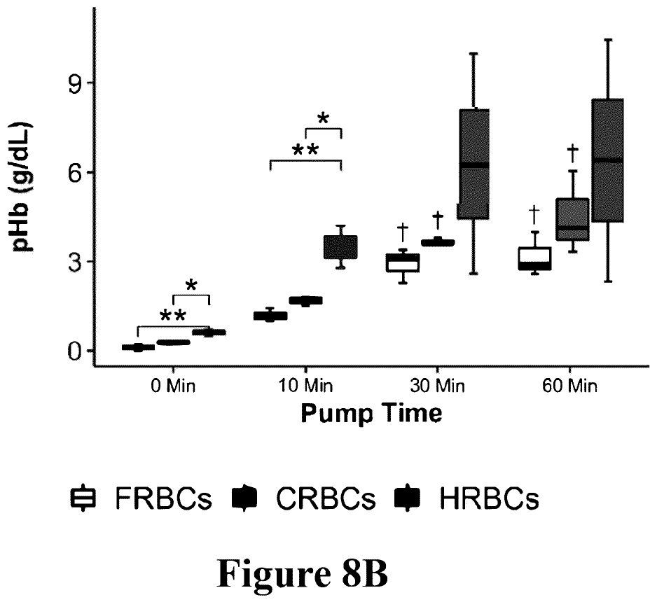

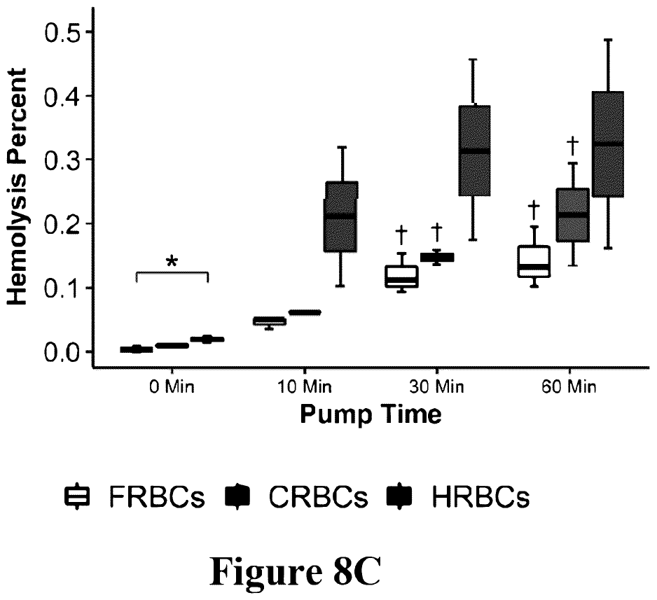

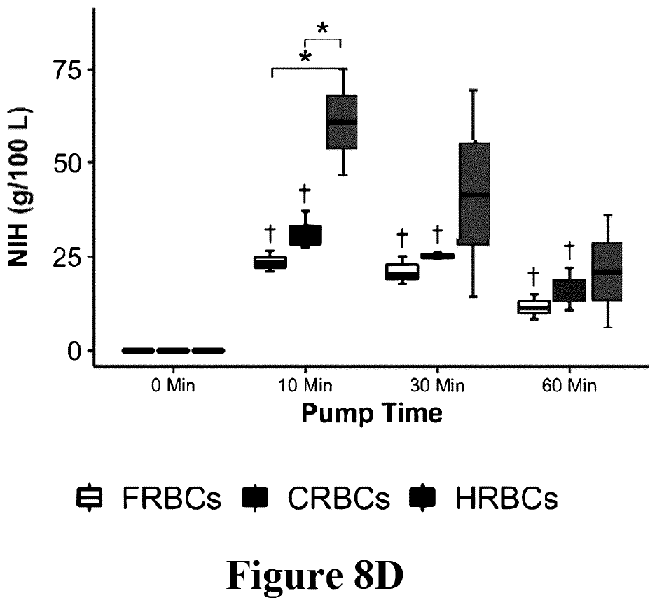

Changes in hemolysis during the recirculation challenge are shown in FIGS. 8A-8D. At 60 minutes, all three groups of blood (FRBCs, CRBCs, and HRBCs) have significantly lower Hct compared to before recirculation due to hemolysis. See FIG. 8A. Notably, ghost cells are present at 60 minutes of hemolysis, but are not quantifiable. The plasma hemoglobin (pHb) level is statistically higher in HRBCs compared to CRBCs and FRBCs before recirculation and at 10 minutes after recirculation. Without being bound by theory, since the blood used in the analysis is a combination of shed blood and the blood used for resuscitation, the early hemolysis levels in the HRBCs, especially at baseline (e.g., 0 minutes), may be attributable to the shaking of the oxygen depletion device during the deoxygenation stage before storage. However, after 10 minutes, there is no statistically significant differences in pHb levels between the three groups. At 30 minutes and 60 minutes of recirculation, CRBCs and FRBCs have significantly higher pHb levels compared to before recirculation. See FIG. 8B. There are no differences between groups of blood or hemolysis timepoints for total hemoglobin (tHb).

Hemolysis percentage and normalized index of hemolysis (NIH; measured in grams per 100 liters (g/100 L)) are calculated using the direct measures of hemoglobin (Hb) and Hct, and the following equations:

$$Hemolysis Percentage = \frac{pHb}{tHb} ( 1 - Hct )$$

$$Normalized Index of Hemolysis ( NIH ) = Δ pHb \times V \times \frac{( 100 - Hct )}{100} \times \frac{100}{Q \times t}$$

Hemolysis percentage is significantly higher in HRBCs compared to FRBCs before recirculation. At 30 minutes and 60 minutes of recirculation, the hemolysis percentages for both CRBCs and FRBCs are significantly higher compared to before recirculation. See FIG. 8C. At all timepoints past 0 minutes, both FRBCs and CRBCs have higher NIH levels compared to 0 minutes of hemolysis measurements. After 10 minutes, the HRBCs have a significantly higher NIH compared to both FRBCs and CRBCs. See FIG. 8D.

### Example 9: Statistical Analysis

All values are expressed as mean±SE. Data with multiple timepoints are analyzed using Two-Way Analysis of Variance (ANOVA) for repeated measurements. Tissues and plasma markers evaluated after reperfusion are analyzed using One-Way Analysis of Variance (ANOVA). When appropriate, post hoc analyses are performed with the Tukey multiple comparisons test. All statistics are calculated using Graph-Pad Prism 6 (GraphPad Software, Inc., San Diego, CA). Results are considered significant if p<0.05.

### Example 10: Rat Model of Resuscitation after Traumatic Brain Injury Accompanied by Mild Hemorrhagic Shock

Studies are performed in male Wistar rats (Charles River Laboratories, Wilmington, MA) weighing 350-400 g. Animal handling and care follows the National Institutes of Health (NIH) Guide for Care and Use of Laboratory Animals, and all protocols are approved by the University of California San Diego Institutional Animal Care and Use Committee. All methods are carried out in accordance with the Animal Research: Reporting of In Vivo Experiments (ARRIVE) guidelines. Behavioral and motor functions are analyzed in TBI+HS animals at baseline (also referred to as “Bl” herein), prior to induction of TBI+HS. Briefly, animals are anesthetized by administering 5%/vol isoflurane in compressed room air (Drägerwerk AG, Lübeck, Germany) to induce anesthesia, then maintained at 1.5%/vol isoflurane thereafter, except for an increase to 2.5%/vol isoflurane during surgical procedure. Animals are placed on a heating pad to preserve core body temperature at 37 °C and allowed to freely breathe from a nosecone delivering the anesthetic. Animals are instrumented with a right femoral artery catheter for hemodynamic assessment and a right femoral vein catheter for blood withdrawal and intravenous infusion. Animals are allowed to stabilize for 10 minutes. Baseline measurements are collected upon stabilization.

Before induction of traumatic brain injury (TBI), animals are transferred to a stereotaxic apparatus and placed in the ventral position. Isoflurane is increased to 2.5%/vol for 5 minutes before induction of TBI. To induce TBI, a 5 millimeter (mm) craniotomy is performed over the animal's right cerebral cortex, and the dura is impacted with a 5.0 mm flat-tipped impactor at a velocity of 5 meters (m) per second and a dwell time of 200 microseconds (ms) via a pneumatically controlled cortical impactor (CCI) (Leica Biosystems, Vista, CA). After impaction, the head opening is surgically closed and the animals are placed back on the heating pad in the dorsal position. Isoflurane is decreased to 1.5%/vol for 10 minutes before induction of hemorrhagic shock (HS).

Before induction of HS, animals are given 10 minutes to stabilize after cortical impaction. Animals are intravenously heparinized by administering 100 international units (IU) per kg of heparin to ensure patency of the catheters during the study. Animals are hemorrhaged by withdrawing approximately 30 to 35% of the animal's blood volume (BV) from the femoral vein catheter until mean arterial pressure (MAP) reaches 60 mmHg, thereby placing the animals in a hypovolemic shock condition. The hypovolemic shock condition is maintained at a MAP of 60 mmHg for 60 minutes by withdrawing additional volumes of blood when the MAP is no longer 60 mmHg. Animals neither induced with TBI-HS nor resuscitated are placed in a sham group. TBI-HS induced animals are randomly assigned to either the fresh red blood cells (FRBCs), conventionally stored red blood cells (CRBCs), or hypoxically stored red blood cells (HRBCs) group. Resuscitation (also referred to as reperfusion herein) is implemented by infusion of previously processed and stored RBCs (FRBCs, CRBCs, or HRBCs; see Example 1) at a rate of 300 microliters minute until the amount of infused blood reaches about 70% of the amount of blood withdrawn during the HS procedure. Animals are monitored for 120 minutes after resuscitation, and subsequently, animals are assessed for neurological outcome for 28 days.

Behavior assessments are performed on animals to evaluate sensorimotor function. Briefly, the Neuroscore assessment is a set of behavioral tests designed to assess sensorimotor function. It comprises four evaluations: (1) forelimb extension, (2) hindlimb extension, (3) resistance to lateral body movement, and (4) inclined plane performance. Each test yields a score ranging from 0 to 4, with 4 indicating normal sensorimotor function and 0 indicating a complete lack of sensorimotor function. An overall score falling between 26 and 28 signifies normal health, while a score of 20 to 25 suggests mild traumatic brain injury (TBI), and a score of 16 to 20 indicates moderate TBI. Scores of 15 or lower are indicative of severe TBI. See Hausser, N., et al., “Detecting behavioral deficits in rats after traumatic brain injury,” J. Vis. Exp., 30; (131):56044 (2018).

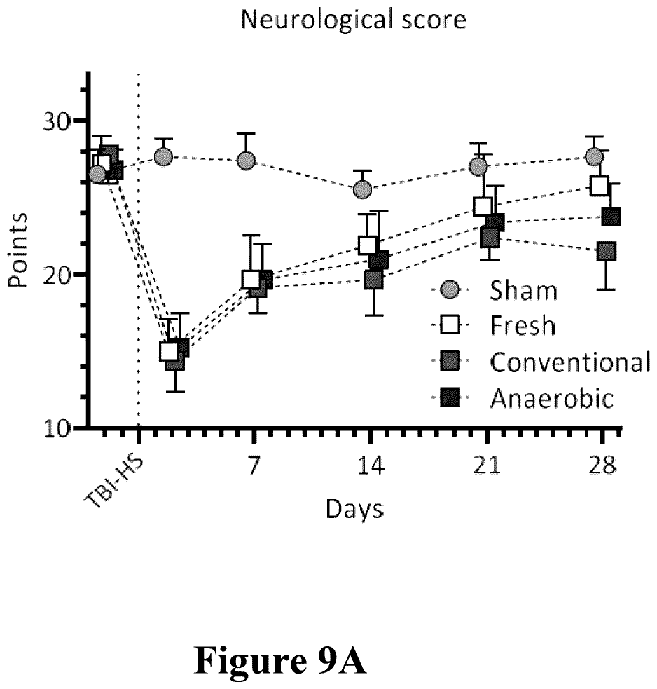

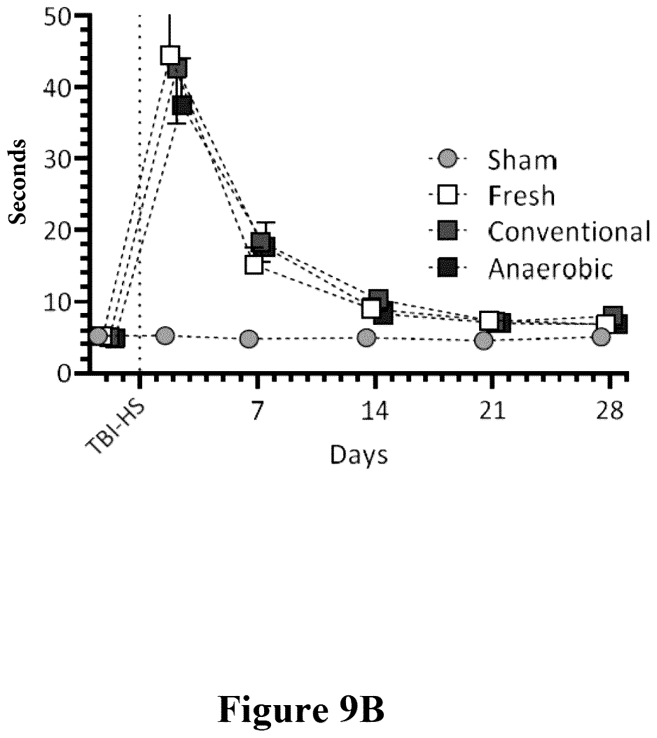

Animals are also assessed for their ability to walk through a traverse beam (FIG. 9B). Briefly, the beam-walking assessment is a test designed to evaluate motor coordination and movement integration in animals. In this behavioral evaluation, animals' ability to traverse a transverse beam is assessed. Prior to the test, the animals are acclimated to the transverse beam that is 120 centimeters long, 2.5 centimeters wide, and positioned 45 centimeters above the floor. This assessment considers traversal time—the time it takes for the animal to move from the starting platform to the safety enclosure—and a scoring system. The scoring system ranging from 6 to 0 is employed as follows:
6 points: The animal crosses the beam without any foot-slips, indicating normal function.
5 points: The animal crosses the beam with one to five foot-slips.
4 points: The animal experiences six or more foot-slips while crossing the beam.
3 points: The animal successfully traverses the beam, but a limb that is not functioning properly does not contribute to forward locomotion.
2 points: The animal falls off the beam while walking.
1 point: The animal is unable to complete the crossing but does not fall off the beam.
0 points: The rat falls off the beam without successfully traversing it.
   See Ohlsson, A. L., et al., “Environment influences functional outcome of cerebral infarction in rats,” Stroke 26, 644-649 (1995).

As shown in FIG. 9A, resuscitation with anaerobic blood restores neurological function post TBI-HS. Animals resuscitated with HRBCs (Anaerobic) have neurological scores similar to animals treated with FRBCs. FIG. 9B also displays that the time taken to walk a transverse beam for animals resuscitated with HRBCs was similar to that of animals resuscitated with FRBCs throughout the 28-day observation period.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure.

Therefore, it is intended that the present disclosure not be limited to the particular embodiments disclosed as the best mode contemplated for carrying out the present disclosure, but that the present disclosure will include all embodiments falling within the scope and spirit of the appended claims.

1. A method of treating a traumatic brain injury (TBI) accompanied by hemorrhagic shock in a patient in need thereof comprising administering oxygen reduced blood having an oxygen saturation (SO₂) of 20% or less prior to and during storage to the patient, wherein
the patient exhibits a Glasgow Coma Scale (GCS) score of 12 or less prior to the administering,
the patient has a shock index (SI) of greater than 0.9 prior to the administering, and
wherein the patient exhibits a GCS score of at least 13 after the administering, and wherein the hemorrhagic shock is reversed after the administering.

2. The method of claim 1, wherein the patient has improved function after the administering, wherein the improved function is selected from the group consisting of improved pulmonary function, improved hepatic function, improved splenic function, improved cardiac function, improved renal function, and any combination thereof.

3. The method of claim 1, wherein the patient has a reduced level of CXC motif chemokine ligand 1 (CXCL1), myeloperoxidase (MPO), lung neutrophils, alanine transaminase (ALT), aspartate transaminase (AST), alkaline phosphatase (ALP), bilirubin, interleukin 6 (IL-6), monocyte chemoattractant protein-1 (MCP1), ferritin, albumin, albumin plus globulin (total protein), tumor necrosis factor alpha (TNF alpha), troponin, plasma C reactive protein (CRP), plasma atrial natriuretic peptide (ANP), neutrophil gelatinase-associated lipocalin (NGAL), urine creatinine, serum creatinine, blood urea nitrogen (BUN), or any combination thereof after the administering.

4. The method of claim 3, wherein the bilirubin level in the patient is reduced by at least 20% after the administering, compared to a TBI patient undergoing hemorrhagic shock having been administered the same amount of non-oxygen reduced conventionally stored blood.

5. The method of claim 4, wherein the bilirubin level is reduced to between 5 millimoles per liter (mmol/L) and 120 mmol/L after the administering.

6. The method of claim 3, wherein the patient has a troponin level that is reduced by at least 5% after the administering, compared to a TBI patient undergoing hemorrhagic shock having been administered the same amount of non-oxygen reduced conventionally stored blood.

7. The method of claim 3, wherein the MCP1 level in the patient is reduced by at least 20% after the administering, compared to a TBI patient undergoing hemorrhagic shock having been administered the same amount of non-oxygen reduced conventionally stored blood.

8. The method of claim 7, wherein the MCP1 level is reduced to between 50 picograms per milliliter (pg/mL) and 200 pg/mL of plasma after the administering.

9. The method of claim 3, wherein the ferritin level in the patient is reduced by between 5% to 20% after the administering, compared to a TBI patient undergoing hemorrhagic shock having been administered the same amount of non-oxygen reduced conventionally stored blood.

10. The method of claim 1, wherein the patient has a hematocrit of less than 35% prior to the administering.

11. A method of improving a function in a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein the function is selected from the group consisting of hepatic function, splenic function, cardiac function, pulmonary function, and renal function.

12. The method of claim 11, wherein the function is hepatic function, and wherein the patient has a bilirubin level that is reduced by at least 20% after the administering compared to a TBI patient undergoing hemorrhagic shock having been administered the same amount of non-oxygen reduced conventionally stored blood.

13. The method of claim 11, wherein the function is splenic function, and wherein a monocyte chemoattractant protein-1 (MCP1) level in the patient is reduced by at least 20% after the administering compared to a TBI patient undergoing hemorrhagic shock having been administered the same amount of non-oxygen reduced conventionally stored blood.

14. The method of claim 13, wherein the MCP1 level is reduced to between 50 picograms per milliliter (pg/mL) and 200 pg/mL after the administering.

15. The method of claim 11, wherein the function is cardiac function and wherein a troponin level in the patient is reduced by at least 5% after the administering compared to a TBI patient undergoing hemorrhagic shock having been administered the same amount of non-oxygen reduced conventionally stored blood.

16. (canceled)

17. The method of claim 11, wherein the function is pulmonary function, and wherein a myeloperoxidase (MPO) level in the patient is reduced after the administering compared to a TBI patient undergoing hemorrhagic shock having been administered the same amount of non-oxygen reduced conventionally stored blood.

18. The method of claim 11, wherein the function is renal function and wherein a urine creatinine level in the patient is reduced by at least 5% after the administering compared to a TBI patient undergoing hemorrhagic shock having been administered the same amount of non-oxygen reduced conventionally stored blood.

19. The method of claim 18, wherein the urine creatinine level is reduced to between about 200 milligrams per deciliter (mg/dl) and about 350 mg/dL after the administering.

20. (canceled)

21. A method of modulating a stress level marker in the plasma of a traumatic brain injury (TBI) patient in need thereof, comprising administering oxygen reduced blood having an oxygen saturation (SO₂) of 20% or less prior to and during storage to a patient having a traumatic brain injury accompanied by hemorrhagic shock, wherein the stress level marker is selected from the group consisting of epinephrine, norepinephrine, cortisol, and any combination thereof.

22. (canceled)

23. (canceled)

24. The method of claim 12, wherein the bilirubin level is reduced to between 5 millimoles per liter (mmol/L) and 120 mmol/L after the administering.
